(19) United States
(12) Patent Application Publication
(10) Pub. No.: US 2024/0041050 A1
(43) Pub. Date: Feb. 8, 2024

(54) CELL PENETRATING PEPTIDE MEDIATED RNA TRANSDUCTION WITHIN INSECT CELLS

(71) Applicant: PIONEER HI-BRED INTERNATIONAL, INC., JOHNSTON, IA (US)

(72) Inventors: COURTNEY ANNE DAVIS-VOGEL, ANKENY, IA (US); EDWIN J. MENDEZ, WEST DES MOINES, IA (US); JAYAKUMAR PON SAMUEL, CARMEL, IN (US); KATHERINE A. THILGES, JOHNSTON, IA (US)

(73) Assignee: PIONEER HI-BRED INTERNATIONAL, INC., JOHNSTON, IA (US)

(21) Appl. No.: 18/257,165

(22) PCT Filed: Dec. 8, 2021
(86) PCT No.: PCT/US21/62321

§ 371 Date: Jun. 13, 2023

Related U.S. Application Data
(60) Provisional application No. 63/126,087, filed on Dec. 16, 2020.

(51) Int. Cl. A01N 63/60; C12N 15/113; C12N 15/87; C07K 19/00

(52) U.S. Cl. CPC A01N 63/60; C12N 15/113; C12N 15/87; C07K 19/00; C12N 2310/14; C12N 2310/3513

(57) ABSTRACT

A method of introducing a molecule of interest into an insect or an insect cell includes interacting a cell-penetrating peptide with a molecule of interest to form a complex. The structure is then placed in contact with an insect or an insect cell and allowing uptake of the complex into the insect or insect cell. Alternatively, an RNA polynucleotide (mRNA, or an RNAi-mediating molecule) can be introduced in an insect or an insect cell by interacting a peptide with the polynucleotide of interest to form a complex, allowing uptake of the RNA complex into the insect or insect cell, and expressing the polynucleotide in the insect or an insect cell.

### CROSS REFERENCE TO A RELATED APPLICATION

This application is a National Stage tiling of PCT International Application Ser. No. PCT/US21/62321, filed Dec. 8, 2021, which claims the benefit of U.S. Provisional Application Ser. No. 63/126,087, filed Dec. 16, 2020, the disclosures each of which are expressly incorporated herein by reference.

### REFERENCE TO A SEQUENCE LISTING

The official copy of the sequence listing is submitted electronically via EFS-Web as an XML formatted sequence listing with a file named “8540-US-PCT.xml” having a size of 87.1 kilobytes and is filed concurrently with the specification. The sequence listing contained in this XML formatted document is part of the specification and is herein incorporated by reference in its entirety.

### FIELD OF THE INVENTION

This disclosure is generally related to the field of molecular biology, and in particular embodiments, to the field of delivering an RNA molecule within insects and insect cells. In certain aspects, the RNA molecule is delivered by a cell-penetrating peptide (CPP). Further aspects include the development of a complex with the RNA molecule and the cell-penetrating peptide. Other aspects include the development of a complex with a messenger RNA (mRNA) and the cell-penetrating peptide. Some aspects include the development of a complex with an RNAi-mediating molecule and the cell-penetrating peptide. Some aspects include the development of a complex with a double-stranded RNA (dsRNA) molecule and the cell-penetrating peptide. Accordingly, this disclosure provides compositions and methods for the identification, detection, and utilization of delivering a molecule within insects and insect cells.

### BACKGROUND

Controlling insect populations in crop fields is economically necessary for modern agriculture practices. For example, the United States Department of Agriculture has estimated that corn rootworms (e.g., western corn rootworm) cause $1 billion in lost revenue each year. The deployment of transgenic plants for insect control provides an alternative to chemical insecticides. Chemical insecticide use is an imperfect insect control strategy. High populations of insect larvae, heavy rains, and improper application of the insecticide(s) may all result in inadequate insect control. Furthermore, the continual use of insecticide(s) may select for insecticide-resistant strains of insects, as well as raise significant environmental concerns due to toxicity to non-target species.

The transgenic expression of RNA inhibitory/interference (RNAi-mediating) molecules can be employed to regulate expression of target gene(s) by inhibiting RNA transcribed from an expressed target gene within a living organism. These non-protein coding RNAi-mediating molecules guide cleavage of target mRNA transcripts, thereby negatively regulating the expression of genes (Ambros (2001) Cell 107 (7):823-6; Bartel (2004) Cell 116 (2):281-97). Applications of the transgenic expression of RNAi-mediating molecules in plant cells to control insect pests are in development for utilization as crop protectants. RNAi allows for inhibition of a target gene within an insect by suppressing the expression of the mRNA of the target gene, and has been previously exemplified in transgenic plant applications. The usage of RNAi-based technology is promising for use in insect resistance management systems in crop fields. However, further study of RNAi-based insect control mechanisms brought the realization that once an insect developed resistance to a specific RNAi-mediating molecule, the resistance also applied to RNAi-mediating molecules targeting other genes (Khajuria C, et al. (2018) Development and characterization of the first dsRNA-resistant insect population from western corn rootworm, Diabrotica virgifera virgifera LeConte. PLoS ONE 13 (5): e0197059, and Yoon J S, et al Double stranded RNA binding protein, Staufen, is required for the initiation of RNAi in coleopteran insects. Proc Natl Acad Sci USA. 2018 Aug. 14; 115(33):8334-8339. doi: 10.1073/pnas.1809381115. Epub 2018 Jul. 30. PMID: 30061410; PMCID: PMC6099913.). There is an immediate need for improved compositions and methods to overcome the potential development of insect resistance to RNAi-based technology. Accordingly, there is a need for novel modes of action with activity against a variety of insect pests that may develop resistance to existing RNAi technology.

### SUMMARY

Disclosed herein are sequences, constructs, and methods for an RNA complex comprising a cell-penetrating peptide and an RNA molecule, wherein the cell-penetrating peptide is selected from SEQ ID NO:1 to SEQ ID NO:66, and wherein the one or more RNA molecules are selected from: an RNAi-mediating molecule; a double-stranded RNA molecule; a siRNA molecule; a micro-RNA molecule; and, a mRNA molecule. In some aspects, the RNA molecule is linked to the cell-penetrating peptide via a covalent bond. In other aspects, the RNA molecule is linked to the cell-penetrating peptide via a non-covalent bond. In further aspects, the RNA molecule is linked to the cell-penetrating peptide via an adapter or linker. In additional aspects, the cell-penetrating peptide is linked to the N-terminus of the RNA molecule. In other aspects, the cell-penetrating peptide is linked to the C-terminus of the RNA molecule. In further aspects, the cell-penetrating peptide is linked internally via a peptide backbone or a side chain to the RNA molecule. In additional aspects, the RNA molecule is linked to the cell-penetrating peptide at a molar ratio of between about 1:1 to about 1:1000. In further aspects, the cell-penetrating peptide is linked to the RNA molecule at a molar ratio of between about 1:1000 to 1:1.

The subject disclosure provides a method of introducing a molecule of interest into an insect cell, the method comprising: providing the insect cell; interacting the cell-penetrating peptide with an RNA molecule to form an RNA complex; placing the insect cell and the RNA complex in contact with each other; and allowing uptake of the RNA complex into the insect cell. In some aspects the RNA complex comprises a cell penetrating peptide of SEQ ID NO:1-SEQ ID NO:66 that is operably linked to an RNA molecule. In further aspects, the RNA molecule is an RNAi-mediating molecule; a double-stranded RNA molecule; a siRNA molecule; a micro-RNA molecule; or, a mRNA molecule. In some aspects, interacting the RNA molecule and cell-penetrating peptide, comprises fusing the RNA molecule and cell-penetrating peptide. In further aspects, the insect cell is selected from the group of Coleoptera, Diptera, Hymenoptera, Lepidoptera, Mallophaga, Homoptera, Hemiptera, Thysanoptera, Dermaptera, Isoptera, Anoplura, Siphonaptera, and Trichoptera. In other aspects, the mRNA molecule comprises a coding sequence. In additional aspects, the coding sequence is translated to a protein. In other aspects, the coding sequence encodes an agronomic trait. In some aspects, the agronomic trait is an insecticidal resistance trait. In further aspects, the agronomic trait comprises a transgenic trait. In other aspects, the contacting is performed ex vivo, in vivo, or in vitro.

The subject disclosure provides a double-stranded RNA (dsRNA) and cell-penetrating peptide (CPP) that are operably linked to form an RNA complex capable of downregulating the expression of a target mRNA of an insect pest. In some aspects the RNA complex comprises a cell penetrating peptide of SEQ ID NO:1-SEQ ID NO:66 that is operably linked to an RNA molecule. In further aspects, the RNA molecule is an RNAi-mediating molecule; a double-stranded RNA molecule; a siRNA molecule; a micro-RNA molecule; or, a mRNA molecule. In some aspects the target mRNA encodes a target gene. In further aspects the target mRNA of an insect pest is selected from the group consisting of a Caf1-180, RPA70, V-ATPase H, Rho1, V-ATPase C, Reptin, PPI-87B, RPS6, COPI gamma, COPI alpha, COPI beta, COPI delta, Brahma, ROP, Hunchback, RNA polymerase 11140, Sec23, Dre4, Gho, thread, ncm, RNA polymerase II-215, RNA polymerase I 1, RNA polymerase II 33, Kruppel, Spt5, Spt6, Snap25, SSJ1, CoatG, and Prp8. In further aspects, the RNA complex is applied to an insect pest. In other aspects the RNA complex causes post-transcriptional gene repression or inhibition of the target mRNA of the insect pest. In additional aspects, the insect pest is resistant to the uptake of a dsRNA that is not complexed to a CPP. In some aspects, the dsRNA is formed from two separate complementary RNA sequences. In other aspects, the dsRNA is formed from a single RNA sequence with internally complementary sequences.

The subject disclosure provides a pesticidal composition capable of inhibiting or downregulating the expression of target mRNA of an insect pest, wherein the pesticidal composition comprises an RNA complex. In some aspects the RNA complex comprises a cell penetrating peptide of SEQ ID NO:1-SEQ ID NO:66 that is operably linked to an RNA molecule. In further aspects, the RNA molecule is an RNAi-mediating molecule; a double-stranded RNA molecule; a siRNA molecule; a micro-RNA molecule; or, a mRNA molecule. In other aspects, the pesticidal composition is applied to a plant. The subject disclosure provides a crop plant or plant cell cultivated by: planting a seed of the crop plant; growing the crop plant from the planted seed; and treating the crop plant or plant cell with the pesticidal composition. In some aspects, the crop plant produces a commodity product. In other aspects, the commodity product is selected from the group consisting of protein concentrate, protein isolate, grain, meal, flour, oil, or fiber. In further aspects, the crop plant is selected from the group consisting of a dicotyledonous plant or a monocotyledonous plant. For example, the monocotyledonous plant is a Zea mays plant. Likewise, the dicotyledonous plant is a Glycine max plant.

The subject disclosure relates to a nucleic acid encoding the RNA complex. In some aspects the RNA complex comprises a cell penetrating peptide of SEQ ID NO:1-SEQ ID NO:66 that is operably linked to an RNA molecule. In further aspects, the RNA molecule is an RNAi-mediating molecule; a double-stranded RNA molecule; a siRNA molecule; a micro-RNA molecule; or, a mRNA molecule. In other aspects, a vector comprising the nucleic acid. In further aspects, an insect cell comprising the vector.

The subject disclosure relates to a method of inhibiting the growth of an insect, the method comprising: administering an effective amount of an RNA complex effective in inhibiting expression of the target mRNA of an insect pest. In some aspects the RNA complex comprises a cell penetrating peptide of SEQ ID NO:1-SEQ ID NO:66 that is operably linked to an RNA molecule. In further aspects, the RNA molecule is an RNAi-mediating molecule; a double-stranded RNA molecule; a siRNA molecule; a micro-RNA molecule; or, a mRNA molecule. In certain aspects, the target mRNA of an insect pest is selected from the group consisting of Caf1-180, RPA70, V-ATPase H, Rho1, V-ATPase C, Reptin, PPI-87B, RPS6, COPI gamma, COPI alpha, COPI beta, COPI delta, Brahma, ROP, Hunchback, RNA polymerase 11140, Sec23, Dre4, Gho, thread, ncm, RNA polymerase II-215, RNA polymerase I1, RNA polymerase II 33, Kruppel, Spt5, Spt6, Snap25, SSJ1 gene, CoatG gene, and Prp8.

The subject disclosure relates to a plant that exhibits an improvement in insect disease resistance, wherein the plant was topically treated with a composition that comprises the RNA complex, and the plant exhibits an improvement in insect disease resistance that results from suppressing the expression of the target mRNA of an insect pest. In some aspects the RNA complex comprises a cell penetrating peptide of SEQ ID NO:1-SEQ ID NO:66 that is operably linked to an RNA molecule. In further aspects, the RNA molecule is an RNAi-mediating molecule; a double-stranded RNA molecule; a siRNA molecule; a micro-RNA molecule; or, a mRNA molecule.

The subject disclosure relates to a transgenic plant that exhibits an improvement in insect disease resistance, wherein the transgenic plant expresses a composition that comprises the RNA complex, and the transgenic plant exhibits an improvement in insect disease resistance that results from suppressing the expression of the target mRNA of an insect pest. In some aspects the RNA complex comprises a cell penetrating peptide of SEQ ID NO:1-SEQ ID NO:66 that is operably linked to an RNA molecule. In further aspects, the RNA molecule is an RNAi-mediating molecule; a double-stranded RNA molecule; a siRNA molecule; a micro-RNA molecule; or, a mRNA molecule.

The subject disclosure relates to a method for insect resistance management, comprising expressing the RNA complex. In some aspects the RNA complex comprises a cell penetrating peptide of SEQ ID NO:1-SEQ ID NO:66 that is operably linked to an RNA molecule. In further aspects, the RNA molecule is an RNAi-mediating molecule; a double-stranded RNA molecule; a siRNA molecule; a micro-RNA molecule; or, a mRNA molecule. In certain aspects, the RNA complex is co-expressed with one or more insecticidal molecules that are toxic to insect pests in a transgenic plant. In other aspects, the RNA complex and the other insecticidal molecules exhibit different modes of action of insecticidal activity against the insect pests. In further aspects, the insecticidal activity is either insect mortality or insect growth inhibition. In additional aspects, said insect pest is from the Orders Coleoptera, Diptera, Hymenoptera, Lepidoptera, Mallophaga, Homoptera, Hemiptera, Thysanoptera, Dermaptera, Isoptera, Anoplura, Siphonaptera, or Trichoptera. In further aspects, the other insecticidal molecule is a Cry protein. In some aspects, the other insecticidal molecule is a VIP protein. In additional aspects, the transgenic plant is planted within a crop field. In further aspects, the RNA complex inhibits a target gene of an insect pest by suppressing the expression of the target mRNA of an insect pest.

The subject disclosure relates to a method of reducing likelihood of emergence of insect pests that are resistant to transgenic plants, comprising expressing the RNA complex within a plant. In some aspects the RNA complex comprises a cell penetrating peptide of SEQ ID NO:1-SEQ ID NO:66 that is operably linked to an RNA molecule. In further aspects, the RNA molecule is an RNAi-mediating molecule; a double-stranded RNA molecule; a siRNA molecule; a micro-RNA molecule; or, a mRNA molecule. In certain aspects, the RNA complex is expressed in combination with an insecticidal protein that has a different mode of action as compared to the RNA complex. In further aspects, said insect pest is from the Orders Coleoptera, Diptera, Hymenoptera, Lepidoptera, Mallophaga, Homoptera, Hemiptera, Thysanoptera, Dermaptera, Isoptera, Anoplura, Siphonaptera, or Trichoptera. In other aspects, the plant is planted within a crop field. In additional aspects, the RNA complex inhibits a target gene of an insect pest by suppressing the expression of the target mRNA of an insect pest.

The subject disclosure relates to a method for controlling an insect pest population, comprising contacting the insect pest population with an insecticidally-effective amount of the RNA complex. In some aspects the RNA complex comprises a cell penetrating peptide of SEQ ID NO:1-SEQ ID NO:66 that is operably linked to an RNA molecule. In further aspects, the RNA molecule is an RNAi-mediating molecule; a double-stranded RNA molecule; a siRNA molecule; a micro-RNA molecule; or, a mRNA molecule.

The subject disclosure relates to a method for controlling an insect pest population resistant to an RNAi-mediating molecule, comprising contacting the insect pest population with an insecticidally-effective amount of the RNA complex. In some aspects the RNA complex comprises a cell penetrating peptide of SEQ ID NO:1-SEQ ID NO:66 that is operably linked to an RNA molecule. In further aspects, the RNA molecule is an RNAi-mediating molecule; a double-stranded RNA molecule; a siRNA molecule; a micro-RNA molecule; or, a mRNA molecule.

The subject disclosure relates to a method of inhibiting growth or killing an insect pest, comprising contacting the insect pest with an insecticidally-effective amount of the RNA complex. In some aspects the RNA complex comprises a cell penetrating peptide of SEQ ID NO:1-SEQ ID NO:66 that is operably linked to an RNA molecule. In further aspects, the RNA molecule is an RNAi-mediating molecule; a double-stranded RNA molecule; a siRNA molecule; a micro-RNA molecule; or, a mRNA molecule.

The subject disclosure relates to a method of inhibiting growth or killing an insect pest resistant to an RNAi-mediating molecule, comprising contacting the insect pest with an insecticidally-effective amount of the RNA complex. In some aspects the RNA complex comprises a cell penetrating peptide of SEQ ID NO:1-SEQ ID NO:66 that is operably linked to an RNA molecule. In further aspects, the RNA molecule is an RNAi-mediating molecule; a double-stranded RNA molecule; a siRNA molecule; a micro-RNA molecule; or, a mRNA molecule.

The subject disclosure relates to a kit comprising the RNA complex. In some aspects the RNA complex comprises a cell penetrating peptide of SEQ ID NO:1-SEQ ID NO:66 that is operably linked to an RNA molecule. In further aspects, the RNA molecule is an RNAi-mediating molecule; a double-stranded RNA molecule; a siRNA molecule; a micro-RNA molecule; or, a mRNA molecule.

The foregoing and other features will become more apparent from the following embodiments as provided in the Claims and Detailed Description, which proceeds with reference to the accompanying Sequence Listing.

### SEQUENCE LISTING

The amino acid or nucleic acid sequences listed in the accompanying sequence listing are shown using standard letter abbreviations for peptide residues or nucleotide bases, as defined in 37 C.F.R. § 1.822. Only one strand of each nucleic acid sequence is shown, but the complementary strand and reverse complementary strand are understood as included by any reference to the displayed strand. As the complement and the reverse complement of a primary nucleic acid sequence are necessarily disclosed by the primary sequence, the complementary sequence and reverse complementary sequence of a nucleic acid sequence are included by any reference to the nucleic acid sequence unless it is explicitly stated to be otherwise (or it is clear to be otherwise from the context in which the sequence appears).

### BRIEF DESCRIPTION OF THE FIGURES

FIG. 1 Characteristics of unlabeled (A) and Cy3-labeled (B) dvssj1 frag 1 dsRNA. FIG. 1A The unlabeled (non-fluorescent) dsRNA preparation shows a strong band at the expected size of 210 bp when 21, 42, or 63 ng (lanes 1-3) are electrophoresed on a 6% Tris-boric acid-EDTA polyacrylamide gel and stained with GelRed® Nucleic Acid Gel Stain (Biotium, Hayward, CA). A faint lower band can be observed at the highest load, representing small contamination of non-dvssj1 nucleotides. These contaminating nucleotides are not observable when 2100 ng are analyzed via dynamic light scattering (DLS) on a Zetasizer Ultra (Malvern Panalytical, Malvern, United Kingdom). The graphs of percent signal intensity (first) or percent volume (second) versus diameter size in nanometers shows a single strong peak, indicating a highly pure stock. FIG. 1B The Cy3-labeled dsRNA preparation shows a band at ˜375 bp when 595 ng are electrophoresed on a 1.2% Tris-EDTA agarose gel and stained with Invitrogen™ SYBR Safe DNA Gel Stain (Thermo Fisher Scientific, Waltham, MA), a size ≥210 bp resulting from incorporation of Cy3-nucleotides. When 595 ng are analyzed via DLS, graphs of percent signal intensity (first) or percent volume (second) versus diameter size in nanometers shows a single strong peak, indicating a highly pure stock.

FIG. 2 Formation of CPP·dsRNA complex as assessed by gel shift assay. FIG. 2A Reactions were assembled as described in Example 3, with increasing amounts of MPG-YFP added to 2.2×10¹ nM Cy3-dsRNA (595.8 ng), from a molar ratio (CPP:dsRNA) of 1:4 through 8:1. After incubation, 20 μL reaction volumes were each mixed with 2 μL 10% molecular biology grade glycerol and electrophoresed on a 1.2% Tris-EDTA (TE) agarose gel and stained with Invitrogen™ SYBR Safe DNA Gel Stain (Thermo Fisher Scientific, Waltham, MA). Effects on mobility of the Cy3-dsRNA band are observed upon increasing molar ratios of MPG-YFP. An increase in apparent size of the RNA band from no CPP seen in lane 2 to a molar ratio of 8:1 in lane 13 indicates binding of MPG-YFP molecules to the RNA. The large relative size of MPG-YFP causes a large band shift. FIG. 2B Reactions were assembled as described in Example 3, with increasing amounts of CyLoP added to 770 nM dsRNA (2100 ng), from a molar ratio of 1:4 through 8:1. After incubation, 20 μL reaction volumes were each mixed with 2 μL 10% molecular biology grade glycerol and electrophoresed on a 1.2% TE agarose gel and stained with SYBR Safe DNA Gel Stain. Effects on mobility of the dsRNA band are observed upon increasing molar ratios of CyLoP. An increase in apparent size of the RNA band from no CPP seen in lane 2 to a molar ratio of 8:1 in lane 13 indicates binding of CyLoP molecules to the RNA. The smaller size of CyLoP versus MPG-YFP causes a smaller band shift. FIG. 2C Results of MPG-YFP binding to Cy3-dsRNA (described in 2A) assessed by DLS. After incubation, 20 μL reaction volumes were loaded into a capillary cuvette and capillary cell. Readings were collected using default settings for protein molecules, at 25° C. with a 15 second equilibration stage, using a Zetasizer Ultra and accompanying software (Malvern Panalytical, Malvern, United Kingdom). The dispersant was adjusted to account for the specific viscosity and refractive index of the complex formation solution prior to calculation of detected sizes. A jump in size can be seen between the MPG-YFP stock solution and first addition of Cy3-dsRNA. An increase in estimated diameter size (nm) of the single complex peak can be seen as the molar ratio of MPG-YFP in the samples increases, measured by mean intensity.

### DETAILED DESCRIPTION

Throughout the application, a number of terms are used. In order to provide a clear and consistent understanding of the specification and claims, including the scope to be given such terms, the following definitions are provided.

As used herein, the articles, “a,” “an,” and “the” include plural references unless the context clearly and unambiguously dictates otherwise.

The term “isolated”, as used herein means having been removed from its natural environment, or removed from other compounds present when the compound is first formed. The term “isolated” embraces materials isolated from natural sources as well as materials (e.g., nucleic acids and proteins) recovered after preparation by recombinant expression in a host cell, or chemically-synthesized compounds such as nucleic acid molecules, proteins, and peptides.

The term “purified”, as used herein relates to the isolation of a molecule or compound in a form that is substantially free of contaminants normally associated with the molecule or compound in a native or natural environment, or substantially enriched in concentration relative to other compounds present when the compound is first formed, and means having been increased in purity as a result of being separated from other components of the original composition. The term “purified nucleic acid” is used herein to describe a nucleic acid sequence which has been separated, produced apart from, or purified away from other biological compounds including, but not limited to polypeptides, lipids and carbohydrates, while effecting a chemical or functional change in the component (e.g., a nucleic acid may be purified from a chromosome by removing protein contaminants and breaking chemical bonds connecting the nucleic acid to the remaining DNA in the chromosome).

The term “synthetic”, as used herein refers to a polynucleotide (i.e., a DNA or RNA) molecule that was created via chemical synthesis as an in vitro process. For example, a synthetic DNA may be created during a reaction within an Eppendorf™ tube, such that the synthetic DNA is enzymatically produced from a native strand of DNA or RNA. Other laboratory methods may be utilized to synthesize a polynucleotide sequence. Oligonucleotides may be chemically synthesized on an oligo synthesizer via solid-phase synthesis using phosphoramidites. The synthesized oligonucleotides may be annealed to one another as a complex, thereby producing a “synthetic” polynucleotide. Other methods for chemically synthesizing a polynucleotide are known in the art, and can be readily implemented for use in the present disclosure.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

For the purposes of the present disclosure, a “gene,” includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, introns and locus control regions.

As used herein the terms “native” or “natural” define a condition found in nature. A “native DNA sequence” is a DNA sequence present in nature that was produced by natural means or traditional breeding techniques but not generated by genetic engineering (e.g., using molecular biology/transformation techniques).

As used herein a “transgene” is defined to be a nucleic acid sequence that encodes a gene product, including for example, but not limited to, an mRNA. In one embodiment the transgene/heterologous coding sequence is an exogenous nucleic acid, where the transgene/heterologous coding sequence has been introduced into a host cell by genetic engineering (or the progeny thereof) where the transgene/heterologous coding sequence is not normally found. In one example, a transgene/heterologous coding sequence encodes an industrially or pharmaceutically useful compound, or a gene encoding a desirable agricultural trait (e.g., an herbicide-resistance gene). In yet another example, a transgene/heterologous coding sequence is an antisense nucleic acid sequence, wherein expression of the antisense nucleic acid sequence inhibits expression of a target nucleic acid sequence. In one embodiment the transgene/heterologous coding sequence is an endogenous nucleic acid, wherein additional genomic copies of the endogenous nucleic acid are desired, or a nucleic acid that is in the antisense orientation with respect to the sequence of a target nucleic acid in a host organism.

A “gene product” as defined herein is any product produced by the gene. For example the gene product can be the direct transcriptional product of a gene (e.g., mRNA, tRNA, rRNA, antisense RNA, interfering RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation. Gene expression can be influenced by external signals, for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

As used herein the term “gene expression” relates to the process by which the coded information of a nucleic acid transcriptional unit (including, e.g., genomic DNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

As used herein, the term “nucleic acid molecule” (or “nucleic acid” or “polynucleotide”) may refer to a polymeric form of nucleotides, which may include both sense and anti-sense strands of RNA, complementary DNA (cDNA), genomic DNA, and synthetic forms and mixed polymers of the above. A nucleotide may refer to a ribonucleotide (RNA), deoxyribonucleotide (DNA), or a modified form of either type of nucleotide. A “nucleic acid molecule” as used herein is synonymous with “nucleic acid” and “polynucleotide”. A nucleic acid molecule is usually at least 10 bases in length, unless otherwise specified. The term may refer to a molecule of RNA or DNA of indeterminate length. The term includes single- and double-stranded forms of DNA and RNA. A nucleic acid molecule may include either or both naturally-occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidites, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

Transcription proceeds in a 5′ to 3′ manner along a DNA strand. This means that RNA is made by the sequential addition of ribonucleotide-5′-triphosphates to the 3′ terminus of the growing chain (with a requisite elimination of the pyrophosphate). In either a linear or circular nucleic acid molecule, discrete elements (e.g., particular nucleotide sequences) may be referred to as being “upstream” or “5′” relative to a further element if they are bonded or would be bonded to the same nucleic acid in the 5′ direction from that element. Similarly, discrete elements may be “downstream” or “3′” relative to a further element if they are or would be bonded to the same nucleic acid in the 3′ direction from that element.

A base “position”, as used herein, refers to the location of a given base or nucleotide residue within a designated nucleic acid. The designated nucleic acid may be defined by alignment (see below) with a reference nucleic acid.

Hybridization relates to the binding of two polynucleotide strands via hydrogen bonds. Oligonucleotides and their analogs hybridize by hydrogen bonding, which includes Watson-Crick, Hoogsteen or reversed Hoogsteen hydrogen bonding, between complementary bases. Generally, nucleic acid molecules consist of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide and the DNA or RNA target. The oligonucleotide need not be 100% complementary to its target sequence to be specifically hybridizable. An oligonucleotide is specifically hybridizable when binding of the oligonucleotide to the target DNA or RNA molecule interferes with the normal function of the target DNA or RNA, and there is sufficient degree of complementarity to avoid non-specific binding of the oligonucleotide to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the chosen hybridization method and the composition and length of the hybridizing nucleic acid sequences. Generally, the temperature of hybridization and the ionic strength (especially the Na⁺ and/or Mg²⁺ concentration) of the hybridization buffer will contribute to the stringency of hybridization, though wash times also influence stringency. Calculations regarding hybridization conditions required for attaining particular degrees of stringency are discussed in Sambrook et al. (ed.), Molecular Cloning: A Laboratory Manual, 2nd ed., vol. 1-3, Cold Spring Harbor Laboratory Press, Cold Spring Harbor, New York, 1989, chs. 9 and 11.

As used herein, “stringent conditions” encompass conditions under which hybridization will only occur if there is less than 50% mismatch between the hybridization molecule and the DNA target. “Stringent conditions” include further particular levels of stringency. Thus, as used herein, “moderate stringency” conditions are those under which molecules with more than 50% sequence mismatch will not hybridize; conditions of “high stringency” are those under which sequences with more than 20% mismatch will not hybridize; and conditions of “very high stringency” are those under which sequences with more than 10% mismatch will not hybridize.

In particular embodiments, stringent conditions can include hybridization at 65° C., followed by washes at 65° C. with 0.1×SSC/0.1% SDS for 40 minutes. The following are representative, non-limiting hybridization conditions: Very High Stringency: Hybridization in 5×SSC buffer at 65° C. for 16 hours; wash twice in 2×SSC buffer at room temperature for 15 minutes each; and wash twice in 0.5×SSC buffer at 65° C. for 20 minutes each. High Stringency: Hybridization in 5×-6×SSC buffer at 65-70° C. for 16-20 hours; wash twice in 2×SSC buffer at room temperature for 5-20 minutes each; and wash twice in 1×SSC buffer at 55-70° C. for 30 minutes each. Moderate Stringency: Hybridization in 6×SSC buffer at room temperature to 55° C. for 16-20 hours; wash at least twice in 2×-3×SSC buffer at room temperature to 55° C. for 20-30 minutes each.

In particular embodiments, specifically hybridizable nucleic acid molecules can remain bound under very high stringency hybridization conditions. In these and further embodiments, specifically hybridizable nucleic acid molecules can remain bound under high stringency hybridization conditions. In these and further embodiments, specifically hybridizable nucleic acid molecules can remain bound under moderate stringency hybridization conditions.

As used herein, the term “oligonucleotide” refers to a short nucleic acid polymer. Oligonucleotides may be formed by cleavage of longer nucleic acid segments, or by polymerizing individual nucleotide precursors. Automated synthesizers allow the synthesis of oligonucleotides up to several hundred base pairs in length. Because oligonucleotides may bind to a complementary nucleotide sequence, they may be used as probes for detecting DNA or RNA. Oligonucleotides composed of DNA (oligodeoxyribonucleotides) may be used in PCR, a technique for the amplification of DNA sequences. In PCR, the oligonucleotide is typically referred to as a “primer”, which allows a DNA polymerase to extend the oligonucleotide and replicate the complementary strand.

The terms “percent sequence identity” or “percent identity” or “identity” are used interchangeably to refer to a sequence comparison based on identical matches between correspondingly identical positions in the sequences being compared between two or more amino acid or nucleotide sequences. The percent identity refers to the extent to which two optimally aligned polynucleotide or peptide sequences are invariant throughout a window of alignment of components, e.g., nucleotides or amino acids. Hybridization experiments and mathematical algorithms known in the art may be used to determine percent identity. Many mathematical algorithms exist as sequence alignment computer programs known in the art that calculate percent identity. These programs may be categorized as either global sequence alignment programs or local sequence alignment programs.

Global sequence alignment programs calculate the percent identity of two sequences by comparing alignments end-to-end in order to find exact matches, dividing the number of exact matches by the length of the shorter sequences, and then multiplying by 100. Basically, the percentage of identical nucleotides in a linear polynucleotide sequence of a reference (“query”) polynucleotide molecule as compared to a test (“subject”) polynucleotide molecule when the two sequences are optimally aligned (with appropriate nucleotide insertions, deletions, or gaps).

Local sequence alignment programs are similar in their calculation, but only compare aligned fragments of the sequences rather than utilizing an end-to-end analysis. Local sequence alignment programs such as Basic Local Alignment Search Tool (BLAST) can be used to compare specific regions of two sequences. A BLAST comparison of two sequences results in an E-value, or expectation value, that represents the number of different alignments with scores equivalent to or better than the raw alignment score, S, that are expected to occur in a database search by chance. The lower the E-value, the more significant the match. Because database size is an element in E-value calculations, E-values obtained by BLASTing against public databases, such as GENBANK, have generally increased over time for any given query/entry match. In setting criteria for confidence of polypeptide function prediction, a “high” BLAST match is considered herein as having an E-value for the top BLAST hit of less than 1e⁻³⁰; a medium BLAST E-value is 1e⁻³⁰ to 1e⁻⁸; and a low BLAST E-value is greater than 1e⁻⁸. The protein function assignment in the present disclosure is determined using combinations of E-values, percent identity, query coverage and hit coverage. Query coverage refers to the percent of the query sequence that is represented in the BLAST alignment. Hit coverage refers to the percent of the database entry that is represented in the BLAST alignment. In one embodiment of the disclosure, function of a query polypeptide is inferred from function of a conserved protein sequence where either (1) hit_p<1e⁻³⁰ or % identity >35% AND query_coverage>50% AND hit_coverage>50%, or (2) hit_p<1e⁻⁸ AND query_coverage>70% AND hit_coverage>70%. Methods for aligning sequences for comparison are well-known in the art. Various programs and alignment algorithms are described. In an embodiment, the subject disclosure relates to calculating percent identity between two polynucleotides or amino acid sequences using an AlignX alignment program of the Vector NTI suite (Invitrogen, Carlsbad, CA). The AlignX alignment program is a global sequence alignment program for polynucleotides or proteins. In an embodiment, the subject disclosure relates to calculating percent identity between two polynucleotides or amino acid sequences using the MegAlign program of the LASERGENE bioinformatics computing suite (MegAlign™ (©1993-2016). DNASTAR. Madison, WI). The MegAlign program is a global sequence alignment program for polynucleotides or proteins. In an embodiment, the subject disclosure relates to calculating percent identity between two polynucleotides or amino acid sequences using the Clustal suite of alignment programs, including, but not limited to, ClustalW and ClustalV (Higgins and Sharp (1988) Gene. December 15; 73(1):237-44; Higgins and Sharp (1989) CABIOS 5:151-3; Higgins et al. (1992) Comput. Appl. Biosci. 8:189-91). In an embodiment, the subject disclosure relates to calculating percent identity between two polynucleotides or amino acid sequences using the GCG suite of programs (Wisconsin Package Version 9.0, Genetics Computer Group (GCG), Madison, WI). In an embodiment, the subject disclosure relates to calculating percent identity between two polynucleotides or amino acid sequences using the BLAST suite of alignment programs, for example, but not limited to, BLASTP, BLASTN, BLASTX, etc. (Altschul et al. (1990) J. Mol. Biol. 215:403-10). Further examples of such BLAST alignment programs include Gapped-BLAST or PSI-BLAST (Altschul et al., 1997). In an embodiment, the subject disclosure relates to calculating percent identity between two polynucleotides or amino acid sequences using the FASTA suite of alignment programs, including, but not limited to, FASTA, TFASTX, TFASTY, SSEARCH, LALIGN etc. (Pearson (1994) Comput. Methods Genome Res. [Proc. Int. Symp.], Meeting Date 1992 (Suhai and Sandor, Eds.), Plenum: New York, NY, pp. 111-20). In an embodiment, the subject disclosure relates to calculating percent identity between two polynucleotides or amino acid sequences using the T-Coffee alignment program (Notredame, et. al. (2000) J. Mol. Biol. 302, 205-17). In an embodiment, the subject disclosure relates to calculating percent identity between two polynucleotides or amino acid sequences using the DIALIGN suite of alignment programs, including, but not limited to DIALIGN, CHAOS, DIALIGN-TX, DIALIGN-T etc. (Al Ait, et. al. (2013) DIALIGN at GOBICS Nuc. Acids Research 41, W3-W7). In an embodiment, the subject disclosure relates to calculating percent identity between two polynucleotides or amino acid sequences using the MUSCLE suite of alignment programs (Edgar (2004) Nucleic Acids Res. 32(5): 1792-1797). In an embodiment, the subject disclosure relates to calculating percent identity between two polynucleotides or amino acid sequences using the MAFFT alignment program (Katoh, et. al. (2002) Nucleic Acids Research 30(14): 3059-3066). In an embodiment, the subject disclosure relates to calculating percent identity between two polynucleotides or amino acid sequences using the Genoogle program (Albrecht, Felipe. arXiv150702987v1 [cs.DC] 10 Jul. 2015). In an embodiment, the subject disclosure relates to calculating percent identity between two polynucleotides or amino acid sequences using the HMMER suite of programs (Eddy. (1998) Bioinformatics, 14:755-63). In an embodiment, the subject disclosure relates to calculating percent identity between two polynucleotides or amino acid sequences using the PLAST suite of alignment programs, including, but not limited to, TPLASTN, PLASTP, KLAST, and PLASTX (Nguyen & Lavenier. (2009) BMC Bioinformatics, 10:329). In an embodiment, the subject disclosure relates to calculating percent identity between two polynucleotides or amino acid sequences using the USEARCH alignment program (Edgar (2010) Bioinformatics 26(19), 2460-61). In an embodiment, the subject disclosure relates to calculating percent identity between two polynucleotides or amino acid sequences using the SAM suite of alignment programs (Hughey & Krogh (January 1995) Technical Report UCSC0CRL-95-7, University of California, Santa Cruz). In an embodiment, the subject disclosure relates to calculating percent identity between two polynucleotides or amino acid sequences using the IDF Searcher (O'Kane, K. C., The Effect of Inverse Document Frequency Weights on Indexed Sequence Retrieval, Online Journal of Bioinformatics, Volume 6 (2) 162-173, 2005). In an embodiment, the subject disclosure relates to calculating percent identity between two polynucleotides or amino acid sequences using the Parasail alignment program. (Daily, Jeff. Parasail: SIMD C library for global, semi-global, and local pairwise sequence alignments. BMC Bioinformatics. 17:18. Feb. 10, 2016). In an embodiment, the subject disclosure relates to calculating percent identity between two polynucleotides or amino acid sequences using the ScalaBLAST alignment program (Oehmen C, Nieplocha J. “ScalaBLAST: A scalable implementation of BLAST for high-performance data-intensive bioinformatics analysis.” IEEE Transactions on Parallel & Distributed Systems 17 (8): 740-749 August 2006). In an embodiment, the subject disclosure relates to calculating percent identity between two polynucleotides or amino acid sequences using the SWIPE alignment program (Rognes, T. Faster Smilth-Waterman database searches with inter-sequence SIMD parallelization. BMC Bioinformatics. 12, 221 (2011)). In an embodiment, the subject disclosure relates to calculating percent identity between two polynucleotides or amino acid sequences using the ACANA alignment program (Weichun Huang, David M. Umbach, and Leping Li, Accurate anchoring alignment of divergent sequences. Bioinformatics 22:29-34, Jan. 1, 2006). In an embodiment, the subject disclosure relates to calculating percent identity between two polynucleotides or amino acid sequences using the DOTLET alignment program (Junier, T. & Pagni, M. DOTLET: diagonal plots in a web browser. Bioinformatics 16(2): 178-9 Feb. 2000). In an embodiment, the subject disclosure relates to calculating percent identity between two polynucleotides or amino acid sequences using the G-PAS alignment program (Frohmberg, W., et al. G-PAS 2.0—an improved version of protein alignment tool with an efficient backtracking routine on multiple GPUs. Bulletin of the Polish Academy of Sciences Technical Sciences, Vol. 60, 491 November 2012). In an embodiment, the subject disclosure relates to calculating percent identity between two polynucleotides or amino acid sequences using the GapMis alignment program (Flouri, T. et. al., Gap Mis: A tool for pairwise sequence alignment with a single gap. Recent Pat DNA Gene Seq. 7(2): 84-95 August 2013). In an embodiment, the subject disclosure relates to calculating percent identity between two polynucleotides or amino acid sequences using the EMBOSS suite of alignment programs, including, but not limited to: Matcher, Needle, Stretcher, Water, Wordmatch, etc. (Rice, P., Longden, I. & Bleasby, A. EMBOSS: The European Molecular Biology Open Software Suite. Trends in Genetics 16(6) 276-77 (2000)). In an embodiment, the subject disclosure relates to calculating percent identity between two polynucleotides or amino acid sequences using the Ngila alignment program (Cartwright, R. Ngila: global pairwise alignments with logarithmic and affine gap costs. Bioinformatics. 23(11): 1427-28. Jun. 1, 2007). In an embodiment, the subject disclosure relates to calculating percent identity between two polynucleotides or amino acid sequences using the probA, also known as propA, alignment program (Mückstein, U., Hofacker, IL, & Stadler, P F. Stochastic pairwise alignments. Bioinformatics 18 Suppl. 2:S153-60. 2002). In an embodiment, the subject disclosure relates to calculating percent identity between two polynucleotides or amino acid sequences using the SEQALN suite of alignment programs (Hardy, P. & Waterman, M. The Sequence Alignment Software Library at USC. 1997). In an embodiment, the subject disclosure relates to calculating percent identity between two polynucleotides or amino acid sequences using the SIM suite of alignment programs, including, but not limited to, GAP, NAP, LAP, etc. (Huang, X & Miller, W. A Time-Efficient, Linear-Space Local Similarity Algorithm. Advances in Applied Mathematics, vol. 12 (1991) 337-57). In an embodiment, the subject disclosure relates to calculating percent identity between two polynucleotides or amino acid sequences using the UGENE alignment program (Okonechnikov, K., Golosova, O. & Fursov, M. Unipro UGENE: a unified bioinformatics toolkit. Bioinformatics. 2012 28:1166-67). In an embodiment, the subject disclosure relates to calculating percent identity between two polynucleotides or amino acid sequences using the BAli-Phy alignment program (Suchard, M A & Redelings, B D. BAli-Phy: simultaneous Bayesian inference of alignment and phylogeny. Bioinformatics. 22:2047-48. 2006). In an embodiment, the subject disclosure relates to calculating percent identity between two polynucleotides or amino acid sequences using the Base-By-Base alignment program (Brodie, R., et. al. Base-By-Base: Single nucleotide-level analysis of whole viral genome alignments, BMC Bioinformatics, 5, 96, 2004). In an embodiment, the subject disclosure relates to calculating percent identity between two polynucleotides or amino acid sequences using the DECIPHER alignment program (E S Wright (2015) “DECIPHER: harnessing local sequence context to improve protein multiple sequence alignment.” BMC Bioinformatics, doi:10.1186/s12859-015-0749-z.). In an embodiment, the subject disclosure relates to calculating percent identity between two polynucleotides or amino acid sequences using the FSA alignment program (Bradley, R K, et. al. (2009) Fast Statistical Alignment. PLoS Computational Biology. 5:e1000392). In an embodiment, the subject disclosure relates to calculating percent identity between two polynucleotides or amino acid sequences using the Geneious alignment program (Kearse, M., et. al. (2012). Geneious Basic: an integrated and extendable desktop software platform for the organization and analysis of sequence data. Bioinformatics, 28(12), 1647-49). In an embodiment, the subject disclosure relates to calculating percent identity between two polynucleotides or amino acid sequences using the Kalign alignment program (Lassmann, T. & Sonnhammer, E. Kalign—an accurate and fast multiple sequence alignment algorithm. BMC Bioinformatics 2005 6:298). In an embodiment, the subject disclosure relates to calculating percent identity between two polynucleotides or amino acid sequences using the MAVID alignment program (Bray, N. & Pachter, L. MAVID: Constrained Ancestral Alignment of Multiple Sequences. Genome Res. 2004 April; 14(4): 693-99). In an embodiment, the subject disclosure relates to calculating percent identity between two polynucleotides or amino acid sequences using the MSA alignment program (Lipman, D J, et. al. A tool for multiple sequence alignment. Proc. Nat'l Acad. Sci. USA. 1989; 86:4412-15). In an embodiment, the subject disclosure relates to calculating percent identity between two polynucleotides or amino acid sequences using the MultAlin alignment program (Corpet, F., Multiple sequence alignment with hierarchical clustering. Nucl. Acids Res., 1988, 16(22), 10881-90). In an embodiment, the subject disclosure relates to calculating percent identity between two polynucleotides or amino acid sequences using the LAGAN or MLAGAN alignment programs (Brudno, et. al. LAGAN and Multi-LAGAN: efficient tools for large-scale multiple alignment of genomic DNA. Genome Research 2003 April; 13(4): 721-31). In an embodiment, the subject disclosure relates to calculating percent identity between two polynucleotides or amino acid sequences using the Opal alignment program (Wheeler, T. J., & Kececiouglu, J. D. Multiple alignment by aligning alignments. Proceedings of the 15^{th} ISCB conference on Intelligent Systems for Molecular Biology. Bioinformatics. 23, i559-68, 2007). In an embodiment, the subject disclosure relates to calculating percent identity between two polynucleotides or amino acid sequences using the PicXAA suite of programs, including, but not limited to, PicXAA, PicXAA-R, PicXAA-Web, etc. (Mohammad, S., Sahraeian, E. & Yoon, B. PicXAA: greedy probabilistic construction of maximum expected accuracy alignment of multiple sequences. Nucleic Acids Research. 38(15):4917-28. 2010). In an embodiment, the subject disclosure relates to calculating percent identity between two polynucleotides or amino acid sequences using the PSAlign alignment program (SZE, S.-H., Lu, Y., & Yang, Q. (2006) A polynomial time solvable formulation of multiple sequence alignment Journal of Computational Biology, 13, 309-19). In an embodiment, the subject disclosure relates to calculating percent identity between two polynucleotides or amino acid sequences using the StatAlign alignment program (Novak, A., et. al. (2008) StatAlign: an extendable software package for joint Bayesian estimation of alignments and evolutionary trees. Bioinformatics, 24(20):2403-04). In an embodiment, the subject disclosure relates to calculating percent identity between two polynucleotides or amino acid sequences using the Gap alignment program of Needleman and Wunsch (Needleman and Wunsch, Journal of Molecular Biology 48:443-453, 1970). In an embodiment, the subject disclosure relates to calculating percent identity between two polynucleotides or amino acid sequences using the BestFit alignment program of Smith and Waterman (Smith and Waterman, Advances in Applied Mathematics, 2:482-489, 1981, Smith et al., Nucleic Acids Research 11:2205-2220, 1983). These programs produces biologically meaningful multiple sequence alignments of divergent sequences. The calculated best match alignments for the selected sequences are lined up so that identities, similarities, and differences can be seen. The following abbreviations are produced during a BLAST analysis of a sequence.

| | |
|---|---|
| SEQ_NUM | provides the SEQ ID NO for the listed recombinant |
| | polynucleotide sequences. |
| CONTIG_ID | provides an arbitrary sequence name taken from the name of the |
| | clone from which the cDNA sequence was obtained. |
| PROTEIN_NUM | provides the SEQ ID NO for the recombinant polypeptide |
| | sequence |
| NCBI_GI | provides the GenBank ID number for the top BLAST hit for the |
| | sequence. The top BLAST hit is indicated by the National Center |
| | for Biotechnology Information GenBank Identifier number. |
| NCBI_GI_DESCRIPTION | refers to the description of the GenBank top BLAST hit for the |
| | sequence. |
| E_VALUE | provides the expectation value for the top BLAST match. |
| MATCH_LENGTH | provides the length of the sequence which is aligned in the top |
| | BLAST match |
| TOP_HIT_PCT_IDENT | refers to the percentage of identically matched nucleotides (or |
| | residues) that exist along the length of that portion of the |
| | sequences which is aligned in the top BLAST match. |
| CAT_TYPE | indicates the classification scheme used to classify the sequence. |
| | GO_BP = Gene |
| | Ontology Consortium - biological process; GO_CC = Gene |
| | Ontology Consortium - |
| | cellular component; GO_MF = Gene Ontology Consortium - |
| | molecular function; KEGG = KEGG |
| | functional hierarchy (KEGG = Kyoto Encyclopedia of Genes |
| | and Genomes); |
| | EC = Enzyme Classification from ENZYME data bank release |
| | 25.0; POI = Pathways of Interest. |
| CAT_DESC | provides the classification scheme subcategory to which the |
| | query sequence was assigned. |
| PRODUCT_CAT_DESC | provides the FunCAT annotation category to which the query |
| | sequence was assigned. |
| PRODUCT_HIT_DESC | provides the description of the BLAST hit which resulted in |
| | assignment of the sequence to the function category provided in |
| | the cat_desc column. |
| HIT_E | provides the E value for the BLAST hit in the hit_desc column. |
| PCT_IDENT | refers to the percentage of identically matched nucleotides (or |
| | residues) that exist along the length of that portion of the |
| | sequences which is aligned in the BLAST match provided in |
| | hit_desc. |
| QRY_RANGE | lists the range of the query sequence aligned with the hit. |
| HIT_RANGE | lists the range of the hit sequence aligned with the query. |
| QRY_CVRG | provides the percent of query sequence length that matches to |
| | the hit (NCBI). |
| | sequence in the BLAST match (% qry cvrg = (match |
| | length/query total length) × 100). |
| HIT_CVRG | provides the percent of hit sequence length that matches to the |
| | query sequence in the match generated using BLAST (% hit |
| | cvrg = (match length/hit total length) × 100). |

The term “similarity” refers to a comparison between amino acid sequences, and takes into account not only identical amino acids in corresponding positions, but also functionally similar amino acids in corresponding positions. Thus similarity between polypeptide sequences indicates functional similarity, in addition to sequence similarity.

The term “homology” is sometimes used to refer to the level of similarity between two or more nucleic acid or amino acid sequences in terms of percent of positional identity (i.e., sequence similarity or identity). Homology also refers to the concept of evolutionary relatedness, often evidenced by similar functional properties among different nucleic acids or proteins that share similar sequences.

As used herein, the term “variants” means substantially similar sequences. For nucleotide sequences, naturally occurring variants can be identified with the use of well-known molecular biology techniques, such as, for example, with polymerase chain reaction (PCR) and hybridization techniques as outlined herein.

For nucleotide sequences, a variant comprises a deletion and/or addition of one or more nucleotides at one or more internal sites within the native polynucleotide and/or a substitution of one or more nucleotides at one or more sites in the native polynucleotide. As used herein, a “native” nucleotide sequence comprises a naturally occurring nucleotide sequence. For nucleotide sequences, naturally occurring variants can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis. Generally, variants of a particular nucleotide sequence of the disclosure will have at least about 40%, 45%, 50%, 55%, 60%, 65%, 70%, 75%, 80%, 85%, 90%, 91%, 92%, 93%, 94%, 95%, 96%, 97%, 98% o, 99% or more sequence identity to that particular nucleotide sequence as determined by sequence alignment programs and parameters described elsewhere herein. A biologically active variant of a nucleotide sequence of the disclosure may differ from that sequence by as few as 1-15 nucleic acid residues, as few as 1-10, such as 6-10, as few as 5, as few as 4, 3, 2, or even 1 nucleic acid residue.

As used herein the term “operably linked” relates to a first nucleic acid sequence that is operably linked with a second nucleic acid sequence when the first nucleic acid sequence is in a functional relationship with the second nucleic acid sequence. For instance, a promoter is operably linked with a coding sequence when the promoter affects the transcription or expression of the coding sequence. When recombinantly produced, operably linked nucleic acid sequences are generally contiguous and, where necessary to join two protein-coding regions, in the same reading frame. However, elements need not be contiguous to be operably linked.

As used herein, the term “orally active” refers to a molecule that inhibits the proliferation of insect pests when orally ingested by the insect pest.

As used herein, the term “insecticidal activity” refers to activity of an organism or a substance (such as, for example, a protein) that can be measured by, but is not limited to, insect mortality, insect weight loss, reduced reproduction, insect repellency, and other behavioral and physical changes of an insect after feeding and exposure for an appropriate length of time. Thus, an organism or substance having insecticidal activity adversely impacts at least one measurable parameter of insect fitness.

An “open reading frame” is a continuous stretch of DNA beginning with a start codon (e.g., methionine (ATG)), and ending with a stop codon (e.g., TAA, TAG or TGA) and encodes a protein or peptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

As used herein, the term “pest” refers to any insect that is unwanted and disruptive or destructive to the growth and development of agricultural crops. The term “insect pest” includes but is not limited to, insects, fungi, bacteria, nematodes, mites, ticks, and the like. Insect pests include insects selected from the orders Coleoptera, Diptera, Hymenoptera, Lepidoptera, Mallophaga, Homoptera, Hemiptera, Thysanoptera, Dermaptera, Isoptera, Anoplura, Siphonaptera, Trichoptera, etc., particularly Lepidoptera, and Hemiptera.

As used herein, the term “stable transformation” or “stably transformed” is intended to mean that the nucleotide construct introduced into a plant integrates into the genome of the plant and is capable of being inherited by the progeny thereof “Transient transformation” is intended to mean that a polynucleotide is introduced into the plant and does not integrate into the genome of the plant or a polypeptide is introduced into a plant. By “plant” is intended whole plants, plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, and pollen).

As used herein, the term “regeneration” means the process of growing a plant from a plant cell (e.g., plant protoplast or explant).

As used herein, the term “culturing” refers to the in vitro propagation of cells or organisms on or in media of various kinds so that the maintenance or growth of cells within a liquid culture medium are controlled under a set of physical conditions. It is understood that the descendants of a cell grown in culture may not be completely identical (i.e., morphologically, genetically, or phenotypically) to the parent cell.

As used herein, the term “controlling” (for instance as in “controlling an insect pest population”), refers to monitoring, treating, minimizing, exterminating, or preventing insect pests such as stink bugs. In specific instances the insect species are controlled to reducing the number of insects that cause reduced beneficial plant yield.

As used herein, the term “insecticidally-effective amount” refers to a quantity of a substance or organism that has insecticidal activity when present in the environment of an insect pest. For each substance or organism, the insecticidally-effective amount is determined empirically for each pest affected in a specific environment. Similarly, an “pesticidally effective amount” may be used to refer to an insecticidally-effective amount.

As used herein, the term “pesticidal protein” or “insecticidal protein” is intended to refer to a polypeptide that has toxic activity against one or more pests, including, but not limited to, members of the Lepidoptera, Diptera, Hemiptera and Coleoptera orders or the Nematoda phylum or a protein that has homology to such a protein. Pesticidal proteins have been isolated from organisms including, for example, Bacillus sp., Pseudomonas sp., Photorhabdus sp., Xenorhabdus sp., Clostridium bifermentans and Paenibacillus popilliae. Pesticidal proteins include but are not limited to: insecticidal proteins from Pseudomonas sp. such as PSEEN3174 (Monalysin, (2011) PLoS Pathogens, 7:1-13), from Pseudomonas protegens strain CHA0 and Pf-5 (previously fluorescens) (Pechy-Tarr, (2008) Environmental Microbiology 10:2368-2386: GenBank Accession No. EU400157); from Pseudomonas Taiwanensis (Liu, et al., (2010) J. Agric. Food Chem. 58:12343-12349) and from Pseudomonas pseudoalcligenes (Zhang, et al., (2009) Annals of Microbiology 59:45-50 and Li, et al., (2007) Plant Cell Tiss. Organ Cult. 89:159-168); insecticidal proteins from Photorhabdus sp. and Xenorhabdus sp. (Hinchliffe, et al., (2010) The Open Toxinology Journal 3:101-118 and Morgan, et al., (2001) Applied and Envir. Micro. 67:2062-2069), U.S. Pat. Nos. 6,048,838, and 6,379,946; and δ-endotoxins including, but not limited to, the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry 51 and Cry55 classes of δ-endotoxin genes and the B. thuringiensis cytolytic Cyt1 and Cyt2 genes. Members of these classes of B. thuringiensis insecticidal proteins include, but are not limited to Cry1Aa1 (Accession #Accession #M11250), Cry1Aa2 (Accession #M10917), Cry1Aa3 (Accession #D00348), Cry1Aa4 (Accession #X13535), Cry1Aa5 (Accession #D17518), Cry1Aa6 (Accession #U43605), Cry1Aa7 (Accession #AF081790), Cry1Aa8 (Accession #I26149), Cry1Aa9 (Accession #AB026261), Cry1Aa10 (Accession #AF154676), Cry1Aa11 (Accession #Y09663), Cry1Aa12 (Accession #AF384211), Cry1Aa13 (Accession #AF510713), Cry1Aa14 (Accession #AY197341), Cry1Aa15 (Accession #DQ062690), Cry1Ab1 (Accession #M13898), Cry1Ab2 (Accession #M12661), Cry1Ab3 (Accession #M15271), Cry1Ab4 (Accession #D00117), Cry1Ab5 (Accession #X04698), Cry1Ab6 (Accession #M37263), Cry1Ab7 (Accession #X13233), Cry1Ab8 (Accession #M16463), Cry1Ab9 (Accession #X54939), Cry1Ab10 (Accession #A29125), Cry1Ab 11 (Accession #I12419), Cry1Ab12 (Accession #AF059670), Cry1Ab13 (Accession #AF254640), Cry1Ab14 (Accession #U94191), Cry1Ab15 (Accession #AF358861), Cry1Ab16 (Accession #AF375608), Cry1Ab17 (Accession #AAT46415), Cry1Ab18 (Accession #AAQ88259), Cry1Ab19 (Accession #AY847289), Cry1Ab20 (Accession #DQ241675), Cry1Ab21 (Accession #EF683163), Cry1Ab22 (Accession #ABW87320), Cry1Ab-like (Accession #AF327924), Cry1Ab-like (Accession #AF327925), Cry1Ab-like (Accession #AF327926), Cry1Ab-like (Accession #DQ781309), Cry1Ac1 (Accession #M11068), Cry1Ac2 (Accession #M35524), Cry1Ac3 (Accession #X54159), Cry1Ac4 (Accession #M73249), Cry1Ac5 (Accession #M73248), Cry1Ac6 (Accession #U43606), Cry1Ac7 (Accession #U87793), Cry1Ac8 (Accession #U87397), Cry1Ac9 (Accession #U89872), Cry1Ac10 (Accession #AJ002514), Cry1Ac11 (Accession #AJ130970), Cry1Ac12 (Accession #I12418), Cry1Ac13 (Accession #AF148644), Cry1Ac14 (Accession #AF492767), Cry1Ac15 (Accession #AY122057), Cry1Ac16 (Accession #AY730621), Cry1Ac17 (Accession #AY925090), Cry1Ac18 (Accession #DQ023296), Cry1Ac19 (Accession #DQ195217), Cry1Ac20 (Accession #DQ285666), Cry1Ac21 (Accession #DQ062689), Cry1Ac22 (Accession #EU282379), Cry1Ac23 (Accession #AM949588), Cry1Ac24 (Accession #ABL01535), Cry1Ad1 (Accession #M73250), Cry1Ad2 (Accession #A27531), Cry1Ae1 (Accession #M65252), Cry1Af1 (Accession #U82003), Cry1Ag1 (Accession #AF081248), Cry1Ah1 (Accession #AF281866), Cry1Ah2 (Accession #DQ269474), Cry1Ai1 (Accession #AY174873), Cry1A-like (Accession #AF327927), Cry1Ba1 (Accession #X06711), Cry1Ba2 (Accession #X95704), Cry1Ba3 (Accession #AF368257), Cry1Ba4 (Accession #AF363025), Cry1Ba5 (Accession #AB020894), Cry1Ba6 (Accession #ABL60921), Cry1Bb1 (Accession #L32020), Cry1Bc1 (Accession #Z46442), Cry1Bd1 (Accession #U70726), Cry1Bd2 (Accession #AY138457), Cry1Be1 (Accession #AF077326), Cry1Be2 (Accession #AAQ52387), Cry1Bf1 (Accession #AX189649), Cry1Bf2 (Accession #AAQ52380), Cry1Bg1 (Accession #AY176063), Cry1Ca1 (Accession #X07518), Cry1Ca2 (Accession #X13620), Cry1Ca3 (Accession #M73251), Cry1Ca4 (Accession #A27642), Cry1Ca5 (Accession #X96682), Cry1Ca6 [1] (Accession #AF215647), Cry1Ca7 (Accession #AY015492), Cry1 Cab (Accession #AF362020), Cry1Ca9 (Accession #AY078160), Cry1Ca10 (Accession #AF540014), Cry1Ca11 (Accession #AY955268), Cry1Cb1 (Accession #M97880), Cry1Cb2 (Accession #AY007686), Cry1Cb3 (Accession #EU679502), Cry1 Cb-like (Accession #AAX63901), Cry1Da1 (Accession #X54160), Cry1Da2 (Accession #I76415), Cry1db1 (Accession #Z22511), Cry1db2 (Accession #AF358862), Cry1Dc1 (Accession #EF059913), Cry1 Eat (Accession #X53985), Cry1Ea2 (Accession #X56144), Cry1Ea3 (Accession #M73252), Cry1Ea4 (Accession #U94323), Cry1Ea5 (Accession #A15535), Cry1Ea6 (Accession #AF202531), Cry1Ea7 (Accession #AAW72936), Cry1Ea8 (Accession #ABX11258), Cry1Eb1 (Accession #M73253), Cry1Fa1 (Accession #M63897), Cry1Fa2 (Accession #M73254), Cry1Fb1 (Accession #Z22512), Cry1Fb2 (Accession #AB012288), Cry1Fb3 (Accession #AF062350), Cry1Fb4 (Accession #I73895), Cry1Fb5 (Accession #AF336114), Cry1Fb6 (Accession #EU679500), Cry1Fb7 (Accession #EU679501), Cry1Ga1 (Accession #Z22510), Cry1Ga2 (Accession #Y09326), Cry1Gb1 (Accession #U70725), Cry1Gb2 (Accession #AF288683), Cry1Gc (Accession #AAQ52381), Cry1Ha1 (Accession #Z22513), Cry1Hb1 (Accession #U35780), Cry1H-like (Accession #AF182196), Cry1Ia1 (Accession #X62821), Cry1Ia2 (Accession #M98544), Cry1Ia3 (Accession #L36338), Cry1Ia4 (Accession #L49391), Cry1Ia5 (Accession #Y08920), Cry1Ia6 (Accession #AF076953), Cry1Ia7 (Accession #AF278797), Cry1Ia8 (Accession #AF373207), Cry1Ia9 (Accession #AF521013), Cry1Ia10 (Accession #AY262167), Cry1Ia11 (Accession #AJ315121), Cry1Ia12 (Accession #AAV53390), Cry1Ia13 (Accession #ABF83202), Cry1Ia14 (Accession #EU887515), Cry1Ib1 (Accession #U07642), Cry1Ib2 (Accession #ABW88019), Cry1Ib3 (Accession #EU677422), Cry1Ic1 (Accession #AF056933), Cry1Ic2 (Accession #AAE71691), Cry1Id1 (Accession #AF047579), Cry1Ie1 (Accession #AF211190), Cry1If1 (Accession #AAQ52382), Cry1I-like (Accession #I90732), Cry1I-like (Accession #DQ781310), Cry1Ja1 (Accession #L32019), Cry1Jb1 (Accession #U31527), Cry1Jc1 (Accession #I90730), Cry1Jc2 (Accession #AAQ52372), Cry1Jd1 (Accession #AX189651), Cry1 Kat (Accession #U28801), Cry1La1 (Accession #AAS60191), Cry1-like (Accession #190729), Cry2Aa1 (Accession #M31738), Cry2Aa2 (Accession #M23723), Cry2Aa3 (Accession #D86064), Cry2Aa4 (Accession #AF047038), Cry2Aa5 (Accession #AJ132464), Cry2Aa6 (Accession #AJ132465), Cry2Aa7 (Accession #AJ132463), Cry2Aa8 (Accession #AF252262), Cry2Aa9 (Accession #AF273218), Cry2Aa10 (Accession #AF433645), Cry2Aa11 (Accession #AAQ52384), Cry2Aa12 (Accession #DQ977646), Cry2Aa13 (Accession #ABL01536), Cry2Aa14 (Accession #ACF04939), Cry2Ab1 (Accession #M23724), Cry2Ab2 (Accession #X55416), Cry2Ab3 (Accession #AF164666), Cry2Ab4 (Accession #AF336115), Cry2Ab5 (Accession #AF441855), Cry2Ab6 (Accession #AY297091), Cry2Ab7 (Accession #DQ119823), Cry2Ab8 (Accession #DQ361266), Cry2Ab9 (Accession #DQ341378), Cry2Ab10 (Accession #EF157306), Cry2Ab11 (Accession #AM691748), Cry2Ab12 (Accession #ABM21764), Cry2Ab13 (Accession #EU909454), Cry2Ab14 (Accession #EU909455), Cry2Ac1 (Accession #X57252), Cry2Ac2 (Accession #AY007687), Cry2Ac3 (Accession #AAQ52385), Cry2Ac4 (Accession #DQ361267), Cry2Ac5 (Accession #DQ341379), Cry2Ac6 (Accession #DQ359137), Cry2Ac7 (Accession #AM292031), Cry2Ac8 (Accession #AM421903), Cry2Ac9 (Accession #AM421904), Cry2Ac10 (Accession #BI 877475), Cry2Ac11 (Accession #AM689531), Cry2Ac12 (Accession #AM689532), Cry2Ad1 (Accession #AF200816), Cry2Ad2 (Accession #DQ358053), Cry2Ad3 (Accession #AM268418), Cry2Ad4 (Accession #AM490199), Cry2Ad5 (Accession #AM765844), Cry2Ae1 (Accession #AAQ52362), Cry2Af1 (Accession #EF439818), Cry2Ag (Accession #ACH91610), Cry2Ah (Accession #EU939453), Cry3Aa1 (Accession #M22472), Cry3Aa2 (Accession #J02978), Cry3Aa3 (Accession #Y00420), Cry3Aa4 (Accession #M30503), Cry3Aa5 (Accession #M37207), Cry3Aa6 (Accession #U10985), Cry3Aa7 (Accession #AJ237900), Cry3Aa8 (Accession #AAS79487), Cry3Aa9 (Accession #AAW05659), Cry3Aa10 (Accession #AAU29411), Cry3Aa11 (Accession #AY882576), Cry3Aa12 (Accession #ABY49136), Cry3Ba1 (Accession #X17123), Cry3Ba2 (Accession #A07234), Cry3Bb1 (Accession #M89794), Cry3Bb2 (Accession #U31633), Cry3Bb3 (Accession #I15475), Cry3Ca1 (Accession #X59797), Cry4Aa1 (Accession #Y00423), Cry4Aa2 (Accession #D00248), Cry4Aa3 (Accession #AL731825), Cry4A-like (Accession #DQ078744), Cry4Ba1 (Accession #X07423), Cry4Ba2 (Accession #X07082), Cry4Ba3 (Accession #M20242), Cry4Ba4 (Accession #D00247), Cry4Ba5 (Accession #AL731825), Cry4Ba-like (Accession #ABC47686), Cry4Ca1 (Accession #EU646202), Cry5Aa1 (Accession #L07025), Cry5Ab1 (Accession #L07026), Cry5Ac1 (Accession #I34543), Cry5Ad1 (Accession #EF219060), Cry5Ba1 (Accession #U19725), Cry5Ba2 (Accession #EU121522), Cry6Aa1 (Accession #L07022), Cry6Aa2 (Accession #AF499736), Cry6Aa3 (Accession #DQ835612), Cry6Ba1 (Accession #L07024), Cry7Aa1 (Accession #M64478), Cry7Ab1 (Accession #U04367), Cry7Ab2 (Accession #U04368), Cry7Ab3 (Accession #BI 1015188), Cry7Ab4 (Accession #EU380678), Cry7Ab5 (Accession #ABX79555), Cry7Ab6 (Accession #FJ194973), Cry7Ba1 (Accession #ABB70817), Cry7Ca1 (Accession #EF486523), Cry8Aa1 (Accession #U04364), Cry8Ab1 (Accession #EU044830), Cry8Ba1 (Accession #U04365), Cry8Bb1 (Accession #AX543924), Cry8Bc1 (Accession #AX543926), Cry8Ca1 (Accession #U04366), Cry8Ca2 (Accession #AAR98783), Cry8Ca3 (Accession #EU625349), Cry8Da1 (Accession #AB089299), Cry8Da2 (Accession #BD133574), Cry8Da3 (Accession #BD133575), Cry8 db1 (Accession #AB303980), Cry8Ea1 (Accession #AY329081), Cry8Ea2 (Accession #EU047597), Cry8Fa1 (Accession #AY551093), Cry8Ga1 (Accession #AY590188), Cry8Ga2 (Accession #DQ318860), Cry8Ga3 (Accession #FJ198072), Cry8Ha1 (Accession #EF465532), Cry8Ia1 (Accession #EU381044), Cry8Ja1 (Accession #EU625348), Cry8 like (Accession #ABS53003), Cry9Aa1 (Accession #X58120), Cry9Aa2 (Accession #X58534), Cry9Aa like (Accession #AAQ52376), Cry9Ba1 (Accession #X75019), Cry9Bb1 (Accession #AY758316), Cry9Ca1 (Accession #Z37527), Cry9Ca2 (Accession #AAQ52375), Cry9Da1 (Accession #D85560), Cry9Da2 (Accession #AF042733), Cry9 db1 (Accession #AY971349), Cry9Ea1 (Accession #AB011496), Cry9Ea2 (Accession #AF358863), Cry9Ea3 (Accession #EF157307), Cry9Ea4 (Accession #EU760456), Cry9Ea5 (Accession #EU789519), Cry9Ea6 (Accession #EU887516), Cry9Eb1 (Accession #AX189653), Cry9Ec1 (Accession #AF093107), Cry9Ed1 (Accession #AY973867), Cry9 like (Accession #AF093107), Cry10Aa1 (Accession #M12662), Cry10Aa2 (Accession #E00614), Cry10Aa3 (Accession #AL731825), Cry10A like (Accession #DQ167578), Cry11Aa1 (Accession #M31737), Cry11Aa2 (Accession #M22860), Cry11Aa3 (Accession #AL731825), Cry11Aa-like (Accession #DQ166531), Cry11Ba1 (Accession #X86902), Cry11Bb1 (Accession #AF017416), Cry12Aa1 (Accession #L07027), Cry13Aa1 (Accession #L07023), Cry14Aa1 (Accession #U13955), Cry15Aa1 (Accession #M76442), Cry16Aa1 (Accession #X94146), Cry17Aa1 (Accession #X99478), Cry18Aa1 (Accession #X99049), Cry18Ba1 (Accession #AF169250), Cry18Ca1 (Accession #AF169251), Cry19Aa1 (Accession #Y07603), Cry19Ba1 (Accession #D88381), Cry20Aa1 (Accession #U82518), Cry21Aa1 (Accession #I32932), Cry21Aa2 (Accession #I66477), Cry21Ba1 (Accession #AB088406), Cry22Aa1 (Accession #I34547), Cry22Aa2 (Accession #AX472772), Cry22Aa3 (Accession #EU715020), Cry22Ab1 (Accession #AAK50456), Cry22Ab2 (Accession #AX472764), Cry22Ba1 (Accession #AX472770), Cry23Aa1 (Accession #AAF76375), Cry24Aa1 (Accession #U88188), Cry24Ba1 (Accession #BAD32657), Cry24Ca1 (Accession #AM158318), Cry25Aa1 (Accession #U88189), Cry26Aa1 (Accession #AF122897), Cry27Aa1 (Accession #AB023293), Cry28Aa1 (Accession #AF132928), Cry28Aa2 (Accession #AF285775), Cry29Aa1 (Accession #AJ251977), Cry30Aa1 (Accession #AJ251978), Cry30Ba1 (Accession #BAD00052), Cry30Ca1 (Accession #BAD67157), Cry30Da1 (Accession #EF095955), Cry30 db1 (Accession #BAE80088), Cry30Ea1 (Accession #EU503140), Cry30Fa1 (Accession #EU751609), Cry30Ga1 (Accession #EU882064), Cry31Aa1 (Accession #AB031065), Cry31Aa2 (Accession #AY081052), Cry31Aa3 (Accession #AB250922), Cry31Aa4 (Accession #AB274826), Cry31Aa5 (Accession #AB274827), Cry31Ab1 (Accession #AB250923), Cry31Ab2 (Accession #AB274825), Cry31Ac1 (Accession #AB276125), Cry32Aa1 (Accession #AY008143), Cry32Ba1 (Accession #BAB78601), Cry32Ca1 (Accession #BAB78602), Cry32Da1 (Accession #BAB78603), Cry33Aa1 (Accession #AAL26871), Cry34Aa1 (Accession #AAG50341), Cry34Aa2 (Accession #AAK64560), Cry34Aa3 (Accession #AY536899), Cry34Aa4 (Accession #AY536897), Cry34Ab1 (Accession #AAG41671), Cry34Ac1 (Accession #AAG50118), Cry34Ac2 (Accession #AAK64562), Cry34Ac3 (Accession #AY536896), Cry34Ba1 (Accession #AAK64565), Cry34Ba2 (Accession #AY536900), Cry34Ba3 (Accession #AY536898), Cry35Aa1 (Accession #AAG50342), Cry35Aa2 (Accession #AAK64561), Cry35Aa3 (Accession #AY536895), Cry35Aa4 (Accession #AY536892), Cry35Ab1 (Accession #AAG41672), Cry35Ab2 (Accession #AAK64563), Cry35Ab3 (Accession #AY536891), Cry35Ac1 (Accession #AAG50117), Cry35Ba1 (Accession #AAK64566), Cry35Ba2 (Accession #AY536894), Cry35Ba3 (Accession #AY536893), Cry36Aa1 (Accession #AAK64558), Cry37Aa1 (Accession #AAF76376), Cry38Aa1 (Accession #AAK64559), Cry39Aa1 (Accession #BAB72016), Cry40Aa1 (Accession #BAB72018), Cry40Ba1 (Accession #BAC77648), Cry40Ca1 (Accession #EU381045), Cry40Da1 (Accession #EU596478), Cry41Aa1 (Accession #AB 116649), Cry41Ab1 (Accession #AB116651), Cry42Aa1 (Accession #AB116652), Cry43Aa1 (Accession #AB115422), Cry43Aa2 (Accession #AB176668), Cry43Ba1 (Accession #AB115422), Cry43-like (Accession #AB115422), Cry44Aa (Accession #BAD08532), Cry45Aa (Accession #BAD22577), Cry46Aa (Accession #BAC79010), Cry46Aa2 (Accession #BAG68906), Cry46Ab (Accession #BAD35170), Cry47Aa (Accession #AY950229), Cry48Aa (Accession #AJ841948), Cry48Aa2 (Accession #AM237205), Cry48Aa3 (Accession #AM237206), Cry48Ab (Accession #AM237207), Cry48Ab2 (Accession #AM237208), Cry49Aa (Accession #AJ841948), Cry49Aa2 (Accession #AM237201), Cry49Aa3 (Accession #AM237203), Cry49Aa4 (Accession #AM237204), Cry49Ab1 (Accession #AM237202), Cry50Aa1 (Accession #AB253419), Cry51Aa1 (Accession #DQ836184), Cry52Aa1 (Accession #EF613489), Cry53Aa1 (Accession #EF633476), Cry54Aa1 (Accession #EU339367), Cry55Aa1 (Accession #EU121521), Cry55Aa2 (Accession #AAE33526).

Examples of δ-endotoxins also include but are not limited to Cry1A proteins of U.S. Pat. Nos. 5,880,275 and 7,858,849; a DIG-3 or DIG-11 toxin (N-terminal deletion of a-helix 1 and/or a-helix 2 variants of Cry proteins such as Cry1A) of U.S. Pat. Nos. 8,304,604 and 8,304,605, Cry1B of U.S. patent application Ser. No. 10/525,318; Cry1C of U.S. Pat. No. 6,033,874; Cry1F of U.S. Pat. Nos. 5,188,960, 6,218,188; Cry1A/F chimeras of U.S. Pat. Nos. 7,070,982; 6,962,705 and 6,713,063); a Cry2 protein such as Cry2Ab protein of U.S. Pat. No. 7,064,249); a Cry3A protein including but not limited to an engineered hybrid insecticidal protein (eHIP) created by fusing unique combinations of variable regions and conserved blocks of at least two different Cry proteins (US Patent Application Publication Number 2010/0017914); a Cry4 protein; a Cry5 protein; a Cry6 protein; Cry8 proteins of U.S. Pat. Nos. 7,329,736, 7,449,552, 7,803,943, 7,476,781, 7,105,332, 7,378,499 and 7,462,760; a Cry9 protein such as such as members of the Cry9A, Cry9B, Cry9C, Cry9D, Cry9E, and Cry9F families; a Cry15 protein of Naimov, et al., (2008) Applied and Environmental Microbiology 74:7145-7151; a Cry22, a Cry34Ab1 protein of U.S. Pat. Nos. 6,127,180, 6,624,145 and 6,340,593; a CryET33 and CryET34 protein of U.S. Pat. Nos. 6,248,535, 6,326,351, 6,399,330, 6,949,626, 7,385,107 and 7,504,229; a CryET33 and CryET34 homologs of US Patent Publication Number 2006/0191034, 2012/0278954, and PCT Publication Number WO 2012/139004; a Cry35Ab1 protein of U.S. Pat. Nos. 6,083,499, 6,548,291 and 6,340,593; a Cry46 protein, a Cry 51 protein, a Cry binary toxin; a TIC901 or related toxin; TIC807 of US 2008/0295207; ET29, ET37, TIC809, TIC810, TIC812, TIC127, TIC128 of PCT US 2006/033867; AXMI-027, AXMI-036, and AXMI-038 of U.S. Pat. No. 8,236,757; AXMI-031, AXMI-039, AXMI-040, AXMI-049 of U.S. Pat. No. 7,923,602; AXMI-018, AXMI-020, and AXMI-021 of WO 2006/083891; AXMI-010 of WO 2005/038032; AXMI-003 of WO 2005/021585; AXMI-008 of US 2004/0250311; AXMI-006 of US 2004/0216186; AXMI-007 of US 2004/0210965; AXMI-009 of US 2004/0210964; AXMI-014 of US 2004/0197917; AXMI-004 of US 2004/0197916; AXMI-028 and AXMI-029 of WO 2006/119457; AXMI-007, AXMI-008, AXMI-0080r12, AXMI-009, AXMI-014 and AXMI-004 of WO 2004/074462; AXMI-150 of U.S. Pat. No. 8,084,416; AXMI-205 of US20110023184; AXMI-011, AXMI-012, AXMI-013, AXMI-015, AXMI-019, AXMI-044, AXMI-037, AXMI-043, AXMI-033, AXMI-034, AXMI-022, AXMI-023, AXMI-041, AXMI-063, and AXMI-064 of US 2011/0263488; AXMI-R1 and related proteins of US 2010/0197592; AXMI221Z, AXMI222z, AXMI223z, AXMI224z and AXMI225z of WO 2011/103248; AXMI218, AXMI219, AXMI220, AXMI226, AXMI227, AXMI228, AXMI229, AXMI230, and AXMI231 of WO11/103,247; AXMI-115, AXMI-113, AXMI-005, AXMI-163 and AXMI-184 of U.S. Pat. No. 8,334,431; AXMI-001, AXMI-002, AXMI-030, AXMI-035, and AXMI-045 of US 2010/0298211; AXMI-066 and AXMI-076 of US20090144852; AXMI128, AXMI130, AXMI131, AXMI133, AXMI140, AXMI141, AXMI142, AXMI143, AXMI144, AXMI146, AXMI148, AXMI149, AXMI152, AXMI153, AXMI154, AXMI155, AXMI156, AXMI157, AXMI158, AXMI162, AXMI165, AXMI166, AXMI167, AXMI168, AXMI169, AXMI170, AXMI171, AXMI172, AXMI173, AXMI174, AXMI175, AXMI176, AXMI177, AXMI178, AXMI179, AXMI180, AXMI181, AXMI182, AXMI185, AXMI186, AXMI187, AXMI188, AXMI189 of U.S. Pat. No. 8,318,900; AXMI079, AXMI080, AXMI081, AXMI082, AXMI091, AXMI092, AXMI096, AXMI097, AXMI098, AXMI099, AXMI100, AXMI101, AXMI102, AXMI103, AXMI104, AXMI107, AXMI108, AXMI109, AXMI110, AXMI111, AXMI112, AXMI114, AXMI116, AXMI117, AXMI118, AXMI119, AXMI120, AXMI121, AXMI122, AXMI123, AXMI124, AXMI1257, AXMI1268, AXMI127, AXMI129, AXMI164, AXMI151, AXMI161, AXMI183, AXMI132, AXMI138, AXMI137 of US 2010/0005543; Cry proteins such as Cry1A and Cry3A having modified proteolytic sites of U.S. Pat. No. 8,319,019; and a Cry1Ac, Cry2Aa and Cry1Ca toxin protein from Bacillus thuringiensis strain VBTS 2528 of US Patent Application Publication Number 2011/0064710. Other Cry proteins are well-known to one skilled in the art (see, Crickmore, et al., “Bacillus thuringiensis toxin nomenclature” (2011), at lifesci.sussex.ac.uk/home/Neil_Crickmore/Bt/which can be accessed on the world-wide web using the “www” prefix). The insecticidal activity of Cry proteins is well-known to one skilled in the art (for review, see, van Frannkenhuyzen, (2009) J. Invert. Path. 101:1-16). The use of Cry proteins as transgenic plant traits is well-known to one skilled in the art and Cry-transgenic plants including but not limited to Cry1Ac, Cry1Ac+Cry2Ab, Cry1Ab, Cry1A.105, Cry1F, Cry1Fa2, Cry1F+Cry1Ac, Cry2Ab, Cry3A, mCry3A, Cry3Bb1, Cry34Ab1, Cry35Ab1, Vip3A, mCry3A, Cry9c and CBI-Bt have received regulatory approval (see, Sanahuja, (2011) Plant Biotech Journal 9:283-300 and the CERA (2010) GM Crop Database Center for Environmental Risk Assessment (CERA), ILSI Research Foundation, Washington D.C. at cera-gmc.org/index.php?action=gm_crop_database which can be accessed on the world-wide web using the “www” prefix). More than one pesticidal proteins well-known to one skilled in the art can also be expressed in plants such as Vip3Ab & Cry1Fa (US2012/0317682), Cry1BE & Cry1F (US2012/0311746), Cry1CA & Cry1AB (US2012/0311745), Cry1F & CryCa (US2012/0317681), Cry1DA & Cry1BE (US2012/0331590), Cry1DA & Cry1Fa (US2012/0331589), Cry1AB & Cry1BE (US2012/0324606), and Cry1Fa & Cry2Aa, Cry1I or Cry1E (US2012/0324605). Pesticidal proteins also include insecticidal lipases including lipid acyl hydrolases of U.S. Pat. No. 7,491,869, and cholesterol oxidases such as from Streptomyces (Purcell et al. (1993) Biochem Biophys Res Commun 15:1406-1413). Pesticidal proteins also include VIP (vegetative insecticidal proteins) toxins of U.S. Pat. Nos. 5,877,012, 6,107,279, 6,137,033, 7,244,820, 7,615,686, and 8,237,020, and the like. Other VIP proteins are well-known to one skilled in the art (see, lifesci.sussex.ac.uk/home/Neil_Crickmore/Bt/vip.html which can be accessed on the world-wide web using the “www” prefix). Pesticidal proteins also include toxin complex (TC) proteins, obtainable from organisms such as Xenorhabdus, Photorhabdus and Paenibacillus (see, U.S. Pat. Nos. 7,491,698 and 8,084,418). Some TC proteins have “stand-alone” insecticidal activity and other TC proteins enhance the activity of the stand-alone toxins produced by the same given organism. The toxicity of a “stand-alone” TC protein (from Photorhabdus, Xenorhabdus or Paenibacillus, for example) can be enhanced by one or more TC protein “potentiators” derived from a source organism of a different genus. There are three main types of TC proteins. As referred to herein, Class A proteins (“Protein A”) are stand-alone toxins. Class B proteins (“Protein B”) and Class C proteins (“Protein C”) enhance the toxicity of Class A proteins. Examples of Class A proteins are TcbA, TcdA, XptA1 and XptA2. Examples of Class B proteins are TcaC, TcdB, XptB1Xb and XptC1Wi. Examples of Class C proteins are TccC, XptC1Xb and XptB1Wi. Pesticidal proteins also include spider, snake and scorpion venom proteins. Examples of spider venom peptides include but are not limited to lycotoxin-1 peptides and mutants thereof (U.S. Pat. No. 8,334,366). Further examples include IPD072 (PCT/US14/55128), and IPD079 (PCT/US2016/041452).

As used herein, the term “inhibiting growth” or “growth inhibition” means a reduction or inhibition in the growth of an insect organism, in some embodiments by at least 10%, 15%, 20%, 25%, 30%, 40%, 45%, 50%, 55%, 60%, 65%, 70%, 75%, 80%, 85%, 90% or 95%. The inhibition of growth of insect can be determined by measuring the weight or size of the insect.

As used herein, the term “mortality” refers to the death of the insects.

As used herein, the term “resistant”, “resistance” and “host plant resistance” refers the ability of a host plant to prevent or reduce infestation and damage of a pest from the group comprising insects, nematodes, pathogens, fungi, viruses, and diseases.

As used herein, the term “insect resistance transgene product”, can mean a “pesticide”, a “Bt” or “Bt polypeptide” where the plant protectant is a protein, or a variant thereof, derived from Bacillus thuringiensis, a “non-Bt” or “non-Bt polypeptide”, where the plant protectant is a protein, or a variant thereof, derived from a bacterium other than Bacillus thuringiensis or a plant, particularly from a fern or other primitive plant, or “RNA” where the plant protectant is an RNA molecule, particularly a hairpin or dsRNA. Transgenic insecticidal products can be expressed from a transgenic event that comprises a transgene encoding the transgenic insect resistance trait.

As used herein, the term “protecting” refers to the avoidance of, or minimizing the amount of attack of plant by a pest to a point where it no longer poses a threat to plant vitality, selective plant death, quality loss and/or reduced yields.

As used herein, the term “crop field” refers to a cultivated expanse of land that a farmer uses to grow a crop species. A crop field ranges in size depending on crop species and purpose. In one example, a crop field can include rows and can be planted at various lengths. In another example, a crop field can be planted by broadcasting the seed throughout the crop field. In a further example, a crop field can be planted by drilling the seed throughout the crop field.

As used herein, the term “modes of action” means the biological or biochemical means by which a pest control strategy or compound inhibits pest feeding and/or increases pest mortality.

As used herein, the term “co-expressing” refers to two or more gene products which are produced at the same time within the same host organism.

As used herein, the term “degenerate” refers to a primer or probe nucleic acid in which certain positions are not defined by a single, specific nucleotide. Thus, in such a degenerate position, the primer or probe sequence can be either one of at least two different nucleotides. Such positions often represent difference in genotypes of the target nucleic acid. A degenerate sequence may also be represented as a mixture of multiple non-degenerate individual sequences which, for the purpose of this disclosure, differ in at least two positions.

As used herein, the term “enzymatically active fragment”, “fragment” or “biologically active portion” include polypeptide fragments comprising amino acid sequences sufficiently identical to a polypeptide and that exhibit insecticidal activity. “Fragments” or “biologically active portions” include polypeptide fragments comprising amino acid sequences sufficiently identical to the amino acid sequence that exhibit insecticidal activity. A biologically active portion of a polypeptide can be a polypeptide that is, for example, 8, 10, 25, 50, 100, 150, 200, 250 or more amino acids in length. Such biologically active portions can be prepared by recombinant techniques and evaluated for insecticidal activity. As used here, a fragment comprises at least 8 contiguous amino acids of a polypeptide. The embodiments encompass other fragments, however, such as any fragment in the protein greater than about 10, 20, 30, 50, 100, 150, 200, 250 or more amino acids.

As used herein, the term “peptide segment” refers to a protein molecule that has been isolated free of other protein sequences and amino acid residues.

As used herein, the term “DNA segment” refers to a DNA molecule that has been isolated free of total genomic DNA of a particular species. Therefore, a DNA segment encoding a protein or peptide refers to a DNA segment that contains protein coding sequences yet is isolated away from, or purified free from, total genomic DNA of the species from which the DNA segment is obtained, which in the instant case is the genome of the Gram-positive bacterial genus, Bacillus, and in particular, the species known as B. thuringiensis. Included within the term “DNA segment”, are DNA segments and smaller fragments of such segments, and also recombinant vectors, including, for example, plasmids, cosmids, phagemids, phage, viruses, and the like.

As used herein, the term “formulated insecticidal protein” refers to a purified or isolated insecticidal protein that has been expressed or placed into a synthetic composition suitable for agricultural application, including but not limited to transgenic plants, sprayable liquid formulations, powdered solid formulations, or granular formulations.

As used herein, the term “expression” refers to the combination of intracellular processes, including transcription and translation undergone by a coding DNA molecule such as a structural gene to produce a polypeptide.

As used herein, the term “transgenic cell” means any cell derived or regenerated from a transformed cell or derived from a transgenic cell. Exemplary transgenic cells include plant calli derived from a transformed plant cell and particular cells such as leaf, root, stem, e.g., somatic cells, or reproductive (germ) cells obtained from a transgenic plant.

As used herein, the term “transgenic plant” means a plant or progeny thereof derived from a transformed plant cell or protoplast, wherein the plant DNA contains an introduced exogenous DNA molecule not originally present in a native, non-transgenic plant of the same strain. The terms “transgenic plant” and “transformed plant” have sometimes been used in the art as synonymous terms to define a plant whose DNA contains an exogenous DNA molecule. However, it is thought more scientifically correct to refer to a regenerated plant or callus obtained from a transformed plant cell or protoplast as being a transgenic plant, and that usage will be followed herein.

As used herein, the term “promoter” refers to a region of DNA that generally is located upstream (towards the 5′ region of a gene) of a gene and is needed to initiate and drive transcription of the gene. A promoter may permit proper activation or repression of a gene that it controls. A promoter may contain specific sequences that are recognized by transcription factors. These factors may bind to a promoter DNA sequence, which results in the recruitment of RNA polymerase, an enzyme that synthesizes RNA from the coding region of the gene. The promoter generally refers to all gene regulatory elements located upstream of the gene, including, upstream promoters, 5′ UTR, introns, and leader sequences.

As used herein, the term “upstream-promoter” refers to a contiguous polynucleotide sequence that is sufficient to direct initiation of transcription. As used herein, an upstream-promoter encompasses the site of initiation of transcription with several sequence motifs, which include TATA Box, initiator sequence, TFIIB recognition elements and other promoter motifs (Jennifer, E. F. et al., (2002) Genes & Dev., 16: 2583-2592). The upstream promoter provides the site of action to RNA polymerase II which is a multi-subunit enzyme with the basal or general transcription factors like, TFIIA, B, D, E, F and H. These factors assemble into a transcription pre-initiation complex that catalyzes the synthesis of RNA from DNA template.

The activation of the upstream-promoter is done by the additional sequence of regulatory DNA sequence elements to which various proteins bind and subsequently interact with the transcription initiation complex to activate gene expression. These gene regulatory elements sequences interact with specific DNA-binding factors. These sequence motifs may sometimes be referred to as cis-elements. Such cis-elements, to which tissue-specific or development-specific transcription factors bind, individually or in combination, may determine the spatiotemporal expression pattern of a promoter at the transcriptional level. These cis-elements vary widely in the type of control they exert on operably linked genes. Some elements act to increase the transcription of operably-linked genes in response to environmental responses (e.g., temperature, moisture, and wounding). Other cis-elements may respond to developmental cues (e.g., germination, seed maturation, and flowering) or to spatial information (e.g., tissue specificity). See, for example, Langridge et al., (1989) Proc. Natl. Acad. Sci. USA 86:3219-23. These cis-elements are located at a varying distance from transcription start point, some cis-elements (called proximal elements) are adjacent to a minimal core promoter region while other elements can be positioned several kilobases upstream or downstream of the promoter (enhancers).

As used herein, the terms “5′ untranslated region” or “5′ UTR” is defined as the untranslated segment in the 5′ terminus of pre-mRNAs or mature mRNAs. For example, on mature mRNAs, a 5′ UTR typically harbors on its 5′ end a 7-methylguanosine cap and is involved in many processes such as splicing, polyadenylation, mRNA export towards the cytoplasm, identification of the 5′ end of the mRNA by the translational machinery, and protection of the mRNAs against degradation.

As used herein, the term “intron” refers to any nucleic acid sequence comprised in a gene (or expressed polynucleotide sequence of interest) that is transcribed but not translated. Introns include untranslated nucleic acid sequence within an expressed sequence of DNA, as well as the corresponding sequence in RNA molecules transcribed therefrom. A construct described herein can also contain sequences that enhance translation and/or mRNA stability such as introns. An example of one such intron is the first intron of gene II of the histone H3 variant of Arabidopsis thaliana or any other commonly known intron sequence. Introns can be used in combination with a promoter sequence to enhance translation and/or mRNA stability.

As used herein, the terms “transcription terminator” or “terminator” is defined as the transcribed segment in the 3′ terminus of pre-mRNAs or mature mRNAs. For example, longer stretches of DNA beyond “polyadenylation signal” site is transcribed as a pre-mRNA. This DNA sequence usually contains transcription termination signal for the proper processing of the pre-mRNA into mature mRNA.

As used herein, the term “3′ untranslated region” or “3′ UTR” is defined as the untranslated segment in a 3′ terminus of the pre-mRNAs or mature mRNAs. For example, on mature mRNAs this region harbors the poly-(A) tail and is known to have many roles in mRNA stability, translation initiation, and mRNA export. In addition, the 3′ UTR is considered to include the polyadenylation signal and transcription terminator.

As used herein, the term “polyadenylation signal” designates a nucleic acid sequence present in mRNA transcripts that allows for transcripts, when in the presence of a poly-(A) polymerase, to be polyadenylated on the polyadenylation site, for example, located 10 to 30 bases downstream of the poly-(A) signal. Many polyadenylation signals are known in the art and are useful for the present disclosure. An exemplary sequence includes AAUAAA and variants thereof, as described in Loke J., et al., (2005) Plant Physiology 138(3); 1457-1468.

As used herein, the term “transformation” encompasses all techniques that a nucleic acid molecule can be introduced into such a cell. Examples include, but are not limited to: transfection with viral vectors; transformation with plasmid vectors; electroporation; lipofection; microinjection (Mueller et al., (1978) Cell 15:579-85); Agrobacterium-mediated transfer; direct DNA uptake; Whiskers™-mediated transformation; and microprojectile bombardment. These techniques may be used for both stable transformation and transient transformation of a plant cell. “Stable transformation” refers to the introduction of a nucleic acid fragment into a genome of a host organism resulting in genetically stable inheritance. Once stably transformed, the nucleic acid fragment is stably integrated in the genome of the host organism and any subsequent generation. Host organisms containing the transformed nucleic acid fragments are referred to as “transgenic” organisms. “Transient transformation” refers to the introduction of a nucleic acid fragment into the nucleus, or DNA-containing organelle, of a host organism resulting in gene expression without genetically stable inheritance.

An exogenous nucleic acid sequence. In one example, a transgene/heterologous coding sequence is a gene sequence (e.g., an herbicide-resistance gene), a gene encoding an industrially or pharmaceutically useful compound, or a gene encoding a desirable agricultural trait. In yet another example, the transgene/heterologous coding sequence is an antisense nucleic acid sequence, wherein expression of the antisense nucleic acid sequence inhibits expression of a target nucleic acid sequence. A transgene/heterologous coding sequence may contain regulatory sequences operably linked to the transgene/heterologous coding sequence (e.g., a promoter). In some embodiments, a polynucleotide sequence of interest is a transgene. However, in other embodiments, a polynucleotide sequence of interest is an endogenous nucleic acid sequence, wherein additional genomic copies of the endogenous nucleic acid sequence are desired, or a nucleic acid sequence that is in the antisense orientation with respect to the sequence of a target nucleic acid molecule in the host organism.

As used herein, the term a transgenic “event” is produced by transformation of plant cells with heterologous DNA, i.e., a nucleic acid construct that includes a transgene/heterologous coding sequence of interest, regeneration of a population of plants resulting from the insertion of the transgene/heterologous coding sequence into the genome of the plant, and selection of a particular plant characterized by insertion into a particular genome location. The term “event” refers to the original transformant and progeny of the transformant that include the heterologous DNA. The term “event” also refers to progeny produced by a sexual outcross between the transformant and another variety that includes the genomic/transgene DNA. Even after repeated back-crossing to a recurrent parent, the inserted transgene/heterologous coding sequence DNA and flanking genomic DNA (genomic/transgene DNA) from the transformed parent is present in the progeny of the cross at the same chromosomal location. The term “event” also refers to DNA from the original transformant and progeny thereof comprising the inserted DNA and flanking genomic sequence immediately adjacent to the inserted DNA that would be expected to be transferred to a progeny that receives inserted DNA including the transgene/heterologous coding sequence of interest as the result of a sexual cross of one parental line that includes the inserted DNA (e.g., the original transformant and progeny resulting from selfing) and a parental line that does not contain the inserted DNA.

As used herein, the terms “Polymerase Chain Reaction” or “PCR” define a procedure or technique in which minute amounts of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51:263 (1987); Erlich, ed., PCR Technology, (Stockton Press, N Y, 1989).

As used herein, the term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is typically a single-stranded sequence, such that amplification efficiency is optimized, but double-stranded sequences can be utilized.

As used herein, the term “probe” refers to an oligonucleotide that hybridizes to a target sequence. In the TaqMan® or TaqMan®-style assay procedure, the probe hybridizes to a portion of the target situated between the annealing site of the two primers. A probe includes about eight nucleotides, about ten nucleotides, about fifteen nucleotides, about twenty nucleotides, about thirty nucleotides, about forty nucleotides, or about fifty nucleotides. In some embodiments, a probe includes from about eight nucleotides to about fifteen nucleotides. A probe can further include a detectable label, e.g., a fluorophore (Texas-Red®, Fluorescein isothiocyanate, etc.,). The detectable label can be covalently attached directly to the probe oligonucleotide, e.g., located at the probe's 5′ end or at the probe's 3′ end. A probe including a fluorophore may also further include a quencher, e.g., Black Hole Quencher™, Iowa Black™, etc.

As used herein, the terms “restriction endonucleases” and “restriction enzymes” refer to bacterial enzymes, each of which cut double-stranded DNA at or near a specific nucleotide sequence. Type-2 restriction enzymes recognize and cleave DNA at the same site, and include but are not limited to XbaI, BamHI, HindIII, EcoRI, XhoI, SalI, KpnI, AvaI, PstI and SmaI.

As used herein, the term “vector” is used interchangeably with the terms “construct”, “cloning vector” and “expression vector” and means the vehicle by which a DNA or RNA sequence (e.g. a foreign gene) can be introduced into a host cell, so as to transform the host and promote expression (e.g. transcription and translation) of the introduced sequence. A “non-viral vector” is intended to mean any vector that does not comprise a virus or retrovirus. In some embodiments a “vector” is a sequence of DNA comprising at least one origin of DNA replication and at least one selectable marker gene. Examples include, but are not limited to, a plasmid, cosmid, bacteriophage, bacterial artificial chromosome (BAC), or virus that carries exogenous DNA into a cell. A vector can also include one or more genes, antisense molecules, and/or selectable marker genes and other genetic elements known in the art. A vector may transduce, transform, or infect a cell, thereby causing the cell to express the nucleic acid molecules and/or proteins encoded by the vector.

The term “plasmid” defines a circular strand of nucleic acid capable of autosomal replication in either a prokaryotic or a eukaryotic host cell. The term includes nucleic acid which may be either DNA or RNA and may be single- or double-stranded. The plasmid of the definition may also include the sequences which correspond to a bacterial origin of replication.

As used herein, the term “selectable marker gene” as used herein defines a gene or other expression cassette which encodes a protein which facilitates identification of cells into which the selectable marker gene is inserted. For example a “selectable marker gene” encompasses reporter genes as well as genes used in plant transformation to, for example, protect plant cells from a selective agent or provide resistance/tolerance to a selective agent. In one embodiment only those cells or plants that receive a functional selectable marker are capable of dividing or growing under conditions having a selective agent. The phrase “marker-positive” refers to plants that have been transformed to include a selectable marker gene.

As used herein, the term “detectable marker” refers to a label capable of detection, such as, for example, a radioisotope, fluorescent compound, bioluminescent compound, a chemiluminescent compound, metal chelator, or enzyme. Examples of detectable markers include, but are not limited to, the following: fluorescent labels (e.g., FITC, rhodamine, lanthanide phosphors), enzymatic labels (e.g., horseradish peroxidase, P-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In an embodiment, a detectable marker can be attached by spacer arms of various lengths to reduce potential steric hindrance.

As used herein, the terms “cassette”, “expression cassette” and “gene expression cassette” refer to a segment of DNA that can be inserted into a nucleic acid or polynucleotide at specific restriction sites or by homologous recombination. As used herein the segment of DNA comprises a polynucleotide that encodes a polypeptide of interest, and the cassette and restriction sites are designed to ensure insertion of the cassette in the proper reading frame for transcription and translation. In an embodiment, an expression cassette can include a polynucleotide that encodes a polypeptide of interest and having elements in addition to the polynucleotide that facilitate transformation of a particular host cell. In an embodiment, a gene expression cassette may also include elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like.

As used herein a “linker” or “spacer” is a bond, molecule or group of molecules that binds two separate entities to one another. Linkers and spacers may provide for optimal spacing of the two entities or may further supply a labile linkage that allows the two entities to be separated from each other. Labile linkages include photocleavable groups, acid-labile moieties, base-labile moieties and enzyme-cleavable groups. The terms “polylinker” or “multiple cloning site” as used herein defines a cluster of three or more Type-2 restriction enzyme sites located within 10 nucleotides of one another on a nucleic acid sequence. In other instances the term “polylinker” as used herein refers to a stretch of nucleotides that are targeted for joining two sequences via any known seamless cloning method (i.e., Gibson Assembly®, NEBuilder HiFiDNA Assembly®, Golden Gate Assembly, BioBrick® Assembly, etc.). Constructs comprising a polylinker are utilized for the insertion and/or excision of nucleic acid sequences such as the coding region of a gene.

As used herein, the term “control” refers to a sample used in an analytical procedure for comparison purposes. A control can be “positive” or “negative”. For example, where the purpose of an analytical procedure is to detect a differentially expressed transcript or polypeptide in cells or tissue, it is generally preferable to include a positive control, such as a sample from a known plant exhibiting the desired expression, and a negative control, such as a sample from a known plant lacking the desired expression.

As used herein, the term “plant” includes a whole plant and any descendant, cell, tissue, or part of a plant. A class of plant that can be used in the present disclosure is generally as broad as the class of higher and lower plants amenable to mutagenesis including angiosperms (monocotyledonous and dicotyledonous plants), gymnosperms, ferns and multicellular algae. Thus, “plant” includes dicot and monocot plants. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf, stem; fruit; seed; and root.

A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete-producing cell, or a cell or collection of cells that can regenerate into a whole plant. As such, a seed, which comprises multiple plant cells and is capable of regenerating into a whole plant, is considered a “plant cell” in embodiments herein.

As used herein, the term “small RNA” or “RNAi-mediating molecule” refers to several classes of non-protein-coding ribonucleic acid (ncRNA). The term small RNA or “RNAi-mediating molecule” describes the short chains of ncRNA produced in bacterial cells, animals, plants, and fungi. These short chains of ncRNA may be produced naturally within the cell or may be produced by the introduction of an exogenous sequence that expresses the short chain or ncRNA. The small RNA or “RNAi-mediating molecule” sequences do not directly code for a protein, and differ in function from other RNA in that small RNA or “RNAi-mediating molecule” sequences are only transcribed and not translated. The small RNA or “RNAi-mediating molecule” sequences are involved in other cellular functions, including gene expression and modification. Small RNA or “RNAi-mediating” molecules are usually made up of about 20 to 30 nucleotides. The small RNA sequences or “RNAi-mediating molecule” may be derived from longer precursors. The precursors form structures that fold back on each other in self-complementary regions; they are then processed by the nuclease DICER in animals or DCL1 in plants.

Many types of small RNA or “RNAi-mediating molecule” exist either naturally or produced artificially, including but not limited to microRNAs (miRNAs), small interfering RNAs (siRNAs), antisense RNA, short/small hairpin RNA (shRNA), and small nucleolar RNAs (snoRNAs). Certain types of small RNA or “RNAi-mediating molecule”, such as microRNA and siRNA, are important in gene silencing and RNA interference (RNAi). Gene silencing is a process of genetic regulation in which a gene that would normally be expressed is “turned off” by an intracellular element, in this case, the small RNA or “RNAi-mediating molecule”. The protein that would normally be formed by this genetic information is not formed due to interference, and the information coded in the gene is blocked from expression.

As used herein, the term “small RNA” or “RNAi-mediating molecule” encompasses RNA molecules described in the literature as “tiny RNA” (Storz, (2002) Science 296:1260-3; Illangasekare et al., (1999) RNA 5:1482-1489); prokaryotic “small RNA” (sRNA) (Wassarman et al., (1999) Trends Microbiol. 7:37-45); eukaryotic “noncoding RNA (ncRNA)”; “micro-RNA (miRNA)”; “small non-mRNA (snmRNA)”; “functional RNA (fRNA)”; “transfer RNA (tRNA)”; “catalytic RNA” [e.g., ribozymes, including self-acylating ribozymes (Illangaskare et al., (1999) RNA 5:1482-1489); “small nucleolar RNAs (snoRNAs),” “tmRNA” (a.k.a. “10S RNA,” Muto et al., (1998) Trends Biochem Sci. 23:25-29; and Gillet et al., (2001) Mol Microbiol. 42:879-885); endo-siRNA (Ghildiyal, M., et al., Endogenous siRNAs derived from transposons and mRNAs in Drosophila somatic cells. Science, 2008. 320(5879): p. 1077-81.); piRNA (Aravin, A., et al., A novel class of small RNAs bind to MILI protein in mouse testes. Nature, 2006. 442(7099): p. 203-7.); RNAi-mediating molecules including without limitation “small interfering RNA (siRNA),” “endoribonuclease-prepared siRNA (e-siRNA),” “short/small hairpin RNA (shRNA),” and “small temporally regulated RNA (stRNA),” “diced siRNA (d-siRNA),” and aptamers, oligonucleotides and other synthetic nucleic acids that comprise at least one uracil base.

As used herein, the term DICER recognition sequence is any stretch of polynucleotides that are recognized and bound by the DICER enzyme for subsequent cleavage. The double-stranded molecule generated by DICER activity upon a shRNA molecule may be separated into two single-stranded shRNAs; the “STAR/passenger strand” and the “guide strand.” The STAR/passenger strand may be degraded, and the guide strand may be incorporated into the RISC complex. Post-transcriptional inhibition occurs by specific hybridization of the guide strand with a specifically complementary polynucleotide of an mRNA molecule, and subsequent cleavage by the enzyme, Argonaute (e.g., a catalytic component of the RISC complex).

As used herein, the term DROSHA recognition sequence, is any stretch of polynucleotides that are recognized and bound by the DROSHA enzyme for subsequent cleavage.

Unless otherwise specifically explained, all technical and scientific terms used herein have the same meaning as commonly understood by those of ordinary skill in the art to which this disclosure belongs. Definitions of common terms in molecular biology can be found in, for example: Lewin, Genes V, Oxford University Press, 1994 (ISBN 0-19-854287-9); Kendrew et al. (eds.), The Encyclopedia of Molecular Biology, Blackwell Science Ltd., 1994 (ISBN 0-632-02182-9); and Meyers (ed.), Molecular Biology and Biotechnology: A Comprehensive Desk Reference, VCH Publishers, Inc., 1995 (ISBN 1-56081-569-8).

### EMBODIMENTS

In an embodiment, the subject disclosure relates to methods and compositions to produce an RNA complex. The complex comprises a cell-penetrating peptide and an RNA molecule. In some embodiments, a cell-penetrating peptide is a peptide that enhances penetration of a polypeptide containing the peptide into a cell compared to a polypeptide lacking the cell-penetrating peptide. Penetration of a polypeptide into a cell can be detected using any method known in the art or described herein, e.g., Western blot, immunohistochemistry, immunofluorescence, and other similar assays performed, e.g., on a fixed cell or a cell lysate. In some embodiments, the cell-penetrating peptide comprises or consists of a sequence of SEQ ID NO:1 (BAPC1) complexed with SEQ ID NO:66 (BAPC1), SEQ ID NO:2 (Knotted-1), SEQ ID NO:3 (7-zein), SEQ ID NO:4 (CyLoP-1) or SEQ ID NO:5 (MPG) or a fragment or variant thereof that is capable of enhancing penetration of the polypeptide (e.g., compared to a polypeptide not containing the cell-penetrating peptide). In some embodiments, the cell-penetrating peptide comprises or consist of a sequence of SEQ ID NO:6 (TAT), SEQ ID NO:7 (TAT2) and SEQ ID NO:8 (M-TAT) as described in Pooga et al., (2001) “Cellular translocation of proteins by transportan” FASEB J 15, 1451-1453; SEQ ID NO: 9 (PepR) and SEQ ID NO:10 (PepM) as described in Freire et al., (2014), Nucleic acid delivery by cell-penetrating peptides derived from dengue virus capsid protein: design and mechanism of action. Volume 281, Issue 1, Pages 191-215 (herein incorporated by reference). In some embodiments, the cell-penetrating peptide comprises of a sequence of SEQ ID NO:1-SEQ ID NO:66. In other embodiments, the cell-penetrating peptide consists of a sequence of SEQ ID NO:1-SEQ ID NO:66. In further embodiments, the cell-penetrating peptide consists essentially of a sequence of SEQ ID NO:1-SEQ ID NO:66. In some embodiments, the fragment has one, two, or three amino acid deletions from the N- and/or C-terminus of an amino acid sequence provided herein. In some embodiments, the variant has one, two, or three amino acid substitutions (e.g., conservative amino acid substitutions) in an amino acid sequence provided herein.

TABLE 1
Selected CPPs

| Name | Sequence (N to C) | Type/Description |
|---|---|---|
| BAPC1 (e.g., | bis(FLIVI)-K-KKKK (SEQ ID | One of two branched |
| BAP tofect) | NO: 1) | CPPs that complexes |
| | | with the other BAPC1 |
| | | (i.e., SEQ ID NO: 66) |
| BAPC1 (e.g., | bis(FLIVIGSII)-K-KKKK (SEQ ID | One of two branched |
| BAP tofect) | NO: 66) | CPPs that complexes |
| | | with the other BAPC1 |
| | | (i.e., SEQ ID NO: 1) |
| CyLoP-1 | CRWRWKCCKK (SEQ ID NO: 4) | no fluorescent, |
| | | amphipathic |
| FAM_CyLoP- | CRWRWKCCKK (SEQ ID NO: 4) | FAM at N-Terminus |
| 1 | | |
| MPG | GALFLGFLGAAGSTMGAWSQP | no fluorescent |
| | KKKRKV (SEQ ID NO: 5) | |
| MPG_FAM | GALFLGFLGAAGSTMGAWSQP | FAM at C-Terminus |
| | KKKRKV (SEQ ID NO: 5) | |
| Knotted-1 | KQINNWFINQRKRHWK (SEQ | no fluorescent |
| | ID NO: 2) | |
| CY3_Knotted- | KQINNWFINQRKRHWK (SEQ | CY3 at N-Terminus |
| 1 | ID NO: 2) | |
| FAM_Knotted- | KQINNWFINQRKRHWK (SEQ | FAM at N-Terminus |
| 1 | ID NO: 2) | |
| γ-Zein | VRLPPPVRLPPPL VRPPPL (SEQ | no fluorescent |
| | ID NO: 3) | |
| TAT | RKKRRQRRR-amide (SEQ ID | Linear |
| | NO: 6) | |
| TAT2 | RKKRRQRRRRKKRRQRRR- | Linear |
| | amide (SEQ ID NO: 7) | |
| M-TAT | AKKRRQRRR-amide (SEQ ID | Linear |
| | NO: 8) | |
| PepR | LKRWGTIKKSKAINVLRGFRKE | Linear |
| | IGRMLNILNRRRR (SEQ ID | |
| | NO: 9) | |
| PepM | KLFMALVAFLRFLTIPPTAGILK | Linear |
| | RWGTI (SEQ ID NO: 10) | |
| R9 | RRRRRRRRR (SEQ ID NO: 11) | Cationic |
| D-R9 | rrrrrrrrr (D form) (SEQ ID NO: 12) | Cationic |
| R-12 | RRRRRRRRRRRR (SEQ ID | Cationic |
| | NO: 13) | |
| R9-TAT | GRRRRRRRRRPPQ (SEQ ID | Cationic |
| | NO: 14) | |
| Tat (49-57) | RKKRRQRRR (SEQ ID NO: 15) | Cationic |
| Tat2 | RKKRRQRRRRKKRRQRRR | Cationic |
| | (SEQ ID NO: 16) | |

TABLE 2
Alternative CPPs

| Name | Sequence (N to C) | Type/description |
|---|---|---|
| BP100 | KKLFKKILKYL (SEQ ID | Amphipathic |
| | NO: 17) | |
| 2BP100 | KKLFKKILKYLKKLFKKILKY | Cationic |
| | L (SEQ ID NO: 18) | |
| Rev(34-50) | TRQARRNRRRRWRERQR | Cationic |
| | (SEQ ID NO: 19) | |
| KH9 | KHKHKHKHKHKHKHKHKH | Cationic |
| | (SEQ ID NO: 20) | |
| K9 | KKKKKKKKK (SEQ ID NO: 21) | Cationic |
| K18 | KKKKKKKKKKKKKKKKKK | Cationic |
| | (SEQ ID NO: 22) | |
| Pen2W2F | RQIKIFFQNRRMKFKK (SEQ | Cationic |
| | ID NO: 23) | |
| DPV3 | RKKRRRESRKKRRRES (SEQ | Cationic |
| | ID NO: 24) | |
| 6-Oct | GRKRKKRT (SEQ ID NO: 25) | Cationic |
| Retro-Tat | RRRQRRKKR (SEQ ID NO: 26) | Cationic |
| (57-49) | | |
| Sc18 | GLRKRLRKFRNKIKEK (SEQ | Amphipathic |
| | ID NO: 27) | |
| KLA10 | KALKKLLAKWLAAAKALL | Amphipathic |
| | (SEQ ID NO: 28) | |
| IX | QLALQLALQALQAALQLA | Hydrophobic |
| | (SEQ ID NO: 29) | |
| XI | LKTLATALTKLAKTLTTL | Hydrophobic |
| | (SEQ ID NO: 30) | |
| No. 14-12 | RAWMRWYSPTTRRYG (SEQ | Amphipathic |
| | ID NO: 31) | |
| pVEC | LLIILRRRIRKQAHAHSK (SEQ | Amphipathic |
| | ID NO: 32) | |
| PenArg | RQIRIWFQNRRMRWRR (SEQ | Amphipathic |
| | ID NO: 33) | |
| M918 | MVTVLFRRLRIRRACGPPRVR | Amphipathic |
| | V (SEQ ID NO: 34) | |
| Penetratin | RQIKIWFQNRRMKWKK (SEQ | Amphipathic |
| | ID NO: 35) | |
| PolyP 3 | VRLPPPVRLPPPVRLPPP (SEQ | Hydrophobic |
| (SAP) | ID NO: 36) | |
| dhvar5 | LLLFLLKKRKKRKY (SEQ ID | Amphipathic |
| | NO: 37) | |
| HPV33L2- | SYFILRRRRKRFPYFFTDVRV | Amphipathic |
| 445/467 | AA (SEQ ID NO: 38) | |
| buforin II | RAGLQFPVGR VHRLLRK | Amphipathic |
| (5-21) | (SEQ ID NO: 39) | |
| scrambled | IAARIKLRSRQHIKLRHL (SEQ | Amphipathic |
| pVEC | ID NO: 40) | |
| HPV33L2- | SYDDLRRRRKRFPYFFTDVRV | Amphipathic |
| DD447 | AA (SEQ ID NO: 41) | |
| LAH4 | KKALLALALHHLAHLALHLA | Hydrophobic |
| | LALKKA (SEQ ID NO: 42) | |
| ppTG1 | GLFKALLKLLKSLWKLLLKA | Amphipathic |
| | (SEQ ID NO: 43) | |
| Transportan | GWTLNSAGYLLGKINLKALA | Hydrophobic |
| (TP) | ALAKKIL (SEQ ID NO: 44) | |
| 2× ppTG1 | GLFKALLKLLKSLWKLLLKA | Amphipathic |
| | GLFKALLKLLKSLWKLLLKA | |
| | (SEQ ID NO: 45) | |
| pAntpHD | RQIKIWFPNRRMKWKK (SEQ | Amphipathic |
| (Pro50) | ID NO: 46) | |
| pAntp | QIKIWFQNRRMKWKK (SEQ | Amphipathic |
| (44-58) | ID NO: 47) | |
| Crot(27-39) | KMDCRWRWKCCKK(SEQ ID | Amphipathic |
| | NO: 48) | |
| Crot(27-39) | MDCRWRWKCCKK (SEQ ID | Amphipathic |
| derevative | NO: 49) | |
| (1) | | |
| Crot(27-39) | KCGCRWRWKCGCKK (SEQ | Amphipathic |
| derevative | ID NO: 50) | |
| (2) | | |
| CyLoP-1 | CRWRWKCCKK (SEQ ID | Amphipathic |
| | NO: 51) | |
| Inv3 | TKRRITPKDVIDVRSVTTEINT | Hydrophobic |
| | (SEQ ID NO: 52) | |
| Inv5 | AEKVDPVKLNLTLSAAAEAL | Hydrophobic |
| | TGLGDK (SEQ ID NO: 53) | |
| Inv3.5 | TKRRITPKDVIDVRSVTTKINT | Hydrophobic |
| | (SEQ ID NO: 54) | |
| ARF(1-22) | MVRRFLVTLRIRRACGPPRVR | Amphipathic |
| | V (SEQ ID NO: 55) | |
| Cyt C 77- | GTKMIFVGIKKKEERADLIAY | Amphipathic |
| 101 | LKKA (SEQ ID NO: 56) | |
| hLF peptide | KCFQWQRNMRKVRGPPVSCI | Amphipathic |
| | KR (SEQ ID NO: 57) | |
| Glu-Oct-6 | EEEAAGRKRKKRT (SEQ ID | Amphipathic |
| | NO: 58) | |
| M511 | FLGKKFKKYFLQLLK (SEQ ID | Amphipathic |
| | NO: 59) | |
| G53-4 | FLIFIRVICIVIAKLKANLMCK | Hydrophobic |
| | T (SEQ ID NO: 60) | |
| M591 | YIVLRRRRKRVNTKRS (SEQ | Amphipathic |
| | ID NO: 61) | |
| E162 | KTVLLRKLLKLLVRKI (SEQ | Amphipathic |
| | ID NO: 62) | |
| E165 | LLKKRKVVRLIKFLLK (SEQ | Amphipathic |
| | ID NO: 63) | |
| M867 | KKICTRKPRFMSAWAQ (SEQ | Amphipathic |
| | ID NO: 64) | |
| MG2d | GIGKFLHSAKKWGKAFVGQI | Hydrophobic |
| | MNC (SEQ ID NO: 65) | |

In some embodiments the cell-penetrating peptide is complexed to an RNA molecule. In various aspects the RNA molecule may be an mRNA molecule. As used herein, the term “messenger RNA” (mRNA) refers to any polynucleotide which encodes at least one peptide or polypeptide of interest and which is capable of being translated to produce the encoded peptide polypeptide of interest in vitro, in vivo, in situ or ex vivo. An mRNA has been transcribed from a DNA sequence by an RNA polymerase enzyme, and interacts with a ribosome synthesize genetic information encoded by DNA. Generally, mRNA are classified into two sub-classes: pre-mRNA and mature mRNA. Precursor mRNA (pre-mRNA) is mRNA that has been transcribed by RNA polymerase but has not undergone any post-transcriptional processing (e.g., 5′ capping, splicing, editing, and polyadenylation). Mature mRNA has been modified via post-transcriptional processing (e.g., spliced to remove introns and polyadenylated) and is capable of interacting with ribosomes to perform protein synthesis. mRNA can be isolated from tissues or cells by a variety of methods. For example, a total RNA extraction can be performed on cells or a cell lysate and the resulting extracted total RNA can be purified (e.g., on a column comprising oligo-dT beads) to obtain extracted mRNA.

Alternatively, mRNA can be synthesized in a cell-free environment, for example by in vitro transcription (IVT). An “in vitro transcription template” as used herein, refers to deoxyribonucleic acid (DNA) suitable for use in an IVT reaction for the production of messenger RNA (mRNA). In some embodiments, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template.

The mRNA may have a nucleotide sequence of a native or naturally occurring mRNA or encoding a native or naturally occurring peptide. Alternatively the mRNA may have a nucleotide sequence having a percent identity to the nucleotide sequence of a native or naturally occurring mRNA or mRNA may have a nucleotide sequence encoding a peptide having a percent identity to the nucleotide sequence of a native or naturally occurring peptide. In some embodiments, the mRNA has a length of or greater than about 0.5 kb, 1 kb, 1.5 kb, 2 kb, 2.5 kb, 3 kb, 3.5 kb, 4 kb, 4.5 kb, or 5 kb.

In some embodiments, the agronomic trait encoded by the mRNA is a cystosolic protein. In some embodiments, the agronomic trait encoded by the mRNA is a secreted protein. In some embodiments, the agronomic trait encoded by the mRNA is an enzyme. In some embodiments, the enzyme is a lysosomal enzyme.

In other aspects the RNA molecule may be an RNAi-mediating (e.g., small RNA) molecule. RNA interference (RNAi) is a sequence-specific RNA degradation process that provides a relatively easy and direct way to knockdown, or silence, theoretically any gene containing a complementary sequence. In naturally-occurring RNAi, a double-stranded RNA (dsRNA) is cleaved by an RNase III/helicase protein, Dicer, into small interfering RNA (siRNA) molecules, dsRNA of 19-27 nucleotides (nt) with 2-nt overhangs at the 3′ ends. Afterwards, the siRNAs are incorporated into a multicomponent-ribonuclease called RNA-induced-silencing-complex (RISC). One strand of siRNA remains associated with RISC to guide the complex towards a cognate RNA that has a sequence complementary to the guider ss-siRNA in RISC. This siRNA-directed endonuclease digests the RNA, resulting in truncation and inactivation of the targeted RNA. Recent studies have revealed the utility of chemically synthesized 21-27-nt siRNAs that exhibit RNAi effects in mammalian cells and have demonstrated that the thermodynamic stability of siRNA hybridization (at terminals or in the middle) plays a central role in determining the molecule's function. More detailed characteristics of RISC, siRNA molecules, and RNAi have been described in the scientific literature.

The utility of RNAi in downregulation of insect cell gene expression has been shown successfully in the laboratory by utilizing either chemically synthesized siRNAs or endogenously expressed siRNA. The endogenous siRNA is first expressed as hairpin RNAs (hRNAs) by an expression vector (plasmid or virus vector), and then processed by Dicer to become functional siRNAs.

Importantly, it is presently not possible to predict with any degree of confidence which of many possible candidate siRNA sequences potentially targeting a transcriptomic sequence (e.g., oligonucleotides of about 16-30 base pairs) will in fact exhibit effective siRNA activity. Instead, individual, specific candidate siRNA polynucleotide or oligonucleotide sequences are naturally generated and tested.

In some aspects RNAi refers to the biological process of inhibiting, decreasing, or downregulating gene expression in a cell, and which is mediated by RNAi mediating or small RNA molecules (e.g., siRNAs, miRNAs, shRNAs, and dsRNAs), see for example Zamore and Haley, 2005, Science 309:1519-1524; Vaughn and Martienssen, 2005, Science 309:1525-1526; Zamore et al., 2000, Cell 101:25-33; Bass, 2001, Nature 411:428-429; Elbashir et al., 2001, Nature 411:494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science 297:1818-1819; Volpe et al., 2002, Science 297:1833-1837; Jenuwein, 2002, Science 297:2215-2218; and Hall et al., 2002, Science 297:2232-2237; Hutvagner and Zamore, 2002, Science 297:2056-60; McManus et al., 2002, RNA 8:842-850; Reinhart et al., 2002, Gene & Dev. 16:1616-1626; and Reinhart & Bartel, 2002, Science 297:1831). Additionally, the term “RNA interference” (or “RNAi”) is meant to be equivalent to other terms used to describe sequence-specific RNA interference, such as post-transcriptional gene silencing, translational inhibition, transcriptional inhibition, or epigenetics. For example, single-stranded RNA molecules of the invention can be used to epigenetically silence genes at either the post-transcriptional level or the pre-transcriptional level. In a non-limiting example, epigenetic modulation of gene expression by single-stranded RNA molecules of the invention can result from modification of chromatin structure or methylation patterns to alter gene expression (see, for example, Verdel et al., 2004, Science 303:672-676; Pal-Bhadra et al., 2004, Science 303:669-672; Allshire, 2002, Science 297:1818-1819; Volpe et al., 2002, Science 297:1833-1837; Jenuwein, 2002, Science 297:2215-2218; and Hall et al., 2002, Science 297:2232-2237). In another non-limiting example, modulation of gene expression by single-stranded RNA molecules of the invention can result from cleavage of RNA (either coding or non-coding RNA) via RISC, or via translational inhibition, as is known in the art or modulation can result from transcriptional inhibition (see for example Janowski et al., 2005, Nature Chemical Biology 1:216-222).

The terms “inhibit,” “downregulate,” “reduce” or “knockdown” as used herein refer to their meanings as are generally accepted in the art. With reference to exemplary RNAi-mediating molecules of the invention, the terms generally refer to the reduction in the (i) expression of a gene or target sequence and/or the level of RNA molecules encoding one or more proteins or protein subunits, and/or (ii) the activity of one or more proteins or protein subunits, below that observed in the absence of the RNAi-mediating molecules of the invention. Downregulation can also be associated with post-transcriptional silencing, such as RNAi-mediated cleavage, or by alteration in DNA methylation patterns or DNA chromatin structure. Inhibition, downregulation, reduction or knockdown with an RNAi agent can be in reference to an inactive molecule, an attenuated molecule, an RNAi agent with a scrambled sequence, or an RNAi agent with mismatches. The phrase “gene silencing” refers to a partial or complete loss-of-function through targeted inhibition of an endogenous target gene in a cell. As such, the term is used interchangeably with RNAi, “knockdown,” “inhibition,” “downregulation,” or “reduction” of expression of a target gene.

To determine the extent of inhibition, a test sample (e.g., a biological sample from an organism of interest expressing the target gene(s) or target sequence(s) or a sample of cells in culture expressing the target gene/sequence) can be contacted with an RNAi-mediating molecule that silences, reduces, or inhibits expression of a target gene or sequence. Expression of the target gene/sequence in the test sample is compared to expression of the target gene/sequence in a control sample (e.g., a biological sample from an organism of interest expressing the target gene/sequence or a sample of cells in culture expressing the target gene/sequence) that is not contacted with the RNAi-mediating molecule. Control samples (i.e., samples expressing the target gene/sequence) are assigned a value of 100%. Silencing, inhibition, or reduction of expression of a target gene/sequence is achieved when the value of the test sample relative to the control sample is about 95%, 90%, 85%, 80%, 75%, 70%, 65%, 60%, 55%, 50%, 45%, 40%, 35%, 30%, 25%, 20%, or 10%. Suitable assays include, e.g., examination of protein or mRNA levels using techniques known to those of skill in the art, such as dot blots, Northern blots, in situ hybridization, ELISA, microarray hybridization, immunoprecipitation, enzyme function, as well as phenotypic assays known to those of skill in the art.

In some embodiments, the RNAi-mediating molecule comprises a first strand and a second strand, wherein a) the first strand and the second form a duplex; b) the first strand comprises a guide region of at least 11 bases, wherein the guide region comprises a seed region comprising bases 1-N of the guide strand, wherein N=7 or N=8; and c) the second strand comprises a non-guide region of at least 11 bases, wherein the non-guide region comprises a bulge sequence opposite of any one or more of bases 1−(N+2) of the guide region in the duplex. In some embodiments, wherein N=7 and the bulge is opposite base 1, 2, 3, 4, 5, 6, 7, 8, or 9 of the guide region. In other embodiments, N=8 and the bulge is opposite base 1, 2, 3, 4, 5, 6, 7, 8, 9, or 10 of the guide region.

In some embodiments, the RNAi-mediating molecule comprises a first strand and a second strand, wherein a) the first strand and the second form a duplex, b) the first strand comprises a guide region of at least 9 bases, wherein the guide region comprises a seed region comprising bases 2-7 or 2-8 of the guide strand, and c) the second strand comprises a non-guide region of at least 9 bases, wherein the non-guide region comprises a bulge sequence opposite of base 1 or base 9 of the guide region in the duplex.

In some embodiments, the first strand and the second strand are linked by means of an RNA (e.g., an RNA linker) capable of forming a loop structure. As is commonly known in the art, an RNA loop structure (e.g., a stem-loop or hairpin) is formed when an RNA molecule comprises two sequences of RNA that base pair together separated by a sequence of RNA that does not base pair together. For example, a loop structure may form in the RNA molecule A-B-C if sequences A and C are complementary or partially complementary such that they base pair together, but the bases in sequence B do not base pair together.

In some embodiments, the RNA capable of forming a loop structure comprises from 4 to 50 nucleotides. In certain embodiments, the RNA capable of forming a loop structure comprises 13 nucleotides. In some embodiments, the number of nucleotides in the RNA capable of forming a loop is from 4 to 50 nucleotides or any integer therebetween. In some embodiments, from 0-50% of the loop can be complementary to another portion of the loop. As used herein, the term “loop structure” is a sequence that joins two complementary strands of nucleic acid. In some embodiments, 1-3 nucleotides of the loop structure are contiguous to the complementary strands of nucleic acid and may be complementary to 1-3 nucleotides of the distal portion of the loop structure. For example, the three nucleotides at the 5′ end of the loop structure may be complementary to the three nucleotides at the 3′ end of the loop structure.

In additional aspects the RNAi-mediating molecule may be a dsRNA molecule. In some aspects the dsRNA contains a sequence which corresponds to the target region of the target gene. It is not absolutely essential for the whole of the dsRNA to correspond to the sequence of the target region. For example, the dsRNA may contain short non-target regions flanking the target-specific sequence, provided that such sequences do not affect performance of the dsRNA in RNA inhibition to a material extent.

In further aspects, the dsRNA may contain one or more substitute bases in order to optimize performance in RNAi. It will be apparent to the skilled reader how to vary each of the bases of the dsRNA in turn and test the activity of the resulting dsRNAs (e.g. in a suitable in vitro test system) in order to optimize the performance of a given dsRNA. In some aspects the dsRNA may further contain DNA bases, non-natural bases or non-natural backbone linkages or modifications of the sugar-phosphate backbone, for example to enhance stability during storage or enhance resistance to degradation by nucleases.

It has been previously reported that the formation of short interfering RNAs (siRNAs) of about 21 bp is desirable for effective gene silencing. Therefore, in one embodiment, the double-stranded RNA fragment (or region) will itself preferably be at least 17 bp in length, preferably 18 or 19 bp in length, more preferably at least 20 bp, more preferably at least 21 bp, or at least 22 bp, or at least 23 bp, or at least 24 bp, 25 bp, 26 bp or at least 27 bp in length. The expressions “double-stranded RNA fragment” or “double-stranded RNA region” refer to a small entity of the double-stranded RNA corresponding with (part of) the target gene.

Generally, the double-stranded RNA is preferably between about 17-1500 bp, even more preferably between about 80-1000 bp and most preferably between about 17-27 bp or between about 80-250 bp; such as double-stranded RNA regions of about 17 bp, 18 bp, 19 bp, 20 bp, 21 bp, 22 bp, 23 bp, 24 bp, 25 bp, 27 bp, 50 bp, 80 bp, 100 bp, 150 bp, 200 bp, 250 bp, 300 bp, 350 bp, 400 bp, 450 bp, 500 bp, 550 bp, 600 bp, 650 bp, 700 bp, 900 bp, 100 bp, 1100 bp, 1200 bp, 1300 bp, 1400 bp or 1500 bp.

The upper limit on the length of the double-stranded RNA may be dependent on i) the requirement for the dsRNA to be taken up by the insect and ii) the requirement for the dsRNA to be processed within the cell into fragments that direct RNAi. The chosen length may also be influenced by the method of synthesis of the RNA and the mode of delivery of the RNA to the cell. Preferably the double-stranded RNA to be used in the methods of the invention will be less than 10,000 bp in length, more preferably 1000 bp or less, more preferably 500 bp or less, more preferably 300 bp or less, more preferably 100 bp or less. For any given target gene and insect, the optimum length of the dsRNA for effective inhibition may be determined by experiment.

The double-stranded RNA may be fully or partially double-stranded. Partially double-stranded RNAs may include short single-stranded overhangs at one or both ends of the double-stranded portion, provided that the RNA is still capable of being taken up by insects and directing RNAi. The double-stranded RNA may also contain internal non-complementary regions.

In some aspects the RNA molecule may be an siRNA molecule. In embodiments “siRNA” (also “short interfering RNA” or “small interfering RNA”) is given its ordinary meaning accepted in the art, generally referring to a duplex (sense and antisense strands) of complementary RNA oligonucleotides which may or may not comprise 3′ overhangs of about 1 to about 4 nucleotides and which mediate RNA interference.

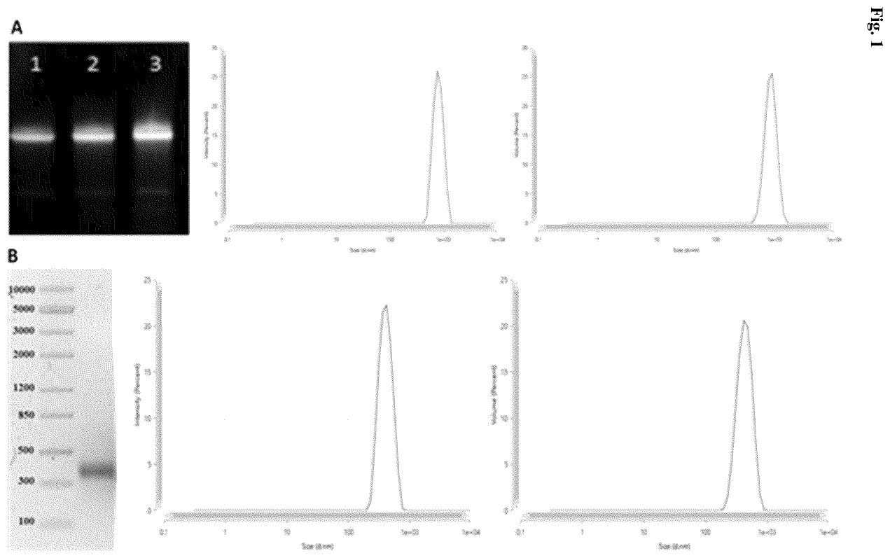

In further aspects the RNA molecule may be a micro-RNA. MicroRNAs (miRNAs) are non-protein coding RNAs, generally between about 19 to about 25 nucleotides in length, that guide interference in trans of target RNA transcripts, negatively regulating the expression of genes (Ambros (2001) Cell 107 (7):823-6; Bartel (2004) Cell 116 (2):281-97). Numerous miRNA genes have been identified and are made publicly available in several databases (e.g. “miRBase” Griffiths-Jones et al. (2003) Nucleic Acids Res., 31:439-441). MiRNAs were first reported in nematodes and have since been identified in other invertebrates; see, for example, Lee and Ambros (2001) Science, 294:862-864; Lim et al. (2003) Genes Dev., 17:991-1008; Stark et al. (2007) Genome Res., 17:1865-1879. Transcription of miRNA genes may be under the control of an miRNA gene's own promoter. However, the biogenesis pathways of microRNAs can vary depending on their genomic origins, for example up to a third of animal miRNAs are thought to be derived from introns (mirtrons) (Okamura et al. (2008) Cell Cycle 7 (18):2840-5; Westholm and Lai (2011) Biochimie 93 (11):1897-904). MiRNA genes may be isolated or appear in clusters in the genome; they can also be located entirely or partially within introns of both protein-coding and non-protein-coding, see Kim (2005) Nature Rev. Mol Cell Biol., 6:376-385; (Westholm and Lai (2011) Biochimie 93 (11):1897-904). The primary transcript (pri-miRNA) can be quite long (several kilobases) and can be monocistronic or polycistronic, containing one or more precursor miRNAs (pre-miRNAs) (fold-back structures containing a stem-loop arrangement that is processed to the mature miRNA), as well as the usual 5′ cap and polyadenylated tail of an mRNA. See, for example, FIG. 1 in Kim (2005) Nature Rev. Mol. Cell Biol., 6:376-385.

A single mature miRNA is precisely processed from a given precursor, and therefore such “artificial” miRNAs (engineered miRNA, short/small hairpin RNAs (shRNA), shRNAmir, shRNA-miR, shmiRs, etc.) offer an advantage over double-stranded RNA (dsRNA) in that only a specific miRNA sequence is expressed, limiting potential off-target effects. Although animal miRNAs typically interact with imperfect target sequences in the 3′ UTR, artificial miRNAs with perfect target complementarity will guide target cleavage (see Zeng et al. (2003) RNA, 9:112-123 and Zeng et al (2003) Proc. Natl. Acad. Sci. U.S.A., 100:9779-9784).

In other embodiment, the subject disclosure relates to the linkage of the cell-penetrating peptide to the RNA molecule. In some aspects more than one type of cell-penetrating peptide can be linked to a RNA molecule. The ratio (molar ratio) of cell-penetrating peptide to a RNA molecule that can be used when crosslinking can be at least about 1:1, 2:1, 3:1, 4:1, 5:1, 6:1, 7:1, 8:1, 9:1, 10:1 15:1, 20:1, 30:1, 40:1, or 50:1, 100:1, 200:1, 300:1, 400:1, 500:1, 600:1, 700:1, 800:1, 900:1 or 1000:1 for example. The ratio (molar ratio) of RNA molecule to a cell-penetrating peptide that can be used when crosslinking can be at least about 1:1, 2:1, 3:1, 4:1, 5:1, 6:1, 7:1, 8:1, 9:1, 10:1 15:1, 20:1, 30:1, 40:1, or 50:1, 100:1, 200:1, 300:1, 400:1, 500:1, 600:1, 700:1, 800:1, 900:1 or 1000:1 for example. In other aspects, the average number of cell-penetrating peptide crosslinked to a RNA molecule may be at least 1, 2, 3, 4, 5, 6, 7, 8, 9, 10, 11, 12, 13, 14, 15, 16, 17, 18, 19, 20, 21, 22, 23, 24, or 25, or at least 5-10, 5-15, 5-20, or 5-25.

In a further embodiment, the cell-penetrating peptide and the RNA molecule may be linked to one another through non-covalent linkage. The terms “non-covalently linked,” “non-covalently attached,” “non-covalently associated,” “non-covalent linkage,” “non-covalent interaction” and the like are used interchangeably herein. A non-covalent linkage herein refers to an interaction between atoms in which electrons are not shared. This type of interaction is weaker than a covalent linkage. Hydrophobic interactions represent an example of a non-covalent linkage that may occur between an RNA molecule and one or more CPPs. Other examples of non-covalent linkages that may apply herein include electrostatic forces (e.g., ionic, hydrogen bonding) and Van der Waals forces (London dispersion forces).

In other embodiments, a direct linkage via an amide bridge can be utilized to link the RNA molecule and one or more CPPs. Such a linkage is applicable to linking a first CPP to one or more CPPs wherein the components to be linked have reactive amino or carboxy groups. More specifically, if the components to be linked are peptides, polypeptides or proteins, a peptide bond is preferred. Such a peptide bond may be formed using a chemical synthesis involving both components (an N-terminal end of one component and the C-terminal end of the other component) to be linked, or may be formed directly via a protein synthesis of the entire peptide sequence of both components, wherein both (protein or peptide) components are preferably synthesized in one step.

In other embodiments, the cell-penetrating peptide can be linked via the N-terminal end to an RNA molecule. In further embodiments, the cell-penetrating peptide can be linked via the C-terminal end to an RNA molecule. In further embodiments, the peptide can be linked internally via the peptide backbone or side chains to an RNA molecule.

In further embodiments, the RNA complex is introduced into an insect cell. In some embodiments the RNA complex confers insecticidal activity. Also provided are the polynucleotide sequences that encode the RNA molecule. The RNA molecules can be designed for RNAi to downregulate the expression of a target mRNA of an insect pest. In other aspects, the RNAi-mediating molecules contain target polynucleotide sequences that specifically inhibit transcribed RNA from an expressed gene within an insect pest. For example, the target polynucleotide of the RNAi-mediating molecules inhibits a target gene by suppressing the expression of the mRNA of the target gene. In a further aspect, the stem structure of the RNAi-mediating molecules may be any polynucleotide sequence that shares at least 70% to 100% sequence identity with a target polynucleotide of a plant pest. For instance the polynucleotide may share at least 70% sequence identity, 71% sequence identity, 72% sequence identity, 73% sequence identity, 74% sequence identity, 75% sequence identity, 76% sequence identity, 77% sequence identity, 78% sequence identity, 79% sequence identity, 80% sequence identity, 81% sequence identity, 82% sequence identity, 83% sequence identity, 84% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, 99% sequence identity, 99.5% sequence identity, 99.9% sequence identity or 100% sequence identity with a target polynucleotide of an insect pest. In examples of this aspect the target polynucleotide may be an essential gene of the insect pest. Accordingly, the target polynucleotide sequence is obtained from a plant pest and is incorporated as a stem structure of 16-23 polynucleotides in length within the RNAi-mediating molecules. Similarly, the target polynucleotide sequence is obtained from a plant pest and is incorporated as a stem structure of 16-25 polynucleotides in length within the RNAi-mediating molecules. In other aspects, the stem structure may be a target polynucleotide that is selected from the group consisting of a Caf1-180 gene, RPA70 gene, V-ATPase H gene, Rho1 gene, V-ATPase C gene, Reptin gene, PPI-87B gene, RPS6 gene, COPI gamma gene, COPI alpha gene, COPI beta gene, COPI delta gene, Brahma gene, ROP gene, Hunchback gene, RNA polymerase II 140 gene, Sec23 gene, Dre4 gene, Gho gene, thread gene, ncm gene, RNA polymerase II-215 gene, RNA polymerase I 1 gene, RNA polymerase II 33 gene, Kruppel gene, Spt5 gene, Spt6 gene, Snap25 gene, SSJ1 gene, CoatG gene, and Prp8 gene. In further examples of this aspect the target polynucleotide sequences of the stem structure are selected from target gene homologs that were identified in the transcriptome sequence database as described in the following patent applications: U.S. Patent Application No. 20120174258; U.S. Patent Application No. 20130091601; U.S. Patent Application No. 20120198586; U.S. Patent Application No. US20120174260; U.S. Patent Application No. 20120174259; U.S. Patent Application No. 20140298536; U.S. Patent Application No. 20130091600; U.S. Patent Application No. 20130097730; Patent Application No. WO2016060911; Patent Application No. WO2016060912; Patent Application No. WO2016060913; Patent Application No. WO2016060914; U.S. Patent Application No. 20160208251; U.S. Patent Application No. 20160222408; U.S. Patent Application No. 20150176025; U.S. Patent Application No. 20160222407; U.S. Patent Application No. 20160208252; U.S. Patent Application No. 20150176009; U.S. Patent Application No. 20150322455; U.S. Patent Application No. 20150322456; U.S. Patent Application No. 20160186203; Patent Application No. WO2016191357; U.S. Patent Application No. 20160194658; U.S. Patent Application No. 20160264992; U.S. Patent Application No. 20160264991; U.S. Patent Application No. 20160355841; U.S. Patent Application No. 20160208253; U.S. Patent Application No. 20160369296; U.S. Patent Application No. 20160348130; U.S. Patent Application No. 2016196241; U.S. Patent Application No. 2017011764; and, U.S. Patent Application No. 2017011771. Such insect pests can include an insect that damages any economically important agronomic, forest, greenhouse, nursery ornamentals, food and fiber, public and animal health, domestic and commercial structure, household and stored product. In other aspects the RNA complex provides toxic insecticidal activity against one or more insect pests. Examples of such insect pests include, but is not limited to, members of the Lepidoptera, Diptera, Hemiptera and Coleoptera orders or the Nematoda phylum. In some embodiments, the insecticidal activity is provided against Lepidopteran, Dipteran, Heteropteran, Nematode, Hemiptera or Coleopteran pests. In further aspects of this embodiment, the Lepidopteran, Dipteran, Heteropteran, Nematode, Hemiptera or Coleopteran pests may be killed or reduced in numbers by the methods of the disclosure.

In other embodiments of the subject disclosure, methods are provided for producing the RNA complex and for using the RNA complex to control, inhibit growth or kill a Lepidopteran, Coleopteran, Nematode, Hemipteran and/or Dipteran pest. In some embodiments, the transgenic plants of the subject disclosure are engineered to express one or more polynucleotides encoding the polynucleotide as disclosed herein. In various embodiments, the transgenic plants further comprise one or more additional genes for insect resistance, for example, one or more additional genes for controlling Coleopteran, Lepidopteran, Hemipteran, Dipteran, and/or Nematode pests.

Exemplary aspects include use of the RNAi-mediating molecules in controlling, inhibiting growth or killing Lepidopteran, Dipteran, Heteropteran, Nematode, Hemiptera or Coleopteran pest populations and for producing compositions with insecticidal activity against such insects. Included as insect pests of interest are adults and nymphs.

Agronomically important species of interest from the order Lepidoptera include, but are not limited to, armyworms, cutworms, loopers, and heliothines in the family Noctuidae Spodoptera frugiperda JE Smith (fall armyworm); S. exigua Hubner (beet armyworm); S. litura Fabricius (tobacco cutworm, cluster caterpillar); Mamestra configurata Walker (bertha armyworm); M brassicae Linnaeus (cabbage moth); Agrotis ipsilon Hufnagel (black cutworm); A. orthogonia Morrison (western cutworm); A. subterranea Fabricius (granulate cutworm); Alabama argillacea Hubner (cotton leaf worm); Trichoplusia ni Hubner (cabbage looper); Pseudoplusia includens Walker (soybean looper); Anticarsia gemmatalis Hubner (velvetbean caterpillar); Hypena scabra Fabricius (green cloverworm); Heliothis virescens Fabricius (tobacco budworm); Pseudaletia unipuncta Haworth (armyworm); Athetis mindara Barnes and Mcdunnough (rough skinned cutworm); Euxoa messoria Harris (darksided cutworm); Earias insulana Boisduval (spiny bollworm); E. vittella Fabricius (spotted bollworm); Helicoverpa armigera Hubner (American bollworm); H. zea Boddie (corn earworm or cotton bollworm); Melanchra picta Harris (zebra caterpillar); Egira (Xylomyges) curialis Grote (citrus cutworm); borers, casebearers, webworms, coneworms, and skeletonizers from the family Pyralidae Ostrinia nubilalis Hubner (European corn borer); Amyelois transitella Walker (naval orangeworm); Anagasta kuehniella Zeller (Mediterranean flour moth); Cadra cautella Walker (almond moth); Chilo suppressalis Walker (rice stem borer); C. partellus, (sorghum borer); Corcyra cephalonica Stainton (rice moth); Crambus caliginosellus Clemens (corn root webworm); C. teterrellus Zincken (bluegrass webworm); Cnaphalocrocis medinalis Guenee (rice leaf roller); Desmia funeralis Hübner (grape leaffolder); Diaphania hyalinata Linnaeus (melon worm); D. nitidalis Stoll (pickleworm); Diatraea grandiosella Dyar (southwestern corn borer), D. saccharalis Fabricius (surgarcane borer); Eoreuma loftini Dyar (Mexican rice borer); Ephestia elutella Hübner (tobacco (cacao) moth); Galleria mellonella Linnaeus (greater wax moth); Herpetogramma licarsisalis Walker (sod webworm); Homoeosoma electellum Hulst (sunflower moth); Elasmopalpus lignosellus Zeller (lesser cornstalk borer); Achroia grisella Fabricius (lesser wax moth); Loxostege sticticalis Linnaeus (beet webworm); Orthaga thyrisalis Walker (tea tree web moth); Maruca testulalis Geyer (bean pod borer); Plodia interpunctella Hubner (Indian meal moth); Scirpophaga incertulas Walker (yellow stem borer); Udea rubigalis Guenee (celery leaftier); and leafrollers, budworms, seed worms, and fruit worms in the family Tortricidae Acleris gloverana Walsingham (Western blackheaded budworm); A. variana Fernald (Eastern blackheaded budworm); Archips argyrospila Walker (fruit tree leaf roller); A. rosana Linnaeus (European leaf roller); and other Archips species, Adoxophyes orana Fischer von Rosslerstamm (summer fruit tortrix moth); Cochylis hospes Walsingham (banded sunflower moth); Cydia latiferreana Walsingham (filbertworm); C. pomonella Linnaeus (coding moth); Platynota flavedana Clemens (variegated leafroller); P. stultana Walsingham (omnivorous leafroller); Lobesia botrana Denis & Schiffermuller (European grape vine moth); Spilonota ocellana Denis & Schiffermuller (eyespotted bud moth); Endopiza viteana Clemens (grape berry moth); Eupoecilia ambiguella Hubner (vine moth); Bonagota salubricola Meyrick (Brazilian apple leafroller); Grapholita molesta Busck (oriental fruit moth); Suleima helianthana Riley (sunflower bud moth); Argyrotaenia spp.; Choristoneura spp.

Other selected agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guérin-Meneville (Chinese Oak Tussah Moth); Bombyx mori Linnaeus (Silkworm); Bucculatrix thurberiella Busck (cotton leaf perforator); Collas eurytheme Boisduval (alfalfa caterpillar); Datana integerrima Grote & Robinson (walnut caterpillar); Dendrolimus sibiricus Tschetwerikov (Siberian silk moth), Ennomos subsignaria Hübner (elm spanworm); Erannis tiliaria Harris (linden looper); Euproctis chrysorrhoea Linnaeus (browntail moth); Harrisina americana Guerin-Meneville (grapeleaf skeletonizer); Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Manduca quinquemaculata Haworth (five spotted hawk moth, tomato hornworm); M sexta Haworth (tomato hornworm, tobacco hornworm); Operophtera brumata Linnaeus (winter moth); Paleacrita vernata Peck (spring cankerworm); Papilio cresphontes Cramer (giant swallowtail orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval & Leconte (Southern cabbageworm); Sabulodes aegrotata Guenee (omnivorous looper); Schizura concinna J. E. Smith (red humped caterpillar); Sitotroga cerealella Olivier (Angoumois grain moth); Thaumetopoea pityocampa Schiffermuller (pine processionary caterpillar); Tineola bisselliella Hummel (webbing clothesmoth); Tuta absoluta Meyrick (tomato leafminer); Yponomeuta padella Linnaeus (ermine moth); Heliothis subflexa Guenee; Malacosoma spp. and Orgyia spp.

Agronomically important species of interest from the order Coleoptera including weevils from the families Anthribidae, Bruchidae, and Curculionidae (including, but not limited to: Anthonomus grandis Boheman (boll weevil); Lissorhoptrus oryzophilus Kuschel (rice water weevil); Sitophilus granarius Linnaeus (granary weevil); S. oryzae Linnaeus (rice weevil); Hypera punctata Fabricius (clover leaf weevil); Cylindrocopturus adspersus LeConte (sunflower stem weevil); Smicronyx fulvus LeConte (red sunflower seed weevil); S. sordidus LeConte (gray sunflower seed weevil); Sphenophorus maidis Chittenden (maize billbug)); flea beetles, cucumber beetles, rootworms, leaf beetles, potato beetles, and leafminers in the family Chrysomelidae (including, but not limited to: Leptinotarsa decemlineata Say (Colorado potato beetle); Diabrotica virgifera virgifera LeConte (western corn rootworm); D. barberi Smith & Lawrence (northern corn rootworm); D. undecimpunctata howardi Barber (southern corn rootworm); Chaetocnema pulicaria Melsheimer (corn flea beetle); Phyllotreta cruciferae Goeze (corn flea beetle); Colaspis brunnea Fabricius (grape colaspis); Oulema melanopus Linnaeus (cereal leaf beetle); Zygogramma exclamationis Fabricius (sunflower beetle)); beetles from the family Coccinellidae (including, but not limited to: Epilachna varivestis Mulsant (Mexican bean beetle)); chafers and other beetles from the family Scarabaeidae (including, but not limited to: Popillia japonica Newman (Japanese beetle); Cyclocephala borealis Arrow (northern masked chafer, white grub); C. immaculate Olivier (southern masked chafer, white grub); Rhizotrogus majalis Razoumowsky (European chafer); Phyllophaga crinita Burmeister (white grub); Ligyrus gibbosus De Geer (carrot beetle)); carpet beetles from the family Dermestidae; wireworms from the family Elateridae, Eleodes spp., Melanotus spp.; Conoderus spp.; Limonius spp.; Agriotes spp.; Ctenicera spp.; Aeolus spp.; bark beetles from the family Scolytidae and beetles from the family Tenebrionidae.

Agronomically important species from the order Diptera are of interest, including leafminers Agromyza parvicornis Loew (corn blotch leafminer); midges (including, but not limited to: Contarinia sorghicola Coquillett (sorghum midge); Mayetiola destructor Say (Hessian fly); Sitodiplosis mosellana Géhin (wheat midge); Neolasioptera murtfeldtiana Felt, (sunflower seed midge)); fruit flies (Tephritidae), Oscinella frit Linnaeus (fruit flies); maggots (including, but not limited to: Delia platura Meigen (seedcorn maggot); D. coarctata Fallen (wheat bulb fly); and other Delia spp., Meromyza americana Fitch (wheat stem maggot); Musca domestica Linnaeus (house flies); Fannia canicularis Linnaeus, F. femoralis Stein (lesser house flies); Stomoxys calcitrans Linnaeus (stable flies)); face flies, horn flies, blow flies, Chrysomya spp.; Phormia spp.; and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds); and other Brachycera, mosquitoes Aedes spp.; Anopheles spp.; Culex spp.; black flies Prosimulium spp.; Simulium spp.; biting midges, sand flies, sciarids, and other Nematocera.

gronomically important species of interest from the orders Hemiptera and Homoptera such as, but not limited to, adelgids from the family Adelgidae, plant bugs from the family Miridae, cicadas from the family Cicadidae, leafhoppers, Empoasca spp.; from the family Cicadellidae, planthoppers from the families Cixiidae, Flatidae, Fulgoroidea, lssidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Asterolecanidae, Coccidae, Dactylopiidae, Diaspididae, Eriococcidae ortheziidae, Phoenicococcidae and Margarodidae, lace bugs from the family Tingidae, stink bugs from the family Pentatomidae, cinch bugs, Blissus spp.; and other seed bugs from the family Lygaeidae, spittlebugs from the family Cercopidae squash bugs from the family Coreidae, and red bugs and cotton stainers from the family Pyrrhocoridae.

Other agronomically important members from the order Homoptera further include, but are not limited to: Acyrthisiphon pisum Harris (pea aphid); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacorthum solani Kaltenbach (foxglove aphid); Chaetosiphon fragaefolii Cockerell (strawberry aphid); Diuraphis noxia Kurdjumov/Mordvilko (Russian wheat aphid); Dysaphis plantaginea Paaserini (rosy apple aphid); Eriosoma lanigerum Hausmann (woolly apple aphid); Brevicoryne brassicae Linnaeus (cabbage aphid); Hyalopterus pruni Geoffroy (mealy plum aphid); Lipaphis erysimi Kaltenbach (turnip aphid); Metopolophium dirrhodum Walker (cereal aphid); Macrosiphum euphorbiae Thomas (potato aphid); Myzus persicae Sulzer (peach-potato aphid, green peach aphid); Nasonovia ribisnigri Mosley (lettuce aphid); Pemphigus spp. (root aphids and gall aphids); Rhopalosiphum maidis Fitch (corn leaf aphid); R. padi Linnaeus (bird cherry-oat aphid); Schizaphis graminum Rondani (greenbug); Siphaflava Forbes (yellow sugarcane aphid); Sitobion avenae Fabricius (English grain aphid); Therioaphis maculata Buckton (spotted alfalfa aphid); Toxoptera aurantii Boyer de Fonscolombe (black citrus aphid); and T. citricida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly); B. argentifolii Bellows & Perring (silverleaf whitefly); Dialeurodes citri Ashmead (citrus whitefly); Trialeurodes abutiloneus (bandedwinged whitefly) and T. vaporariorum Westwood (greenhouse whitefly); Empoasca fabae Harris (potato leafhopper); Laodelphax striatellus Fallen (smaller brown planthopper); Macrolestes quadrilineatus Forbes (aster leafhopper); Nephotettix cinticeps Uhler (green leafhopper); N. nigropictus Stil (rice leafhopper); Nilaparvata lugens Stil (brown planthopper); Peregrinus maidis Ashmead (corn planthopper); Sogatella furcifera Horvath (white-backed planthopper); Sogatodes orizicola Muir (rice delphacid); Typhlocyba pomaria McAtee (white apple leafhopper); Erythroneoura spp. (grape leafhoppers); Magicicada septendecim Linnaeus (periodical cicada); Icerya purchasi Maskell (cottony cushion scale); Quadraspidiotus perniciosus Comstock (San Jose scale); Planococcus citri Risso (citrus mealybug); Pseudococcus spp. (other mealybug complex); Cacopsylla pyricola Foerster (pear psylla); Trioza diospyri Ashmead (persimmon psylla).

Agronomically important species of interest from the order Hemiptera include, but are not limited to: Acrosternum hilare Say (green stink bug); Anasa tristis De Geer (squash bug); Blissus leucopterus leucopterus Say (chinch bug); Corythuca gossypii Fabricius (cotton lace bug); Cyrtopeltis modesta Distant (tomato bug); Dysdercus suturellus Herrich-Schaffer (cotton stainer); Euschistus servus Say (brown stink bug); E. variolarius Palisot de Beauvois (one-spotted stink bug); Graptostethus spp. (complex of seed bugs); Leptoglossus corculus Say (leaf-footed pine seed bug); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Lygocoris pabulinus Linnaeus (common green capsid); Nezara viridula Linnaeus (southern green stink bug); Oebalus pugnax Fabricius (rice stink bug); Oncopeltus fasciatus Dallas (large milkweed bug); Pseudatomoscelis seriatus Reuter (cotton fleahopper).

Furthermore, embodiments may be effective against Hemiptera such as, Calocoris norvegicus Gmelin (strawberry bug); Orthops campestris Linnaeus; Plesiocoris rugicollis Fallen (apple capsid); Cyrtopeltis modestus Distant (tomato bug); Cyrtopeltis notatus Distant (suckfly); Spanagonicus albofasciatus Reuter (whitemarked fleahopper); Diaphnocoris chlorionis Say (honeylocust plant bug); Labopidicola allii Knight (onion plant bug); Pseudatomoscelis seriatus Reuter (cotton fleahopper); Adelphocoris rapidus Say (rapid plant bug); Poecilocapsus lineatus Fabricius (four-lined plant bug); Nysius ericae Schilling (false chinch bug); Nysius raphanus Howard (false chinch bug); Nezara viridula Linnaeus (Southern green stink bug); Eurygaster spp.; Coreidae spp.; Pyrrhocoridae spp.; Tinidae spp.; Blostomatidae spp.; Reduviidae spp.; and Cimicidae spp.

Agronomically important species of interest from the order Acari (mites) such as Aceria tosichella Keifer (wheat curl mite); Petrobia latens Müller (brown wheat mite); spider mites and red mites in the family Tetranychidae, Panonychus ulmi Koch (European red mite); Tetranychus urticae Koch (two spotted spider mite); (T. mcdanieli McGregor (McDaniel mite); T. cinnabarinus Boisduval (carmine spider mite); T. turkestani Ugarov & Nikolski (strawberry spider mite); flat mites in the family Tenuipalpidae, Brevipalpus lewisi McGregor (citrus flat mite); rust and bud mites in the family Eriophyidae and other foliar feeding mites and mites important in human and animal health, i.e. dust mites in the family Epidermoptidae, follicle mites in the family Demodicidae, grain mites in the family Glycyphagidae, ticks in the order Ixodidae. Ixodes scapularis Say (deer tick); I. holocyclus Neumann (Australian paralysis tick); Dermacentor variabilis Say (American dog tick); Amblyomma americanum Linnaeus (lone star tick); and scab and itch mites in the families Psoroptidae, Pyemotidae, and Sarcoptidae. Insect pests of the order Thysanura are of interest, such as Lepisma saccharina Linnaeus (silverfish); Thermobia domestica Packard (firebrat).

Additional arthropod pests covered include: spiders in the order Araneae such as Loxosceles reclusa Gertsch & Mulaik (brown recluse spider); and the Latrodectus mactans Fabricius (black widow spider); and centipedes in the order Scutigeromorpha such as Scutigera coleoptrata Linnaeus (house centipede).

Nematodes include parasitic nematodes such as root-knot, cyst, and lesion nematodes, including Heterodera spp., Meloidogyne spp., and Globodera spp.; particularly members of the cyst nematodes, including, but not limited to, Heterodera glycines (soybean cyst nematode); Heterodera schachtii (beet cyst nematode); Heterodera avenae (cereal cyst nematode); and Globodera rostochiensis and Globodera pailida (potato cyst nematodes). Lesion nematodes include Pratylenchus spp.

Methods for measuring insecticidal activity are well-known in the art. See, for example, Czapla and Lang, (1990) J. Econ. Entomol. 83:2480-2485; Andrews, et al., (1988) Biochem. J 252:199-206; Marrone, et al., (1985) J. of Economic Entomology 78:290-293 and U.S. Pat. No. 5,743,477, all of which are herein incorporated by reference in their entirety. Generally, the RNA complex is mixed and used in feeding assays. See, for example Marrone, et al., (1985) J. of Economic Entomology 78:290-293. Such assays can include contacting plants with one or more pests and determining the plant's ability to survive and/or cause the death of the pests. For each substance or organism, the insecticidally effective amount is determined empirically for each pest affected in a specific environment.

Methods for Inhibiting or Killing an Insect Pest and Controlling an Insect Population.

In some embodiments methods are provided for inhibiting growth or killing an insect pest, comprising contacting the insect pest with an insecticidally-effective amount of the RNA complex. In certain aspects the RNA complex comprises RNAi-mediating molecules.

In some embodiments methods are provided for controlling an insect pest population, comprising contacting the insect pest population with an insecticidally-effective amount of the RNA complex. In certain aspects the RNA complex comprises RNAi-mediating molecules.

In some embodiments methods are provided for controlling an insect pest population resistant to a pesticidal RNAi molecules (e.g. small RNA molecules), comprising contacting the insect pest population with an insecticidally-effective amount of the RNA complex. In certain aspects the RNA complex comprises RNAi-mediating molecules.

In some embodiments methods are provided for protecting a plant from an insect pest, comprising expressing in the plant or cell thereof, the RNA complex. In certain aspects the RNA complex comprises RNAi-mediating molecules.

Insect Resistance Management (IRM) Strategies

One way to increase the effectiveness of transgenic insect resistance traits against target insect pests and contemporaneously reduce the development of insecticide-resistant pests is to use or provide non-transgenic (i.e., non-insecticidal protein or RNA complex) refuges (a section of non-insecticidal crops/corn). The United States Environmental Protection Agency (epa.gov/oppbppdl/biopesticides/pips/bt_corn_refuge-2006.htm, which can be accessed using the www prefix) publishes the requirements for use with transgenic crops producing a single Bt protein active against target pests. In addition, the National Corn Growers Association, on their website: (ncga.com/insect-resistance-management-fact-sheet-bt-corn, which can be accessed using the www prefix) also provides similar guidance regarding refuge requirements. Due to losses to insect pests within the refuge area, larger refuges may reduce overall yield.

Another way to increase the effectiveness of the transgenic insect resistance traits against target insect pests and contemporaneously reduce the development of insecticide-resistant pests would be to have a repository of insecticidal genes that are effective against groups of insect pests and which manifest their effects through different modes of action.

Expression in a plant of two or more insecticidal compositions toxic to the same insect species, each insecticide being expressed at efficacious levels would be another way to achieve control of the development of insect resistance to transgenic plants. This is based on the principle that evolution of resistance against two separate modes of action is far more unlikely than only one. Roush for example, outlines two-toxin strategies, also called “pyramiding” or “stacking,” for management of insecticidal transgenic crops. (The Royal Society. Phil. Trans. R. Soc. Lond. B. (1998) 353:777-1786). Stacking or pyramiding of two different insecticidal molecules each effective against the target pests and with little or no cross-resistance can allow for use of a smaller refuge. The U.S. Environmental Protection Agency requires significantly less (generally 5%) structured refuge of non-Bt corn be planted than for single trait products (generally 20%). There are various ways of providing the insect resistance management effects of a refuge, including various geometric planting patterns in the crop fields and in-bag seed mixtures, as discussed further by Roush.

In some embodiments the RNA complex of the subject disclosure is useful as an insect resistance management strategy in combination (i.e., pyramided) with other insecticidal molecules including but not limited to Bt toxins, Xenorhabdus sp. or Photorhabdus sp. insecticidal proteins, small RNA or RNAi-mediating molecules, and the like. In such an embodiment, the yield of the plant is significantly increased.

Provided are methods of controlling Lepidopteran, Dipteran, Heteropteran, Nematode, Hemiptera or Coleopteran insect infestation(s) in a transgenic plant that promote insect resistance management, comprising expressing in the plant at least two different insecticidal molecules having different modes of action. In certain aspects one of the insecticidal molecules comprises RNAi-mediating molecules. In such an embodiment, the yield of the plant is significantly increased.

In some embodiments the methods of controlling Lepidopteran, Dipteran, Heteropteran, Nematode, Hemiptera and/or Coleopteran insect infestation in a transgenic plant and promoting insect resistance management wherein at least one of the insecticidal molecules comprise an RNAi-mediating molecule with insecticidal activity to insects in the Order Lepidopteran, Dipteran, Heteropteran, Nematode, Hemiptera and/or Coleopteran. In such an embodiment, the yield of the plant is significantly increased.

In some embodiments the methods of controlling Lepidopteran, Dipteran, Heteropteran, Nematode, Hemiptera and/or Coleopteran insect infestation in a transgenic plant and promoting insect resistance management comprise expressing in the transgenic plant an RNA and a protein with insecticidal activity to insects in the order Lepidopteran, Dipteran, Heteropteran, Nematode, Hemiptera and/or Coleopteran having different modes of action. In such an embodiment, the yield of the plant is significantly increased.

Also provided are methods of reducing likelihood of emergence of Lepidopteran, Dipteran, Heteropteran, Nematode, Hemiptera and/or Coleopteran insect resistance to transgenic plants expressing insecticidal molecules to control the insect species, comprising expression of the RNA complex with insecticidal activity to the insect species in combination with a second insecticidal molecule to the insect species having different modes of action. In certain aspects the RNA complex comprises RNAi-mediating molecules. In such an embodiment, the yield of the plant is significantly increased.

Also provided herein are means for effective Lepidopteran, Dipteran, Heteropteran, Nematode, Hemiptera and/or Coleopteran insect resistance management of transgenic plants, comprising co-expressing in the plants two or more insecticidal molecules toxic to Lepidoptera and/or Hemiptera insects but each exhibiting a different mode of effectuating its inhibitory activity, wherein the two or more insecticidal molecules comprise the RNA complex and a Cry protein. In certain aspects the RNA complex comprises RNAi-mediating molecules.

In addition, methods are provided for obtaining regulatory approval for planting or commercialization of plants expressing molecules insecticidal to insects in the order Lepidopteran, Dipteran, Heteropteran, Nematode, Hemiptera and/or Coleopteran, comprising the step of referring to, submitting or relying on insect assay binding data showing that the RNA complex does not compete with binding sites for Cry proteins in such insects. In certain aspects the RNA complex comprises RNAi-mediating molecules. In addition, methods are provided for obtaining regulatory approval for planting or commercialization of plants expressing molecules insecticidal to insects in the order Lepidopteran, Dipteran, Heteropteran, Nematode, Hemiptera and/or Coleopteran, comprising the step of referring to, submitting or relying on insect assay binding data showing that the RNA complex does not compete with binding sites for Cry proteins in such insects. In such an embodiment, the yield of the plant is significantly increased.

The use of Cry proteins as transgenic plant traits is well-known to one skilled in the art and Cry-transgenic plants including but not limited to Cry1Ac, Cry1Ac+Cry2Ab, Cry1Ab, Cry1A.105, Cry1F, Cry1Fa2, Cry1F+Cry1Ac, Cry2Ab, Cry3A, mCry3A, Cry3Bb1, Cry34Ab1, Cry35Ab1, Vip3A, mCry3A, Cry9c and CBI-Bt have received regulatory approval (see, Sanahuja, (2011) Plant Biotech Journal 9:283-300 and the CERA (2010) GM Crop Database Center for Environmental Risk Assessment (CERA), ILSI Research Foundation, Washington D.C. at cera-gmc.org/index.php?action=gm_crop_database which can be accessed on the world-wide web using the “www” prefix). More than one insecticidal molecules well-known to one skilled in the art can also be expressed in plants such as Vip3Ab & Cry1Fa (US2012/0317682), Cry1BE & Cry1F (US2012/0311746), Cry1CA & Cry1AB (US2012/0311745), Cry1F & CryCa (US2012/0317681), Cry1DA & Cry1BE (US2012/0331590), Cry1DA & Cry1Fa (US2012/0331589), Cry1AB & Cry1BE (US2012/0324606), and Cry1Fa & Cry2Aa, Cry1I or Cry1E (US2012/0324605).

Insecticidal molecules also include insecticidal lipases including lipid acyl hydrolases of U.S. Pat. No. 7,491,869, and cholesterol oxidases such as from Streptomyces (Purcell et al. (1993) Biochem Biophys Res Commun 15:1406-1413). Insecticidal molecules further include IPD072 (PCT/US14/55128), and IPD079 (PCT/US2016/041452). Insecticidal molecules also include VIP (vegetative insecticidal proteins) toxins of U.S. Pat. Nos. 5,877,012, 6,107,279, 6,137,033, 7,244,820, 7,615,686, and 8,237,020, and the like. Other VIP proteins are well-known to one skilled in the art (see, lifesci.sussex.ac.uk/home/Neil_Crickmore/Bt/vip.html which can be accessed on the world-wide web using the “www” prefix). Insecticidal molecules also include toxin complex (TC) proteins, obtainable from organisms such as Xenorhabdus, Photorhabdus and Paenibacillus (see, U.S. Pat. Nos. 7,491,698 and 8,084,418). Some TC proteins have “stand alone” insecticidal activity and other TC proteins enhance the activity of the stand-alone toxins produced by the same given organism. The toxicity of a “stand-alone” TC protein (from Photorhabdus, Xenorhabdus or Paenibacillus, for example) can be enhanced by one or more TC protein “potentiators” derived from a source organism of a different genus. There are three main types of TC proteins. As referred to herein, Class A proteins (“Protein A”) are stand-alone toxins. Class B proteins (“Protein B”) and Class C proteins (“Protein C”) enhance the toxicity of Class A proteins. Examples of Class A proteins are TcbA, TcdA, XptA1 and XptA2. Examples of Class B proteins are TcaC, TcdB, XptB1Xb and XptC1Wi. Examples of Class C proteins are TccC, XptC1Xb and XptB1Wi. Insecticidal molecules also include spider, snake and scorpion venom proteins. Examples of spider venom peptides include but are not limited to lycotoxin-1 peptides and mutants thereof (U.S. Pat. No. 8,334,366). In such an embodiment, the yield of the plant is significantly increased.

In a further embodiment, a method of expressing a gene encoding the RNA complex within a plant results in protecting the plant from an insect pest via suppressing the expression of the target mRNA of an insect pest. In certain aspects the RNA complex comprises RNAi-mediating molecules. In such an embodiment, the yield of the plant is significantly increased.

Also disclosed herein are methods for delivery of RNA complex to an insect pest. Such control agents may cause, directly or indirectly, an impairment in the ability of the insect to feed, grow, or otherwise cause damage on a host plant. In some embodiments, a method is provided comprising delivery of a RNA complex to an insect pest to suppress at least one target gene in the insect pest, thereby reducing or eliminating plant damage by the insect pest. In some embodiments, a method of inhibiting expression of a target gene in the insect pest may result in the cessation of growth, development, reproduction, and/or feeding in the insect pest. In some embodiments, the method may eventually result in death of the insect pest.

In some embodiments, compositions (e.g., a topical composition) are provided that comprise the RNA complex of the disclosure for use in plants, and/or the environment of a plant to achieve the elimination or reduction of the insect pest. In particular embodiments, the composition may be a nutritional composition or food source to be up taken by the insect pest. Some embodiments comprise making the nutritional composition or food source available to the insect pest. Uptake of a composition comprising the RNA complex may result in the uptake of the molecules by one or more cells of the insect pest, which may in turn result in the inhibition of expression of at least one target gene in cell(s) of the insect pest. Uptake of or damage to a plant or plant cell by an insect pest may be limited or eliminated in or on any host tissue or environment in which the insect pest is present by providing one or more compositions comprising an RNA complex of the disclosure in the host of the insect pest.

In other embodiments, the composition may be a topical composition. Some embodiments comprise making the topical composition available to the insect pest. Contact of a composition comprising the RNA complex of the subject disclosure may result in the uptake of the molecules by one or more cells of the insect pest, which may in turn result in the inhibition of expression of at least one target gene in cell(s) of the insect pest. Damage to a plant or plant cell by an insect pest may be limited or eliminated in or on any host tissue or environment in which the insect pest is present by providing one or more compositions comprising the RNA complex of the disclosure in the host of the insect pest.

In an embodiment, the mRNA molecule of interest is a polynucleotide. In certain aspects, the polynucleotide encodes a gene. In some aspects, the molecule of interest is a heterologous coding sequence (for example, a transgene of interest). Transgenes of interest may be complexed to the cell-penetrating peptide of the subject disclosure. Exemplary transgenes of interest that are suitable for use in the present disclosed constructs include, but are not limited to, coding sequences that confer (1) resistance to pests or disease, (2) tolerance to herbicides, (3) value-added agronomic traits, such as; yield improvement, nitrogen use efficiency, water use efficiency, and nutritional quality, (4) binding of a protein to DNA in a site-specific manner, (5) expression of small RNA or RNAi-mediating molecule, and (6) selectable markers. In accordance with one embodiment, the cell-penetrating peptide is complexed with a mRNA molecule of interest to deliver a transgene/heterologous coding sequence encoding a selectable marker or a gene product conferring insecticidal resistance, herbicide tolerance, small RNA or RNAi-mediating molecule expression, nitrogen use efficiency, water use efficiency, or nutritional quality.

1. Insect Resistance

Various insect resistance genes can be linked to the cell-penetrating peptides as an mRNA. The cell-penetrating peptide can be operably linked to an mRNA that expresses an insect resistance trait. The operably linked sequences can then be incorporated into a chosen vector to allow for the identification and selection of transformed plants (“transformants”). Exemplary insect resistance coding sequences are known in the art. As embodiments of insect resistance coding sequences that can be operably linked to the regulatory elements of the subject disclosure, the following traits are provided.

Coding sequences that provide exemplary Lepidopteran insect resistance include cry1A; cry1A.105; cry1Ab; cry1Ab(truncated); cry1Ab-Ac (fusion protein); cry1Ac (marketed as Widestrike®); cry1C; cry1F (marketed as Widestrike®); cry1Fa2; cry2Ab2; cry2Ae; cry9C; mocryiF; pinII (protease inhibitor protein); vip3A(a); and vip3Aa20. Coding sequences that provide exemplary Coleopteran insect resistance include: cry34Ab1 (marketed as Herculex®); cry35Ab1 (marketed as Herculex®); cry3A; cry3Bb1; dvsnf7; and mcry3A. Coding sequences that provide exemplary Hemipteran resistance include mCry51Aa2. Coding sequences that provide exemplary multi-insect resistance include ecry31.Ab. The above list of insect resistance genes is not meant to be limiting. Any insect resistance genes are encompassed by the present disclosure.

2. Herbicide Tolerance

Various herbicide tolerance genes be can be linked to the cell-penetrating peptides as an mRNA. The cell-penetrating peptide can be operably linked to an mRNA that expresses an herbicide tolerance trait. The operably linked sequences can then be incorporated into a chosen vector to allow for the identification and selection of transformed plants (“transformants”). Exemplary herbicide tolerance coding sequences are known in the art. As embodiments of herbicide tolerance coding sequences that can be operably linked to the regulatory elements of the subject disclosure, the following traits are provided. The glyphosate herbicide contains a mode of action of inhibiting the EPSPS enzyme (5-enolpyruvylshikimate-3-phosphate synthase). This enzyme is involved in the biosynthesis of aromatic amino acids that are essential for the growth and development of plants. Various enzymatic mechanisms are known in the art that can be utilized to inhibit this enzyme. The genes that encode such enzymes can be operably linked to the gene regulatory elements of the subject disclosure. In an embodiment, selectable marker genes include, but are not limited to genes encoding glyphosate resistance genes include mutant EPSPS genes such as 2mEPSPS genes, cp4 EPSPS genes, mEPSPS genes, dgt-28 genes; aroA genes; and glyphosate degradation genes such as glyphosate acetyltransferase genes (gat) and glyphosate oxidase genes (gox). These traits are currently marketed as Gly-Tol™, Optimum® GAT®, Agrisure® GT, and Roundup Ready®. Resistance genes for glufosinate and/or bialaphos compounds include dsm-2, bar, and pat genes. The bar and pat traits are currently marketed as LibertyLink®. Also included are tolerance genes that provide resistance to 2,4-D such as aad-1 genes (it should be noted that aad-1 genes have further activity on aryloxyphenoxypropionate herbicides) and aad-12 genes (it should be noted that aad-12 genes have further activity on pyidyloxyacetate synthetic auxins). These traits are marketed as Enlist® crop protection technology. Resistance genes for ALS inhibitors (sulfonylureas, imidazolinones, triazolopyrimidines, pyrimidinylthiobenzoates, and sulfonylamino-carbonyl-triazolinones) are known in the art. These resistance genes most commonly result from point mutations to the ALS encoding gene sequence. Other ALS inhibitor resistance genes include hra genes, the csrl-2 genes, Sr-HrA genes, and surB genes. Some of the traits are marketed under the tradename Clearfield®. Herbicides that inhibit HPPD include the pyrazolones such as pyrazoxyfen, benzofenap, and topramezone; triketones such as mesotrione, sulcotrione, tembotrione, benzobicyclon; and diketonitriles such as isoxaflutole. These exemplary HPPD herbicides can be tolerated by known traits. Examples of HPPD inhibitors include hppdPF_W336 genes (for resistance to isoxaflutole) and avhppd-03 genes (for resistance to meostrione). An example of Bromoxynil herbicide-tolerant traits include the bxn gene, which has been showed to impart resistance to the herbicide/antibiotic bromoxynil. Resistance genes for dicamba include the dicamba monooxygenase gene (dmo) as disclosed in International PCT Publication No. WO 2008/105890. Resistance genes for PPO or PROTOX inhibitor type herbicides (e.g., acifluorfen, butafenacil, flupropazil, pentoxazone, carfentrazone, fluazolate, pyraflufen, baclofen, azafenidin, flumioxazin, flumiclorac, biphenol, oxyfluorfen, lactofen, fomesafen, fluoroglycofen, and sulfentrazone) are known in the art. Exemplary genes conferring resistance to PPO include overexpression of a wild-type Arabidopsis thaliana PPO enzyme (Lermontova I and Grimm B, (2000) Overexpression of plastidic protoporphyrinogen IX oxidase leads to resistance to the diphenyl-ether herbicide acifluorfen. Plant Physiol 122:75-83.), the B. subtilis PPO gene (Li, X. and Nicholl D. 2005. Development of PPO inhibitor-resistant cultures and crops. Pest Manag. Sci. 61:277-285 and Choi KW, Han O, Lee H J, Yun Y C, Moon Y H, Kim M K, Kuk Y I, Han S U and Guh J O, (1998).Generation of resistance to the diphenyl ether herbicide, oxyfluorfen, via expression of the Bacillus subtilis protoporphyrinogen oxidase gene in transgenic tobacco plants. Biosci Biotechnol Biochem 62:558-560.) Resistance genes for pyridinoxy or phenoxy proprionic acids and cyclohexones include the ACCase inhibitor-encoding genes (e.g., Acc1-S1, Acc1-S2, and Acc1-S3). Exemplary genes conferring resistance to cyclohexanediones and/or aryloxyphenoxypropanoic acid include haloxyfop, diclofop, fenoxyprop, fluazifop, and quizalofop. Finally, herbicides can inhibit photosynthesis, including triazine or benzonitrile are provided tolerance by psbA genes (tolerance to triazine), Is+ genes (tolerance to triazine), and nitrilase genes (tolerance to benzonitrile). The above list of herbicide tolerance genes is not meant to be limiting. Any herbicide tolerance genes are encompassed by the present disclosure.

3. Agronomic Traits

Various agronomic trait genes can be linked to the cell-penetrating peptides as an mRNA. The cell-penetrating peptide can be operably linked to an mRNA as an agronomic trait gene. The operably linked sequences can then be incorporated into a chosen vector to allow for the identification and selection of transformed plants (“transformants”). Exemplary agronomic trait coding sequences are known in the art. As embodiments of agronomic trait coding sequences that can be operably linked to the regulatory elements of the subject disclosure, the following traits are provided. Increased ear biomass in Zea mays as provided by the athb17 genes can result in greater ear size and enhanced silk potential when expressed in Zea mays. Delayed fruit softening as provided by the pg genes inhibit the production of polygalacturonase enzyme responsible for the breakdown of pectin molecules in the cell wall and thus causes delayed softening of the fruit.

Further, delayed fruit ripening/senescence of acc genes act to suppress the normal expression of the native acc synthase gene, resulting in reduced ethylene production and delayed fruit ripening. Whereas, the accd genes metabolize the precursor of the fruit ripening hormone ethylene, resulting in delayed fruit ripening. Alternatively, the sam-k genes cause delayed ripening by reducing S-adenosylmethionine (SAM), a substrate for ethylene production. Drought stress tolerance phenotypes, as provided by cspB genes, maintain normal cellular functions under water stress conditions by preserving RNA stability and translation. A further example includes Hahb-4 genes. Another example includes the EcBetA genes that catalyze the production of the osmoprotectant compound glycine betaine conferring tolerance to water stress. In addition, the RmBetA genes catalyze the production of the osmoprotectant compound glycine betaine conferring tolerance to water stress. Photosynthesis and yield enhancement are provided with the bbx32 gene that expresses a protein that interacts with one or more endogenous transcription factors to regulate the plant's day/night physiological processes. Ethanol production can be increased by expression of the amy797E genes that encode a thermostable alpha-amylase enzyme that enhances bioethanol production by increasing the thermostability of amylase used in degrading starch. Finally, modified amino acid compositions can result in the expression of the cordapA genes that encode a dihydrodipicolinate synthase enzyme that increases the production of amino acid lysine. The above list of agronomic trait coding sequences is not meant to be limiting. Any agronomic trait coding sequence is encompassed by the present disclosure.

4. DNA Binding Protein

Various DNA binding transgene/heterologous coding sequences can be linked to the cell-penetrating peptides as an mRNA. The cell-penetrating peptide can be operably linked as an mRNA that expresses a DNA binding gene trait. The operably linked sequences can then be incorporated into a chosen vector to allow for identification and selectable of transformed plants (“transformants”).

Exemplary DNA binding protein-coding sequences are known in the art. As embodiments of DNA binding protein-coding sequences that can be operably linked to the regulatory elements of the subject disclosure, the following types of DNA binding proteins can include; Zinc Fingers, TALENS, CRISPRS, and meganucleases. The above list of DNA binding protein-coding sequences is not meant to be limiting. Any DNA binding protein-coding sequences are encompassed by the present disclosure.

5. Small RNA

Various small RNA sequences can be linked to the cell-penetrating peptides as an mRNA. The cell-penetrating peptide can be operably linked to an mRNA that expresses a small RNA sequence trait. The operably linked sequences can then be incorporated into a chosen vector to allow for the identification and selection of transformed plants (“transformants”). Exemplary small RNA traits are known in the art. As embodiments of small RNA coding sequences that can be operably linked to the regulatory elements of the subject disclosure, the following traits are provided. For example, delayed fruit ripening/senescence of the anti-efe small RNA delays ripening by suppressing the production of ethylene via silencing of the ACO gene that encodes an ethylene-forming enzyme. The altered lignin production of ccomt small RNA reduces the content of guanacyl (G) lignin by inhibition of the endogenous S-adenosyl-L-methionine: trans-caffeoyl CoA 3-O-methyltransferase (CCOMT gene).

Further, the Black Spot Bruise Tolerance in Solanum verrucosum can be reduced by the Ppo5 small RNA, which triggers the degradation of Ppo5 transcripts to block black spot bruise development. Also included is the dvsnf7 small RNA that inhibits Western Corn Rootworm with dsRNA containing a 240 bp fragment of the Western Corn Rootworm Snf7 gene. Modified starch/carbohydrates can result from small RNA, such as the pPhL small RNA (degrades PhL transcripts to limit the formation of reducing sugars through starch degradation) and pR1 small RNA (degrades R1 transcripts to limit the formation of reducing sugars through starch degradation). Additional benefits such as reduced acrylamide resulting from the asn1 small RNA that triggers degradation of Asn1 to impair asparagine formation and reduce polyacrylamide. Finally, the non-browning phenotype of pgas ppo suppression small RNA results in suppressing PPO to produce apples with a non-browning phenotype. The above list of small RNAs is not meant to be limiting. Any small RNA encoding sequences are encompassed by the present disclosure.

6. Selectable Markers

Various selectable markers also described as reporter genes can be linked to the cell-penetrating peptides. The cell-penetrating peptide can be operably linked to an mRNA that expresses the reporter gene trait. The operably linked sequences can then be incorporated into a chosen vector to allow for identification and selection of transformed plants (“transformants”). Many methods are available to confirm the expression of selectable markers in transformed plants, including for example DNA sequencing and PCR (polymerase chain reaction), Southern blotting, Northern blotting, immunological methods for the detection of a protein expressed from the vector. But, usually, the reporter genes are observed through visual observation of proteins that, when expressed, produce a colored product. Exemplary reporter genes are known in the art and encode/-glucuronidase (GUS), luciferase, a greenfluorescent protein (GFP), a yellow fluorescent protein (YFP, Phi-YFP), a red fluorescentprotein (DsRFP, RFP, etc.), β-galactosidase, and the like (See Sambrook, et al., Molecular Cloning: A Laboratory Manual, Third Edition, Cold Spring Harbor Press, N.Y., 2001, the content of which is incorporated herein by reference in its entirety).

Selectable marker genes are utilized for the selection of transformed cells or tissues. Selectable marker genes include genes encoding antibiotic resistance, such as those encoding neomycin phosphotransferase II (NEO), spectinomycin/streptomycin resistance (AAD), and hygromycin phosphotransferase (HPT or HGR) as well as genes conferring resistance to herbicidal compounds. Herbicide resistance genes generally code for a modified target protein insensitive to the herbicide or for an enzyme that degrades or detoxifies the herbicide in the plant before it can act. For example, resistance to glyphosate has been obtained by using genes coding for mutant target enzymes, 5-enolpyruvylshikimate-3-phosphate synthase (EPSPS). Genes and mutants for EPSPS are well-known, and further described below. Resistance to glufosinate-ammonium, bromoxynil, and 2,4-dichlorophenoxyacetate (2,4-D) have been obtained by using bacterial genes encoding PAT or DSM-2, a nitrilase, an AAD-1, or an AAD-12, each of which are examples of proteins that detoxify their respective herbicides.

In an embodiment, herbicides can inhibit the growing point or meristem, including imidazolinone or sulfonylurea, and genes for resistance/tolerance of acetohydroxyacid synthase (AHAS) and acetolactate synthase (ALS) for these herbicides are well-known. Glyphosate resistance genes include mutant 5-enolpyruvylshikimate-3-phosphate synthase (EPSPs) and dgt-28 genes (via the introduction of recombinant nucleic acids and/or various forms of in vivo mutagenesis of native EPSPs genes), aroA genes and glyphosate acetyltransferase (GAT) genes, respectively). Resistance genes for other phosphono compounds include bar and pat genes from Streptomyces species, including Streptomyces hygroscopicus and Streptomyces viridichromogenes, and pyridinoxy or phenoxy proprionic acids and cyclohexones (ACCase inhibitor-encoding genes). Exemplary genes conferring resistance to cyclohexanediones and/or aryloxyphenoxypropanoic acid (including haloxyfop, diclofop, fenoxyprop, fluazifop, quizalofop) include genes of acetyl coenzyme A carboxylase (ACCase); Acc1-S1, Acc1-S2, and Acc1-S3. In an embodiment, herbicides can inhibit photosynthesis, including triazine (psbA and 1s+ genes) or a benzonitrile (nitrilase gene). Furthermore, such selectable markers can include positive selection markers such as phosphomannose isomerase (PMI) enzyme.

In an embodiment, selectable marker genes include, but are not limited to genes encoding: 2,4-D; neomycin phosphotransferase II; cyanamide hydratase; aspartate kinase; dihydrodipicolinate synthase; tryptophan decarboxylase; dihydrodipicolinate synthase and desensitized aspartate kinase; bar gene; tryptophan decarboxylase; neomycin phosphotransferase (NEO); hygromycin phosphotransferase (HPT or HYG); dihydrofolate reductase (DHFR); phosphinothricin acetyltransferase; 2,2-dichloropropionic acid dehalogenase; acetohydroxyacid synthase; 5-enolpyruvyl-shikimate-phosphate synthase (aroA); haloarylnitrilase; acetyl-coenzyme A carboxylase; dihydropteroate synthase (sul I); and 32 kD photosystem II polypeptide (psbA). An embodiment also includes selectable marker genes encoding resistance to chloramphenicol; methotrexate; hygromycin; spectinomycin; bromoxynil; glyphosate; and phosphinothricin. The above list of selectable marker genes is not meant to be limiting. Any reporter or selectable marker gene are encompassed by the present disclosure.

In some embodiments, the coding sequences are synthesized for optimal expression in a plant. For example, in an embodiment, a coding sequence of a gene has been modified by codon optimization to enhance expression in plants. An insecticidal resistance transgene, an herbicide tolerance transgene, a nitrogen use efficiency transgene, a water use efficiency transgene, a nutritional quality transgene, a DNA binding transgene, or a selectable marker transgene/heterologous coding sequence can be optimized for expression in a particular plant species or alternatively can be modified for optimal expression in dicotyledonous or monocotyledonous plants. Plant-preferred codons may be determined from the codons of highest frequency in the proteins expressed in the largest amount in the particular plant species of interest. In an embodiment, a coding sequence, gene, heterologous coding sequence or transgene/heterologous coding sequence is designed to be expressed in plants at a higher level resulting in higher transformation efficiency. Methods for plant optimization of genes are well-known. Guidance regarding the optimization and production of synthetic DNA sequences can be found in, for example, WO2013016546, WO2011146524, WO1997013402, U.S. Pat. Nos. 6,166,302, and 5,380,831, US Patent Application No. 20140090115, herein incorporated by reference.

In embodiments of the subject disclosure, the disclosure relates to a gene expression cassette engineered within a vector. Examples of a vector include a plasmid, a cosmid, a bacterial artificial chromosome, a virus, and a bacteriophage. In an aspect the gene expression cassette comprises one or more additional transgenic traits. In another aspect the one or more additional transgenic traits is selected from the group consisting of a heterologous coding sequence conferring insecticidal resistance, herbicide tolerance, a nucleic acid conferring nitrogen use efficiency, a nucleic acid conferring water use efficiency, a nucleic acid conferring nutritional quality, a nucleic acid encoding a DNA binding protein, and a nucleic acid encoding a selectable marker. In other aspects the heterologous coding sequence is operably linked to one or more heterologous regulatory sequences that drive expression of the RNA complex.

Commodity Product

In an embodiment, the subject disclosure includes a commodity product. In certain aspects the commodity product is produced within the transgenic plant of the subject disclosure. Exemplary commodity products include protein concentrate, protein isolate, grain, meal, flour, oil, or fiber. In other examples such commodity products may include whole or processed seeds, animal feed containing transgenic plants of the subject disclosure or transgenic plant by-products, oil, meal, flour, starch, flakes, bran, biomass and stover, and fuel products and fuel by-products when made from transgenic plants or plant parts.

Furthermore, the commodity products may be sold to consumers and may be viable or nonviable. Nonviable commodity products include but are not limited to nonviable seeds; processed seeds, seed parts, and plant parts; seeds and plant parts processed for feed or food, oil, meal, flour, flakes, bran, biomasses, and fuel products. Viable commodity products include but are not limited to seeds, plants, and plant cells. The plants comprising the polynucleotides and RNA complex of the subject disclosure can thus be used to manufacture any commodity product typically acquired from such a transgenic crop plant.

Crop Plants

As used herein, the term “plant” includes a whole plant and any descendant, cell, tissue, or part of a plant. A class of plant that can be used in the present invention is generally as broad as the class of higher and lower plants amenable to mutagenesis including angiosperms (monocotyledonous and dicotyledonous plants), gymnosperms, ferns and multicellular algae. Thus, “plant” includes dicot and monocot plants. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf, stem; fruit; seed; and root.

A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete-producing cell, or a cell or collection of cells that can regenerate into a whole plant. As such, a seed, which comprises multiple plant cells and is capable of regenerating into a whole plant, is considered a “plant cell” in embodiments herein.

All references, including publications, patents, and patent applications, cited herein are hereby incorporated by reference to the extent they are not inconsistent with the explicit details of this disclosure and are so incorporated to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention.

Embodiments of the subject disclosure are further exemplified in the following Examples. It should be understood that these examples are given by way of illustration only. From the above embodiments and the following Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims. The following is provided by way of illustration and not intended to limit the scope of the invention.

### EXAMPLES

### Example 1: Preparation of RNAs

Unlabeled single-stranded messenger RNA (mRNA) encoding the mCherry fluorescent protein (CleanCap mCherry mRNA [5moU], 1.0 mg·mL⁻¹ in 1 mM Sodium Citrate, pH 6.4) was obtained from TriLink Biotechnologies (San Diego, CA). Unlabeled double-stranded RNA (dsRNA) representing dvssj1 frag 1 of length 210 base pairs (bp) was obtained from Genolution Inc. (Seoul, Korea) (see description of sequence and activity in Hu et al. (2016) “Discovery of midgut genes for the RNA interference control of corn rootworm”, Scientific Reports 6:30542, DOI: 10.1038/srep30542). Final unlabeled dsRNA concentration and purity was determined as 2.1 mg·mL⁻¹ and 95.1%, respectively, and exhibited a single band by gel electrophoresis and single peak by dynamic light scattering (DLS) (FIG. 1A). The highest-abundance small interfering RNA (siRNA) produced from western corn rootworm (WCR) oral ingestion and processing of dvssj1 frag 1 was ordered from Integrated DNA Technologies (Coralville, IA) with one molecule of Cy3 dye conjugated to each 5′ end (Table 3). DsRNA targeting dvssj1 frag 1 was fluorescently labeled with Cy3 dye during in vitro transcription using the primers described in Table 3 with Invitrogen™ MEGAscript® T7 Transcription Kit (Thermo Fisher Scientific, Waltham, MA) according to manufacturer's instructions, replacing 83% of CTP and UTP with Cy3-CTP and Cy3-UTP nucleotides from Cytiva (Marlborough, MA). Labeled RNA was purified using the Invitrogen™ MEGAclear™ Transcription Clean-Up Kit (Thermo Fisher Scientific) according to manufacturer's instructions. Final labeled dsRNA concentration was determined as 595.8 ng·μL⁻¹ and exhibited a single band by gel electrophoresis and single peak by DLS (FIG. 1).

TABLE 3
Sequences used for generating Cy3-labeled test RNAs

| Name | Characteristic | Sequence |
|---|---|---|
| dvssj1 labeled | Sense | UCCUUGAUAUCCGGUUCGGUA (SEQ ID |
| siRNA | | NO: 67) |
| | Anti-sense | AGGAACUAUAGGCCAAGCCAU (SEQ ID |
| | | NO: 68) |
| dvssj1 labeled | Forward | ATAATAAGTTCGATTTTTTACGAAAATG |
| frag1 dsRNA | primer | (SEQ ID NO: 69) |
| | Reverse | TACGAATACGCCGGAAGC (SEQ ID |
| | primer | NO: 70) |

### Example 2: Preparation of Cell-Penetrating Peptides

A subset of cell-penetrating peptides (CPPs) from Table 3 were prepared for use in insect cell line or whole-insect assays. A more complete list of CPPs that could be used to conjugate to an RNA molecule are provided in Table 1 and Table 2. Linear CPPs were ordered from GenScript (Piscataway, NJ) with and without fluorescent modifications. Stocks of unlabeled CPPs or CPPs conjugated to FAM dye were received lyophilized on dry ice, were reconstituted in water, and stored at −80° C. Working stocks were diluted in water as necessary for treatment preparation. The CPP-YFP fusion stocks were received stored in phosphate buffered saline (PBS), 1000 Glycerol, pH 7.4 at −80° C., and working stocks were diluted in molecular biology grade water as necessary for treatment preparation. Branched amphiphilic peptide capsules (BAPCIs) were ordered from Phoreus™ Biotechnology, Inc. (Olathe, KS), and received pre-formed from a mixture of equimolar portions of two branched CPPs (b-CPPs). BAPC is were received lyophilized on dry ice, reconstituted in water, and stored at 25° C. Concentrations and predicted characteristics of the CPPs for use in insect assays are described in Table 4.

TABLE 4
Subset of CPPs used in Example insect assays

| | | | Isoelectric | Charge at | Concentration | |
|---|---|---|---|---|---|---|
| Name | CPP sequence | Residues | point | PH 7 | (ng·μL-1) | (μM) |
| γ-zein | VRLPPPVRLPPPLVR | 19 | 12.4 | 2.91 | 1200 | 568 |
| γ-zein- | PPPL (SEQ ID NO: 3) | 19 | 12.4 | 2.91 | 4000 | 1608 |
| FAM | | | | | | |
| MPG | GALFLGFLGAAGST | 27 | 11.67 | 3.91 | 1200 | 434 |
| MPG- | MGAWSQPKSKRKV | 27 | 11.67 | 3.91 | 4000 | 1273 |
| FAM | (SEQ ID NO: 5) | 273 | 6.48 | −2.73 | 4700 | 153 |
| MPG- | | | | | | |
| YFP | | | | | | |
| Knotted- | KQINNWFINQRKRH | 16 | 12.18 | 5.01 | 1200 | 546 |
| 1 | WK (SEQ ID NO: 2) | 16 | 12.18 | 5.01 | 4000 | 1554 |
| Knotted- | | 262 | 5.49 | −7.32 | 450 | 14.9 |
| 1-FAM | | | | | | |
| Knotted- | | | | | | |
| 1-YFP | | | | | | |
| CyLoP- | CRWRWKCCKK | 10 | 10.36 | 4.71 | 1200 | 859 |
| 1 | (SEQ ID NO: 4) | 10 | 10.36 | 4.71 | 4000 | 2256 |
| CyLoP- | | | | | | |
| 1-FAM | | | | | | |
| BAPC1 | bis(FLIVI)-K-KKKK | 15 | 11.41 | 4 | 1000 | 1244 |
| (e.g., | (SEQ ID NO: 1) | 23 | | | | |
| BAP | bis(FLIVIGSII)-K- | | | | | |
| tofect) | KKKK (SEQ ID | | | | | |
| | NO: 66) | | | | | |

### Example 3: Preparation of Nanocomplex Samples

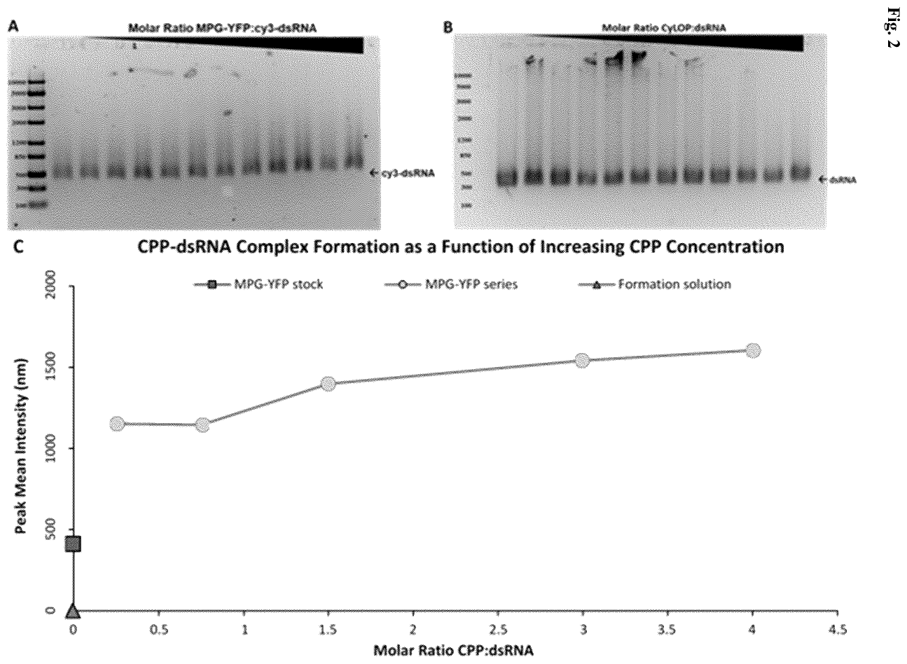

Sample preparation and analysis methods were based on those previously reported (Jafari et al. (201 4) “Serum stability and physicochemical characterization of a novel amphipathic peptide C6M 1 for siRNA delivery”, PLoS One, 9(5), e97797; and Gillet et al. (2017) “Investigating Engineered Ribonucleoprotein Particles to Improve Oral RNAi Delivery in Crop Insect Pests”, Frontiers in Physiology, 8(256), doi: 10.3389/fphys.2017.00256). Reagents are described in Examples 1 and 2. Nanocomplex samples were prepared in one of several ways, depending on type of CPP, RNA, or assay. For insect cell-based assays with CPP-FAM or CPP·mRNA, increasing amounts of CPP (from 25 μM to 100 μM, in 25 μM increments) were used alone or mixed with 20 pg mRNA in water and incubated at 25° C. for 15 minutes prior to cell treatment. Reaction volumes added to cells were 6.38 μL CPP and 6.38 μL mRNA. Final optimal CPP concentration was assessed by amount of fluorescent signal apparent within cells. For insect cell-based assays with CPP-YFP·Cy3-dsRNA or CPP·dsRNA, first a complex formation solution was prepared in molecular biology-grade water containing 90 mg·mL⁻¹ maltose, 9 mg·mL⁻¹ mannitol, 392. 16 mM CaCI₂, pH 7.0, and vacuum-filtered using Corning™ Disposable Vacuum Filter and Storage System containing a cellulose nitrate filter. Then, increasing amounts of CPP-YFP were added to Cy3-dsRNA, from a molar ratio (CPP-YFP:Cy3-dsRNA) of 1:4 through 8:1 and incubated protected from light at 25° C. for 90 minutes prior to assessment via gel shift and DLS. Reaction volumes added per cell well were comprised of 17 μL reaction solution, 1 μL CPP, 1 μL dsRNA, and 1 μL molecular biology grade water; reaction sizes for larger total volumes were increased in increments of 20 μL and component ratio was held constant to maintain an ionic strength of −1.0. Final optimal molar ratio selected for cell experiments were determined by increase of the apparent size of the Cy3-dsRNA band on a native agarose gel (FIG. 2A and FIG. 2B), and/or appearance and increase in size of the complex peak detected by a Zetasizer Ultra (Malvern Panalytical, Malvern, United Kingdom) DLS instrument (FIG. 2C). For insect cell-based assays with BAPC1Cy3-dsRNA, increasing amounts of BAPC1 were first added to 100 ng Cy3-dsRNA in water, from an N/P ratio (BAPC1:Cy3-dsRNA) of 2 and 5 through 20 in increments of 5, and incubated protected from light at 25° C. for 30 minutes. Then, 5 μL 1 μg·μL CaCl₂) was added per 45 μL volume of BAPC1:Cy3-dsRNA mix and incubated for 20 minutes prior to cell treatment. The N/P ratio is the ratio of positively-chargeable polymer amine (N=nitrogen) groups to negatively-charged nucleic acid phosphate (P=phosphate) groups. Final optimal BAPC1 concentration was assessed by amount of fluorescent signal apparent within cells.

### Example 4: Uptake of CPPs by Insect Cells

The following insect cells representing important model organism or agricultural pest phylogenetic orders were cultured to assess ability to take up CPPs: Schneider 2 (S2) from late-stage Drosophila melanogaster embryo (Diptera:Drosophilidae); IPLB-Sf21AE derivation (Sf9) from Spodoptera frugiperda ovary (fall armyworm—FAW, Lepitdoptera:Noctuidae); IPLB-DU182A (DU182A) from Diabrotica undecimpunctata embryo (southern corn rootworm—SCR, Coleoptera: Chrysomelidae); DvWL2 from Diabrotica virgifera virgifera midgut (western corn rootworm—WCR, Coleoptera: Chrysomelidae). Cells were cultured using standard reagents and protocols: S2 in Schneider's Insect medium (Sigma-Aldrich, St. Louis, MO) with 10% Gibco™ Fetal Bovine Serum (FBS), heat-inactivated (Thermo Fisher Scientific) and 0.1% Gibco™ Pluronic™ F-68 Non-ionic Surfactant (Thermo Fisher Scientific), shaking at 135 RPM and 28° C. in 125 mL non-baffled flasks with 1 inch throw (Corning, Inc., Corning, NY); SF9 in in Gibco™ Sf-900™ III SFM, 0.5% Gibco™ heat-inactivated FBS, 100 units·mL⁻¹ Gibco™ Antibiotic-Antimycotic (Thermo Fisher Scientific), shaking at 140 RPM and 27° C. in 250 mL non-baffled flasks with 1 inch throw (Corning, Inc., Corning, NY); DU182A in Gibco™ Sf-900™ SFM, 3% Gibco™ heat-inactivated FBS, 100 units·mL⁻¹ Gibco™ Antibiotic-Antimycotic (Thermo Fisher Scientific), no movement and 27° C. in 75 cm² canted-neck, vented-cap T-flasks (Corning, Inc.); DvWL2 in EX-CELL® 420 Serum-Free Medium for Insect Cells (Sigma-Aldrich) containing 9% Gibco™ heat-inactivated FBS, no movement at 28° C. in 75 cm² canted-neck, vented-cap T-flasks (Corning, Inc.). For treatment with CPPs, all cell types were pipetted gently into each well of a Costar® 24-well Clear TC-treated Multiple Well Plate (Corning, Inc.), such that 1 mL of culture gave the following cell densities per well: S2 at 2×10⁶, SF9 at 5×10⁵, DU182A at 5×10⁵, DvWL2 at 3×10⁵. To prepare for treatment and/or imaging, adherent cell types (DU182A, DvWL2) were detached from flask surfaces by first removing media, washing with trypsinization solution (0.05% Gibco™ Trypsin-EDTA in 1×PBS (Corning, Inc.)), then incubated with trypsinization solution and monitored while rocking every 1-2 minutes. Detached cell monolayers were washed with media to remove trypsinization solution and gently pipetted to suspend. Adherent cell suspensions were allowed to attach to cell wells overnight prior to treatment.

Exposure conditions of insect cells to CPPs varied depending on type and availability of CPP, detectability of fluorescent signal, and specific cell considerations. Generally, plates containing cells were centrifuged to concentrate, cell media removed, and 200 μL CPP solution in cell media were applied to each well. Cells were exposed for either 4 hours (BAPC1) or ˜25 minutes (all other CPPs) protected from light at 25° C. Cells were then washed once with media and allowed to recover overnight in their optimal conditions prior to imaging. Between four and six replicates were treated for each cell type and CPP combination across one to two 24-well cell culture plates, with one replicate equal to one well. Control treatments included cells exposed to buffer rather than CPPs. Each cell type was assessed once for post-treatment and overnight recovery survival via monitoring of oxygen consumption in 24-well OxoDish® deep-well plates using an SDR SensorDish® Reader (Applikon Biotechnology, Foster City, CA). All cell types survived long enough in sufficient quantities to give accurate fluorescent results, though in some cases handling-related morphological changes and cell death is also apparent.

To image cells, approximately 50-75 μL cells were dispensed onto a Fisherbrand™ ProbeOn™ Slide (Thermo Fisher Scientific) then coverslipped with a Richard-Allan Scientific™ Cover Glass (Thermo Fisher Scientific). Each sample was prepared immediately prior to imaging. Imaging was conducted on a Leica TCS SPE confocal microscope with LAS X software. High-resolution transmission (white-light) and fluorescent images were collected in a z-stack with sequential scanning of each wavelength for each optical slice, using several different objective lenses to cover a wide or narrow field—such as 10× or ACS APO 40×/1.15 Oil, respectively. Wavelengths used for fluorescent data collection included: FAM excitation at 488 nm with 30% laser power, emission at 493-677 nm; YFP excitation at 488 nm with 15% laser power, emission at 493-778 nm. Voltage was set for all wavelengths using buffer-treated negative control cells. Differences in fluorescent signal pattern between treatments were observed across all treatment replicates, and 2-4 images representative of the observed pattern were collected. A variety of CPPs was observed to enter into all four types of insect cells based on detection of fluorescent signal inside the cells and lack of fluorescent signal in negative controls. The imaging of fluorescent CPPs was internalized by insect cells. Increasing concentrations of CPPs were exposed to cells derived from 1 dipteran (S2), 1 lepidopteran (SF9), and 2 coleopteran (DU182A, DvWL2) insects. Exposure and imaging occurred as described in Example 4; the cells were able to uptake the CPP complex with the optimal results of 75 μM of each CPP-FAM, 55 μM of MPG-YFP, or 15 μM Knotted-1-YFP in each cell type, as indicated by red color either saturating the entire cell or localized in punctate dots or blobs. Red color was not visible in corresponding negative control treatments. These results indicate both that insect cells can internalize CPPs, and that the presence of certain labels or fusion with larger proteins is not prohibitive.

### Example 5: CPP-Mediated Delivery of mRNA Cargo into Insect Cells

Insect cells were cultured as described in Example 4 and used to assess ability of CPPs to deliver mRNA cargo. Formation of CPP·mRNA complexes is described in Example 3. Reagents and supplies, cell treatment conditions, recovery time, handling, preparation for imaging, and imaging were as described in Example 4, except wavelengths used for fluorescent data collection exclusively included: mCherry excitation at 532 with 30% laser power, emission 551-800 nm. Control treatments included cells exposed to only buffer, and to the same amount of either unlabeled CPPs or mRNA alone as was used to form the CPP·mRNA complex. Translation of fluorescent protein from CPP-delivered mRNA was observed with the tested CPPs and insect cell lines, based on detection of fluorescent signal inside the cells and lack of fluorescent signal in buffer-, CPP-, or mRNA-only treated cells. The detection of fluorescent protein translated from CPP-delivered mRNA within insect cells was observed via microscopy. Insect cells (SF9, DU182A, DvWL2) were exposed to CPP·mRNA complexes. Exposure and imaging occurred as described in Examples 4 and 5; the results obtained using complexes formed with 75 μM of three different unlabeled CPPs (MPG, Knotted-1, CyLoP) and 10 μg mCherry mRNA were confirmed visually through the microscope. Translation of mRNA into a functional fluorescent protein occurred overnight in each cell type, indicated by punctate magenta dots. Magenta dots are not visible in corresponding negative control treatments—representative negative controls images are shown.

These results indicate that not only can CPPs carry nucleic acid cargo such as mRNA into insect cells, but that the cargo can be functionally active. Cellular internalization of CPPs is thought to occur via endocytosis; if true in insects, to see translation of correctly-folded protein would likely require release of the mRNA cargo from endocytic vesicles into the cytoplasm.

### Example 6: CPP-Mediated Enhancement of dsRNA Uptake into Insect Cells

Insect cells were cultured as described in Example 4 and used to assess ability of CPPs to increase the amount of dsRNA cargo delivered. Formation of CPP·Cy3-dsRNA complexes is described in Example 3. Reagents and supplies, cell treatment conditions, recovery time, handling, preparation for imaging, and imaging were generally as described in Example 4 with two exceptions. The amount of time cells were exposed to Cy3-dsRNA alone or CPP·Cy3-dsRNA complexes was varied, and wavelengths used for fluorescent data collection included: YFP excitation at 488 nm with 15% laser power, emission at 493-778 nm; Cy3 excitation at 532 nm with 25% laser power, emission 537-758 nm. Control treatments included cells exposed only to the following individual components: buffer, Cy3-dsRNA, Cy3-siRNA, or CPP. The same amount of Cy3-dsRNA or CPP or Cy3-siRNA dsRNA-equivalent—was used in these control treatments as was used to form CPP·dsRNA complexes. A higher amount of Cy3 was observed in cells treated with CPP·dsRNA complex versus cells treated with dsRNA alone, based on amount of fluorescent signal inside the cells. Low or no fluorescent signal of YFP or Cy3 was observed in buffer-, Cy3-dsRNA-, Cy3-siRNA-, or CPP-only treated cells. The visualization of increased fluorescent dsRNA internalization by insect cells in the presence of CPP was completed using microscopy. Insect cells (S2) were exposed to CPP·Cy3-RNA complexes. Exposure and imaging occurred as described in Examples 4 and 6; results obtained using complexes formed under conditions optimal for the specific CPPs and Cy3-dsRNA tested were confirmed via microscopy. Two images per treatment were shown viewed via microscopy. Complexation reactions between 8.8×10⁻¹ nM MPG-YFP (237 ng) and 2.2×10⁻¹ nM Cy3-dsRNA (595.8 ng) were assembled at a 4:1 molar ratio as described in Example 3, and were diluted 10-fold with complexation solution immediately prior to cell application. The dsRNA- and siRNA-only control treatments were similarly diluted prior to use. Complexation reactions between 17.6 μg BAPC1 and 100 ng Cy3-dsRNA were assembled at an N/P ratio of 15 as described in Example 3. In both these series of microscopy studies in increased internalization of labeled dsRNA can be seen in cells treated with CPP·Cy3-dsRNA when compared to those treated with Cy3-dsRNA alone at identical doses of Cy3-dsRNA for the same amount of time, as indicated by amount of red punctate dots present was viewed via microscoy. Red dots are not visible in corresponding single-component treatments. The difference in amount of fluorescent signal detectable in cells treated with CPP·Cy3-dsRNA and Cy3-dsRNA alone is observable but much less pronounced at higher concentrations or longer incubation periods (data not shown).

Insect cells can take up naked dsRNA without the presence of transfection agents or other assisting molecules. These results indicate that CPPs can mediate an increased amount of nucleic acid cargo entering insect cells at a given concentration or within a certain amount of time when compared with naked nucleic acid.

### Example 7: Oral Toxicity Screening of CPPs in Whole WCR

Either unlabeled CPPs or positive control peptide were incorporated into artificial insect diet for toxicity screening against western corn rootworm (WCR) larvae as previously described, with some modifications (Zhao, J.-Z. et al. (2016) “mCry3A-selected western corn rootworm (Coleoptera: Chrysomelidae) colony exhibits high resistance and has reduced binding of mCry3A to midgut tissue” J. Econ. Entomol. 109, 1369-1377, doi: 10.1093/jee/tow049). Briefly, CPPs were incorporated into standard WCR artificial diet in 96-well microtiter plate format. A 25 μL aliquot of dosing solution was combined with 75 μL molten low-melt WCR diet and shaken on an orbital shaker to mix. Final CPP or positive control peptide concentrations in diet were 0 through 200 ppm, as shown in Table 5. Once diet solidified, preconditioned first instar WCR (freshly-hatched insects placed on neutral diet for 24 hours prior to transfer to test diet) were added to the diet plates at a rate of 1 insect·well⁻¹. Plates were placed inside an incubator (Percival Scientific, Inc., Perry, IA) set to 27° C., 65% RH, and a 24-hour dark cycle. The assay was scored for mortality and stunting affects after 7 and 12 days, without diet refresh, on a 0-3 scale, where 3=mortality, 2=severe stunting, 1=stunting, 0=no affect when compared with untreated larvae. The assay was repeated three times and end-results were tabulated using all three replicates and converted to percentages (Table 5). No mortality or stunting effects were observed for any CPP at either time point at any tested dose, whereas the positive control peptide exhibited >73% affected larvae starting at 25 ppm and >75% mortality starting at 50 ppm.

### Example 8: CPP-Mediated Enhancement of dsRNA Uptake into Insect Cells

Insect cells were cultured as described in Example 4 and used to assess ability of CPPs to increase the amount of dsRNA cargo delivered. Formation of CPP:Cy3-dsRNA complexes as described in Example 3. Reagents and supplies, cell treatment conditions, recovery time, handling, preparation for imaging, and imaging were generally as described in Example 4. Wavelengths used for fluorescent data collection included: Cy3 excitation at 532 nm with 25% laser power, emission 537-758 nm. Control treatments included cells exposed only to the following individual components: buffer, Cy3-dsRNA, Cy3-siRNA, or CPP. The same amount of Cy3-dsRNA or CPP—or Cy3-siRNA dsRNA-equivalent—was used in these control treatments as was used to form CPP:dsRNA complexes. A higher amount of Cy3 was observed in cells treated with CPP:dsRNA complex versus cells treated with dsRNA alone, based on amount of fluorescent signal inside the cells. Low or no fluorescent signal was observed in buffer-, Cy3-dsRNA-, Cy3-siRNA-, or CPP-only treated cells. Insect cells can uptake naked dsRNA without the presence of transfection agents or other assisting molecules. The visualization of increased fluorescent dsRNA internalization by insect cells in the presence of CPP. Insect cells (SF9) were exposed to CPP:Cy3-RNA complexes for 4 hours. Exposure and imaging occurred as described in Examples 4 and 8; results obtained using complexes formed under conditions optimal for the specific CPP and Cy3-dsRNA tested were obtained via microscopy. Two images per treatment were obtained via the microscopy study. Complexation reactions using 5.9 μg BAPC1 and 100 ng Cy3-dsRNA were assembled at a N/P ratio of 5 as described in Example 3 immediately prior to cell application. The dsRNA- and siRNA-only control treatments were prepared similarly but without BAPC1 prior to use. Increased internalization of labeled dsRNA was seen in cells treated with CPP:Cy3-dsRNA when compared to those treated with Cy3-dsRNA alone at identical doses of Cy3-dsRNA for the same amount of time, as indicated by presence and intensity of red punctate dots as viewed via microscopy. Red dots are not visible in corresponding single-component treatments. These results indicate that CPPs can promote an increased amount of nucleic acid cargo entering insect cells at a given concentration or within a certain amount of time when compared with naked nucleic acid.

### Example 9: Effect of CPPs on Activity of dsRNA in Susceptible Coleopteran Insect Bioassay

This example illustrates assaying coleopteran insects susceptible to externally introduced dsRNA for CPP-enhanced dsRNA activity. Double-stranded RNA targeting one or more genes essential to the coleopteran lifecycle is prepared. Treatments comprised of naked dsRNA or dsRNA complexed to either fluorescently-labeled or unlabeled CPPs are prepared across a range of dsRNA doses and CPP:dsRNA ratios. The complexed CPP:dsRNA is combined with sugar to promote feeding, and dyed with food coloring. Treatments are then fed by droplets to starved first-instar coleopteran insects for variable lengths of time, after which larvae positively identified as having fed-via visible presence of food coloring within the larval body—are transferred to standard artificial diet and reared as normal until the appropriate assay endpoint, based on role of the target gene(s). Measurements of activity based on the gene target such as mortality, stunting, or reproductive effects on the susceptible coleopteran insect pest are recorded and used to determine effectiveness of CPP enhancement of dsRNA at a given dose, complex composition, and exposure time. Molecular and biochemical methods such as RT-qPCR, Northern blot, Western blot, or enzymatic activity assays are also used to confirm knockdown of transcript(s) and/or protein(s). The results indicate that an RNA complex comprising a cell-penetrating peptide and an RNA molecule inhibiting the growth of an insect.

### Example 10: Effect of CPPs on Activity of dsRNA in Resistant Coleopteran Insect Bioassay

This example illustrates assaying coleopteran insects resistant to externally introduced dsRNA for CPP-enhanced dsRNA activity. Double-stranded RNA targeting one or more genes essential to the coleopteran lifecycle is prepared. Treatments comprised of either naked dsRNA or dsRNA complexed to either fluorescently-labeled or unlabeled CPPs are prepared across a range of dsRNA doses and CPP:dsRNA ratios, combined with sugar to promote feeding, and dyed with food coloring. Treatments are then fed by droplets to starved first-instar coleopteran insects for variable lengths of time, after which larvae positively identified as having fed-via visible presence of food coloring within the larval body-are transferred to standard artificial diet and reared as normal until the appropriate assay endpoint, based on role of the target gene(s). Measurements of activity based on the gene target such as mortality, stunting, or reproductive effects on the resistant coleopteran insect pest are recorded and used to determine effectiveness of CPP enhancement of dsRNA at a given dose, complex composition, and exposure time. Molecular and biochemical methods such as RT-qPCR, Northern blot, Western blot, or enzymatic activity assays are also used to confirm knockdown of transcript(s) and/or protein(s).

### Example 11: Effect of CPPs on Activity of dsRNA in Lepidopteran Insect Bioassay

This example illustrates assaying lepidopteran insects resistant to externally introduced dsRNA for CPP-enhanced dsRNA activity. Double-stranded RNA targeting one or more genes essential to the lepidopteran lifecycle is prepared. Treatments comprised of either naked dsRNA or dsRNA complexed to either fluorescently-labeled or unlabeled CPPs are prepared across a range of dsRNA doses and CPP:dsRNA ratios, combined with sugar to promote feeding, and dyed with food coloring. Treatments are then fed by droplets to starved first-instar lepidopteran insects for variable lengths of time, after which larvae positively identified as having fed-via visible presence of food coloring within the larval body-are transferred to standard artificial diet and reared as normal until the appropriate assay endpoint, based on role of the target gene(s). Measurements of activity based on the gene target such as mortality, stunting, or reproductive effects on the resistant lepidopteran insect pest are recorded and used to determine effectiveness of CPP enhancement of dsRNA at a given dose, complex composition, and exposure time. Molecular and biochemical methods such as RT-qPCR, Northern blot, Western blot, or enzymatic activity assays are also used to confirm knockdown of transcript(s) and/or protein(s).

While the present disclosure may be susceptible to various modifications and alternative forms, specific embodiments have been described by way of example in detail herein. However, it should be understood that the present disclosure is not intended to be limited to the particular forms disclosed. Instead, the present disclosure is to cover all modifications, equivalents, and alternatives falling within the scope of the present disclosure as defined by the following appended claims and their legal equivalents.

TABLE 5
Determination of CPP toxic activity in WCR susceptible to exogenous dsRNA

| | | γ-zein | | | MPG | | | Positive control peptide | | |
|---|---|---|---|---|---|---|---|---|---|---|
| | | | | Total | | | Total | | | Total |
| | Dose | Mortality | Affected | tested | Mortality | Affected | tested | Mortality | Affected | tested |
| TMT | (ppm) | (%) | (%) | larvae | (%) | (%) | larvae | (%) | (%) | larvae |
| 1 | 200 | 0 | 0 | 31 | 0 | 0 | 31 | 100 | 100 | 16 |
| 2 | 100 | 0 | 0 | 32 | 0 | 0 | 32 | 87.5 | 100 | 16 |
| 3 | 50.0 | 0 | 0 | 32 | 0 | 0 | 32 | 75.0 | 100 | 16 |
| 4 | 25.0 | 0 | 0 | 30 | 0 | 0 | 31 | 46.7 | 73.3 | 15 |
| 5 | 12.5 | 0 | 0 | 32 | 0 | 0 | 31 | 0 | 6.25 | 16 |
| 6 | 6.25 | 0 | 0 | 32 | 0 | 0 | 30 | na | na | na |
| 7 | 3.13 | 0 | 0 | 32 | 0 | 0 | 32 | na | na | na |
| 8 | 1.56 | 0 | 0 | 32 | 0 | 0 | 32 | na | na | na |
| 9 | 0.781 | 0 | 0 | 32 | 0 | 0 | 32 | na | na | na |
| 10 | 0.391 | 0 | 0 | 32 | 0 | 0 | 32 | na | na | na |
| 11 | 0.195 | 0 | 0 | 32 | 0 | 0 | 32 | na | na | na |
| 12 | 0.000 | 0 | 0 | 32 | 0 | 0 | 32 | 0.0 | 0.0 | 16 |

| | | Knotted-1 | | | CyLoP-1 | | |
|---|---|---|---|---|---|---|---|
| | | | | Total | | | Total |
| | Dose | Mortality | Affected | tested | Mortality | Affected | tested |
| TMT | (ppm) | (%) | (%) | larvae | (%) | (%) | larvae |
| 1 | 200 | 0 | 0 | 30 | 0 | 0 | 32 |
| 2 | 100 | 0 | 0 | 30 | 0 | 0 | 32 |
| 3 | 50.0 | 0 | 0 | 32 | 0 | 0 | 32 |
| 4 | 25.0 | 0 | 0 | 32 | 0 | 0 | 32 |
| 5 | 12.5 | 0 | 0 | 32 | 0 | 0 | 32 |
| 6 | 6.25 | 0 | 0 | 32 | 0 | 0 | 32 |
| 7 | 3.13 | 0 | 0 | 32 | 0 | 0 | 32 |
| 8 | 1.56 | 0 | 0 | 31 | 0 | 0 | 32 |
| 9 | 0.781 | 0 | 0 | 32 | 0 | 0 | 32 |
| 10 | 0.391 | 0 | 0 | 32 | 0 | 0 | 32 |
| 11 | 0.195 | 0 | 0 | 32 | 0 | 0 | 32 |
| 12 | 0.000 | 0 | 0 | 30 | 0 | 0 | 32 |

What is claimed is:

1. An RNA complex comprising a cell-penetrating peptide and an RNA molecule, wherein the cell-penetrating peptide is selected from the group consisting of SEQ ID NO:1 to SEQ ID NO:66, and wherein the one or more RNA molecules are selected from the group consisting of:
a. an RNAi-mediating molecule;
b. a double-stranded RNA molecule;
c. a siRNA molecule;
d. a micro-RNA molecule; and,
e. a mRNA molecule.

2. The RNA complex of claim 1, wherein the RNA molecule is linked to the cell-penetrating peptide via a covalent bond.

3. The RNA complex of claim 1, wherein the RNA molecule is linked to the cell-penetrating peptide via a non-covalent bond.

4. The RNA complex of claim 1, wherein the RNA molecule is linked to the cell-penetrating peptide via an adapter or linker.

5. The RNA complex of claim 1, wherein the cell-penetrating peptide is linked to the N-terminus of the RNA molecule.

6. The RNA complex of claim 1, wherein the cell-penetrating peptide is linked to the C-terminus of the RNA molecule.

7. The RNA complex of claim 1, wherein the cell-penetrating peptide is linked internally via a peptide backbone or a side chain to the RNA molecule.

8. The RNA complex of claim 1, wherein the RNA molecule is linked to the cell-penetrating peptide at a molar ratio of between about 1:1 to about 1:1000.

9. The RNA complex of claim 1, wherein the cell-penetrating peptide is linked to the RNA molecule at a molar ratio of between about 1:1000 to 1:1.

10. A method of introducing a molecule of interest into an insect cell, the method comprising:
a. providing the insect cell;
b. interacting the cell-penetrating peptide with the RNA molecule to form the RNA complex of claim 1;
c. placing the insect cell and the RNA complex in contact with each other; and
d. allowing uptake of the RNA complex into the insect cell.

11. The method, according to claim 9, wherein interacting the RNA molecule and cell-penetrating peptide, comprises fusing the RNA molecule and cell-penetrating peptide.

12. The method according to claim 9, wherein the insect cell is selected from the group of Coleoptera, Diptera, Hymenoptera, Lepidoptera, Mallophaga, Homoptera, Hemiptera, Thysanoptera, Dermaptera, Isoptera, Anoplura, Siphonaptera, and Trichoptera.

13. The method, according to claim 11, wherein the insect cell is a Hemipteran insect cell.

14. The method according to claim 9, wherein the mRNA molecule comprises a coding sequence.

15. The method according to claim 13, wherein the coding sequence is translated to a protein.

16. The method according to claim 14, wherein the coding sequence encodes an agronomic trait.

17. The method according to claim 15, wherein the agronomic trait is an insecticidal resistance trait.

18. The method according to claim 15, wherein the agronomic trait comprises a transgenic trait.

19. The method according to claim 9, wherein the contacting is performed ex vivo, in vivo, or in vitro.

20.-60. (canceled)


SEQUENCE LISTING (70 sequences)

SEQ ID NO: 1 - protein, 10 aa, synthetic construct
  REGION 1..10: note = BAPC1 (e.g., BAP tofect Cell Penetrating Peptide
FLIVIKKKKK

SEQ ID NO: 2 - protein, 16 aa, synthetic construct
  REGION 1..16: note = Cell penetrating peptide knotted
KQINNWFINQRKRHWK

SEQ ID NO: 3 - protein, 19 aa, synthetic construct
  REGION 1..19: note = Cell penetrating peptide Gamma Zein
VRLPPPVRLPPPLVRPPPL

SEQ ID NO: 4 - protein, 10 aa, synthetic construct
  REGION 1..10: note = Cell Penetrating Peptide CyLoP
CRWRWKCCKK

SEQ ID NO: 5 - protein, 27 aa, synthetic construct
  REGION 1..27: note = Cell penetrating peptide MPG v2
GALFLGFLGAAGSTMGAWSQPKSKRKV

SEQ ID NO: 6 - protein, 9 aa, synthetic construct
  REGION 1..9: note = cell penetrating peptide
RKKRRQRRR

SEQ ID NO: 7 - protein, 18 aa, synthetic construct
  REGION 1..18: note = cell penetrating peptide
RKKRRQRRRRKKRRQRRR

SEQ ID NO: 8 - protein, 9 aa, synthetic construct
  REGION 1..9: note = cell penetrating peptide
AKKRRQRRR

SEQ ID NO: 9 - protein, 35 aa, synthetic construct
  REGION 1..35: note = cell penetrating peptide
LKRWGTIKKSKAINVLRGFRKEIGRMLNILNRRRR

SEQ ID NO: 10 - protein, 28 aa, synthetic construct
  REGION 1..28: note = cell penetrating peptide
KLFMALVAFLRFLTIPPTAGILKRWGTI

SEQ ID NO: 11 - protein, 9 aa, synthetic construct
  REGION 1..9: note = cell penetrating peptide
RRRRRRRRR

SEQ ID NO: 12 - protein, 9 aa, synthetic construct
  REGION 1..9: note = cell penetrating peptide
RRRRRRRRR

SEQ ID NO: 13 - protein, 12 aa, synthetic construct
  REGION 1..12: note = cell penetrating peptide
RRRRRRRRRRRR

SEQ ID NO: 14 - protein, 13 aa, synthetic construct
  REGION 1..13: note = cell penetrating peptide
GRRRRRRRRRPPQ

SEQ ID NO: 15 - protein, 9 aa, synthetic construct
  REGION 1..9: note = cell penetrating peptide
RKKRRQRRR

SEQ ID NO: 16 - protein, 18 aa, synthetic construct
  REGION 1..18: note = cell penetrating peptide
RKKRRQRRRRKKRRQRRR

SEQ ID NO: 17 - protein, 11 aa, synthetic construct
  REGION 1..11: note = cell penetrating peptide
KKLFKKILKYL

SEQ ID NO: 18 - protein, 22 aa, synthetic construct
  REGION 1..22: note = cell penetrating peptide
KKLFKKILKYLKKLFKKILKYL

SEQ ID NO: 19 - protein, 17 aa, synthetic construct
  REGION 1..17: note = cell penetrating peptide
TRQARRNRRRRWRERQR

SEQ ID NO: 20 - DNA, 18 bp, synthetic construct
  misc_feature 1..18: note = cell penetrating peptide
  source 1..18: mol_type = other DNA
khkhkhkhkhkhkhkhkh

SEQ ID NO: 21 - protein, 9 aa, synthetic construct
  REGION 1..9: note = cell penetrating peptide
KKKKKKKKK

SEQ ID NO: 22 - protein, 18 aa, synthetic construct
  REGION 1..18: note = cell penetrating peptide
KKKKKKKKKKKKKKKKKK

SEQ ID NO: 23 - protein, 16 aa, synthetic construct
  REGION 1..16: note = cell penetrating peptide
RQIKIFFQNRRMKFKK

SEQ ID NO: 24 - protein, 16 aa, synthetic construct
  REGION 1..16: note = cell penetrating peptide
RKKRRRESRKKRRRES

SEQ ID NO: 25 - protein, 8 aa, synthetic construct
  REGION 1..8: note = cell penetrating peptide
GRKRKKRT

SEQ ID NO: 26 - protein, 9 aa, synthetic construct
  REGION 1..9: note = cell penetrating peptide
RRRQRRKKR

SEQ ID NO: 27 - protein, 16 aa, synthetic construct
  REGION 1..16: note = cell penetrating peptide
GLRKRLRKFRNKIKEK

SEQ ID NO: 28 - protein, 18 aa, synthetic construct
  REGION 1..18: note = cell penetrating peptide
KALKKLLAKWLAAAKALL

SEQ ID NO: 29 - protein, 18 aa, synthetic construct
  REGION 1..18: note = cell penetrating peptide
QLALQLALQALQAALQLA

SEQ ID NO: 30 - protein, 18 aa, synthetic construct
  REGION 1..18: note = cell penetrating peptide
LKTLATALTKLAKTLTTL

SEQ ID NO: 31 - protein, 15 aa, synthetic construct
  REGION 1..15: note = cell penetrating peptide
RAWMRWYSPTTRRYG

SEQ ID NO: 32 - protein, 18 aa, synthetic construct
  REGION 1..18: note = cell penetrating peptide
LLIILRRRIRKQAHAHSK

SEQ ID NO: 33 - protein, 16 aa, synthetic construct
  REGION 1..16: note = cell penetrating peptide
RQIRIWFQNRRMRWRR

SEQ ID NO: 34 - protein, 22 aa, synthetic construct
  REGION 1..22: note = cell penetrating peptide
MVTVLFRRLRIRRACGPPRVRV

SEQ ID NO: 35 - protein, 16 aa, synthetic construct
  REGION 1..16: note = cell penetrating peptide
RQIKIWFQNRRMKWKK

SEQ ID NO: 36 - protein, 18 aa, synthetic construct
  REGION 1..18: note = cell penetrating peptide
VRLPPPVRLPPPVRLPPP

SEQ ID NO: 37 - protein, 14 aa, synthetic construct
  REGION 1..14: note = cell penetrating peptide
LLLFLLKKRKKRKY

SEQ ID NO: 38 - protein, 23 aa, synthetic construct
  REGION 1..23: note = cell penetrating peptide
SYFILRRRRKRFPYFFTDVRVAA

SEQ ID NO: 39 - protein, 17 aa, synthetic construct
  REGION 1..17: note = cell penetrating peptide
RAGLQFPVGRVHRLLRK

SEQ ID NO: 40 - protein, 18 aa, synthetic construct
  REGION 1..18: note = cell penetrating peptide
IAARIKLRSRQHIKLRHL

SEQ ID NO: 41 - protein, 23 aa, synthetic construct
  REGION 1..23: note = cell penetrating peptide
SYDDLRRRRKRFPYFFTDVRVAA

SEQ ID NO: 42 - protein, 26 aa, synthetic construct
  REGION 1..26: note = cell penetrating peptide
KKALLALALHHLAHLALHLALALKKA

SEQ ID NO: 43 - protein, 20 aa, synthetic construct
  REGION 1..20: note = cell penetrating peptide
GLFKALLKLLKSLWKLLLKA

SEQ ID NO: 44 - protein, 27 aa, synthetic construct
  REGION 1..27: note = cell penetrating peptide
GWTLNSAGYLLGKINLKALAALAKKIL

SEQ ID NO: 45 - protein, 40 aa, synthetic construct
  REGION 1..40: note = cell penetrating peptide
GLFKALLKLLKSLWKLLLKAGLFKALLKLLKSLWKLLLKA

SEQ ID NO: 46 - protein, 16 aa, synthetic construct
  REGION 1..16: note = cell penetrating peptide
RQIKIWFPNRRMKWKK

SEQ ID NO: 47 - protein, 15 aa, synthetic construct
  REGION 1..15: note = cell penetrating peptide
QIKIWFQNRRMKWKK

SEQ ID NO: 48 - protein, 13 aa, synthetic construct
  REGION 1..13: note = cell penetrating peptide
KMDCRWRWKCCKK

SEQ ID NO: 49 - protein, 12 aa, synthetic construct
  REGION 1..12: note = cell penetrating peptide
MDCRWRWKCCKK

SEQ ID NO: 50 - protein, 14 aa, synthetic construct
  REGION 1..14: note = cell penetrating peptide
KCGCRWRWKCGCKK

SEQ ID NO: 51 - protein, 10 aa, synthetic construct
  REGION 1..10: note = cell penetrating peptide
CRWRWKCCKK

SEQ ID NO: 52 - protein, 22 aa, synthetic construct
  REGION 1..22: note = cell penetrating peptide
TKRRITPKDVIDVRSVTTEINT

SEQ ID NO: 53 - protein, 26 aa, synthetic construct
  REGION 1..26: note = cell penetrating peptide
AEKVDPVKLNLTLSAAAEALTGLGDK

SEQ ID NO: 54 - protein, 22 aa, synthetic construct
  REGION 1..22: note = cell penetrating peptide
TKRRITPKDVIDVRSVTTKINT

SEQ ID NO: 55 - protein, 22 aa, synthetic construct
  REGION 1..22: note = cell penetrating peptide
MVRRFLVTLRIRRACGPPRVRV

SEQ ID NO: 56 - protein, 25 aa, synthetic construct
  REGION 1..25: note = cell penetrating peptide
GTKMIFVGIKKKEERADLIAYLKKA

SEQ ID NO: 57 - protein, 22 aa, synthetic construct
  REGION 1..22: note = cell penetrating peptide
KCFQWQRNMRKVRGPPVSCIKR

SEQ ID NO: 58 - protein, 13 aa, synthetic construct
  REGION 1..13: note = cell penetrating peptide
EEEAAGRKRKKRT

SEQ ID NO: 59 - protein, 15 aa, synthetic construct
  REGION 1..15: note = cell penetrating peptide
FLGKKFKKYFLQLLK

SEQ ID NO: 60 - protein, 23 aa, synthetic construct
  REGION 1..23: note = cell penetrating peptide
FLIFIRVICIVIAKLKANLMCKT

SEQ ID NO: 61 - protein, 16 aa, synthetic construct
  REGION 1..16: note = cell penetrating peptide
YIVLRRRRKRVNTKRS

SEQ ID NO: 62 - protein, 16 aa, synthetic construct
  REGION 1..16: note = cell penetrating peptide
KTVLLRKLLKLLVRKI

SEQ ID NO: 63 - protein, 16 aa, synthetic construct
  REGION 1..16: note = cell penetrating peptide
LLKKRKVVRLIKFLLK

SEQ ID NO: 64 - protein, 16 aa, synthetic construct
  REGION 1..16: note = cell penetrating peptide
KKICTRKPRFMSAWAQ

SEQ ID NO: 65 - protein, 23 aa, synthetic construct
  REGION 1..23: note = cell penetrating peptide
GIGKFLHSAKKWGKAFVGQIMNC

SEQ ID NO: 66 - protein, 14 aa, synthetic construct
  REGION 1..14: note = BAPC1 (e.g., BAP tofect Cell Penetrating Peptide
FLIVIGSIIKKKKK

SEQ ID NO: 67 - DNA, 21 bp, synthetic construct
  misc_feature 1..21: note = dvssj1 labeled siRNA sense strand
  source 1..21: mol_type = other DNA
tccttgatatccggttcggta

SEQ ID NO: 68 - DNA, 21 bp, synthetic construct
  misc_feature 1..21: note = dvssj1 labeled siRNA Anti-sense probe
  source 1..21: mol_type = other DNA
aggaactataggccaagccat

SEQ ID NO: 69 - DNA, 28 bp, synthetic construct
  misc_feature 1..28: note = dvssj1 labeled frag1 dsRNA forward primer
  source 1..28: mol_type = other DNA
ataataagttcgattttttacgaaaatg

SEQ ID NO: 70 - DNA, 18 bp, synthetic construct
  misc_feature 1..18: note = dvssj1 labeled frag1 dsRNA Reverse primer
  source 1..18: mol_type = other DNA
tacgaatacgccggaagc